(19) United States
(12) Patent Application Publication
(10) Pub. No.: US 2026/0124050 A1
(43) Pub. Date: May 7, 2026

(54) SELF-ADJUSTING SOCKET FOR LOWER LIMB PROSTHESIS

(71) Applicant: VESSL PROSTHETICS INC., London (CA)

(72) Inventors: Sydney Motz ROBINSON, London (CA); Oleksiy ZAIKA, Ilderton (CA); Sajed CELA, Lucan (CA)

(21) Appl. No.: 19/436,312

(22) Filed: Dec. 30, 2025

Related U.S. Application Data
(63) Continuation-in-part of application No. 18/897,390, filed on Sep. 26, 2024.
Application No. 18/897,390 is a continuation-in-part of application No. 18/413,606, filed on Jan. 16, 2024, now Pat. No. 12,178,725.

(51) Int. Cl. A61F 2/80; A61F 2/50; A61F 2/68

(52) U.S. Cl. CPC A61F 2/80; A61F 2/68; A61F 2002/5003; A61F 2002/503; A61F 2002/5036; A61F 2002/5073; A61F 2002/6836

(57) ABSTRACT

The present disclosure describes self-adjusting sockets for lower limb prostheses in which each step transmits motion to a resilient resistive element coupled to an actuator configured to cycle between a rest position and an actuated position. The resistive element can transmit the motion to the actuator to cycle the actuator. Each cycle of the actuator acts through a mechanical linkage to tighten the socket around the residuum, until a desired threshold tightness on the residuum is reached. After the threshold tightness is reached, the resistive element yields and absorbs the motion rather than transmitting the motion, so that the actuator ceases to cycle on each step, preventing further tightening beyond the threshold.

### CROSS-REFERENCE TO RELATED APPLICATIONS

This application is a continuation-in-part of U.S. patent application Ser. No. 18/897,390 filed Sep. 26, 2024, which is a continuation-in-part of U.S. patent application Ser. No. 18/413,606 filed on Jan. 16, 2024. This application is also related to U.S. patent application Ser. No. 19/497,677 filed Dec. 29, 2025, which is a national stage application of Patent Cooperation Treaty application PCT/CA2025/050059 filed on Jan. 15, 2025. The aforementioned applications are hereby incorporated by reference in their entireties.

### TECHNICAL FIELD

The present disclosure relates to sockets for lower limb prostheses, and more particularly to self-adjusting sockets for lower limb prostheses.

### BACKGROUND

Amputation of a limb is tragic. Fortunately, medical technology has advanced considerably, and a wide range of prosthetic limbs are now available.

A prosthetic limb typically consists of a prosthetic socket, an alignment device, one or more pylons, and an end effector. The prosthetic socket interfaces with the residual limb, or residuum, and connects it to the rest of the prosthetic limb. The alignment device typically maintains proper alignment between the socket and the rest of the prosthesis. The pylon(s) connect the socket and/or alignment device to the end effector. There may be a single pylon (e.g. for transtibial and transradial amputees) or multiple pylons (e.g. for transfemoral and transhumeral amputees) pylon(s) that connect the socket and/or alignment device to the end effector. The end effector typically replicates a foot or hand, depending on whether the prosthesis is for an upper limb (transhumeral, transradial) or lower limb (transfemoral, transtibial) amputee. In the case of a transfemoral amputee, the prosthetic limb also typically includes a knee joint.

Arguably the most important component of a prosthetic limb is the prosthetic socket. It is the sole component connecting the residuum to the rest of the prosthetic limb. Effective interface (fit) between the socket and the residuum is crucial.

A major complication in achieving an effective interface between the socket and the residuum is the fact that the size and shape of the residuum is not constant, but fluctuates over time. These fluctuations include short-term fluctuations and long-term fluctuations.

Following amputation, the edema, or swelling, decreases and muscles in the residuum may atrophy from disuse, which leads to significant changes in the residuum's volume.

These are examples of relatively long-term fluctuations, which may be accommodated straightforwardly, for example by the expedient of taking periodic measurements. In the acute phase following amputation (approximately two years post-amputation), an amputee typically requires several “check sockets” which are simple sockets that are used to check whether the fit is appropriate. Since the residuum loses significant volume from edema and muscle atrophy, amputees require a series of check sockets until their residuum volume has become sufficiently stable and does not decrease as significantly week-to-week.

Unfortunately, change in the volume of the residuum is not only an acute issue following amputation, but often persists throughout an amputee's life. Moreover, short term changes in volume are common, and the volume of the residuum can change considerably over the course of a single day or even a few hours. Factors that can affect the volume of the residuum include, but are not limited to, exercise, diet, lifestyle, and other comorbidities, as well as weather.

Since conventional prosthetic sockets are rigid and unchanging in size and shape, a change in the residuum's volume alters the socket fit, that is, the interface between the socket and residuum. Typically, an amputee will progressively lose volume over the long term, as a result of edema reduction and muscle loss, and the volume will oscillate over the short term. Activities of daily living, which include any kind of ambulation, can drive fluid out of the limb, reducing its volume.

Prosthetic socks may be used to accommodate the longer-term decreases in volume - more socks and/or thicker socks may be used as residuum volume decreases over time. However, prosthetic socks are not well suited to accommodate the shorter-term fluctuations in residuum volume, as they would require the amputee to remove their prosthetic limb, add socks on top of their residuum, and then reattach the prosthetic limb. Adding or removing prosthetic socks is extremely disruptive to an amputee's activities of daily living; they must sit down to remove their prosthetic limb and rearrange or remove articles of clothing to access their residuum and add or remove prosthetic socks appropriately. They must also bring socks with them to every destination in case the need to add or remove prosthetic socks arises. Typically, amputees must add several prosthetic socks (in some cases, over 10) to properly account for the volume they lost in their residuum.

Furthermore, even if adding or removing socks throughout the day were practical, prosthetic socks can only compensate for a finite amount of volume change, and do not accommodate changes in the shape of the residuum that may result from the volume changes. As a result, painful forces can act on a part or parts of the residuum (particularly those areas with bony protrusions).

It has been observed that daily fluctuations in residuum volume for a femoral residuum or a tibial residuum typically occur at the posterior of the residuum. One attempt to address the daily fluctuations in residuum volume is described in U.S. Pat. No. 7,655,049 to Phillips, which describes a prosthetic device having a socket with an insert having a bladder system for monitoring and compensating for volume fluctuations in a residual limb. A plurality of bladders is preferably provided, in one embodiment, substantially only on a posterior portion of the socket. The bladders may be organized into zones, with the zones being inflatable to differing pressures depending on volume fluctuations in a residual limb. Pressure sensors may be provided for each bladder or for each zone, and flow regulators may be provided to control fluid flow into or out of the bladders or zones of bladders based on readings from the pressure sensors to control volume within the insert. Alternatively, bladders can be manually inflated depending on an amputee's needs.

As can be imagined, this system requires complex sensors and electronic arrangements, which result in increased complexity and cost, or manual adjustment, which increases the inconvenience for the amputee.

### SUMMARY

Broadly speaking, present disclosure describes self-adjusting sockets for lower limb prostheses in which each step transmits motion to a resilient resistive element coupled to an actuator. The resistive element can transmit the motion to the actuator to cycle the actuator. Each cycle of the actuator acts through a mechanical linkage to tighten the socket around the residuum, until a threshold tightness on the residuum is reached. After the threshold tightness is reached, the resistive element yields and absorbs the motion rather than transmitting the motion, so that the actuator ceases to cycle on each step, preventing further tightening beyond the threshold.

In one aspect, a self-adjusting socket for a lower limb prosthesis comprises a housing, a retention mechanism, at least one actuator, and a locking mechanism. The housing comprises a residuum receptacle, and a retention mechanism is carried by the housing and configured for retaining a residuum within the residuum receptacle. The actuator(s) are carried by the housing and coupled to the retention mechanism through a respective mechanical linkage, and configured to act through the respective mechanical linkage to incrementally tighten the retention mechanism against the residuum on each cycle of the actuator(s). The locking mechanism is carried by the housing and configured to maintain tightness of the retention mechanism against the residuum after each cycle of the actuator(s). The housing is configured so that each step transmits motion to a respective resilient resistive element coupled to a respective actuator. When the tightness of the retention mechanism is below a threshold, each step transmits motion across the respective resistive element to the respective actuator to cycle the respective actuator. When the tightness of the retention mechanism has reached the threshold, on each further step the respective resistive element yields to absorb the motion, so that the respective actuator fails to cycle on each further step, inhibiting further tightening of the retention mechanism beyond the threshold.

In a preferred embodiment, the locking mechanism is a releasable locking mechanism.

A preferred embodiment of the socket further comprises a manual tightening mechanism for tightening the retention mechanism.

In an embodiment, the retention mechanism comprises at least one panel movably carried by the housing, with the panel(s) being movable inwardly and outwardly relative to the residuum receptacle and the actuator(s) is configured to act through the respective mechanical linkage to incrementally move the panel(s) inwardly to tighten the panel(s) against the residuum on each cycle of the actuator(s). In a particular embodiment, the panel(s) are a plurality of panels that are arranged circumferentially about the residuum receptacle. In a more particular embodiment, the panels are disposed in respective openings so as to be inwardly and outwardly displaceable relative to the housing. In a yet more particular embodiment, the mechanical linkage comprises at least one cable coupled to the panels, and each respective actuator is configured to incrementally increase tension in the respective cable on each cycle of the respective actuator, whereby incrementally increasing the tension on the respective cable moves the respective panels inwardly relative to the residuum receptacle.

In an embodiment, the housing carries a movable platform. The platform is reciprocally movable toward and away from the residuum receptacle between a distal position and a proximal position, and the platform is biased into the distal position. Each of the actuator(s) is carried by the housing between the residuum receptacle and the platform. The respective resistive element is trapped between the platform and the respective actuator whereby movement of the platform toward the proximal position pushes the resistive element toward the respective actuator. Reciprocal movement of the platform into the proximal position and back to the distal position cycles the respective actuator only where a resistance to compression of the respective resistive element exceeds a resistance to movement from the tension in the respective cable so that the respective resistive element transmits the movement of the platform to the respective actuator instead of yielding to the movement of the platform.

In some embodiments, each actuator comprises a rocker coupled to a respective spool, and each cycle of the rocker indexes the spool to wind the respective cable onto the spool to incrementally increase the tension in the respective cable. In particular embodiments, each rocker may comprises a respective outwardly extending actuator arm that acts as a lever to pivot the rocker, and, where the resistance to compression of the respective resistive element exceeds a resistance to movement from the tension in the respective cable, the resistive element transmits the movement of the platform into the proximal position to the actuator arm to pivot the rocker and thereby index the spool.

In some embodiments, a linearly movable cam is interposed between the resistive element and the rocker, and each actuator arm is a cam follower received in a cam slot in the cam, whereby linear movement of the cam drives rotary movement of the rocker.

In some embodiments, each actuator comprises a pinion coupled to a respective spool. Each cycle of the pinion indexes the spool to wind the respective cable onto the spool to incrementally increase the tension in the respective cable. Each resistive element is mechanically coupled to a respective rack engaging the respective pinion, and, where the resistance to compression of the respective resistive element exceeds a resistance to movement from the tension in the cable, the respective resistive element transmits the movement of the platform into the proximal position to the respective engaged with the respective pinion to rotate the respective pinion and thereby index the spool.

In some embodiments, the resistive element(s) may be at least one spring.

In another aspect, a method for securing a residuum in a socket of a lower limb prosthesis is provided. Motion from steps taken with the lower limb prosthesis is transmitted across a resilient resistive element to an actuator to cycle the actuator, where each cycle of the actuator incrementally tightens a retention mechanism against the residuum until a tightness threshold of the retention mechanism is reached. After the tightness threshold is reached, motion from further steps taken with the lower limb prosthesis is transmitted into the resistive element wherein the resistive element yields and absorbs the motion so that the actuator fails to cycle on each further step, inhibiting further tightening of the retention mechanism beyond the threshold.

In some embodiments of the method, each cycle of the actuator incrementally winds a cable around a spool to increase tension in the cable, and the cable is coupled to the retention mechanism and increasing the tension in the cable tightens the retention mechanism. In particular embodiments, increasing the tension in the cable tightens the retention mechanism by forcing a panel inwardly against the residuum.

In some embodiments, the resistive element is a spring.

In a still further aspect, a method for tightening a panel in a receptacle for a residuum is provided. The method comprises applying incremental tension across the panel to move the panel inwardly relative to the receptacle. The incremental tension is applied by transmission of movement of an end effector of a lower limb prosthesis toward the residuum through a mechanical interface to a tensioner, and the movement is transmitted to the tensioner only when a resistance of the mechanical interface exceeds a current tension applied by the tensioner.

In some embodiments, the resistance of the mechanical interface may be provided by at least one spring.

In some embodiments, the tensioner may comprise a winch.

### BRIEF DESCRIPTION OF THE DRAWINGS

These and other features will become more apparent from the following description in which reference is made to the appended drawings wherein:

FIG. 1 is a top perspective view of a first illustrative embodiment of a self-adjusting socket for a lower limb prosthesis, according to an aspect of the present disclosure;

FIG. 2 is a first side elevation view of the self-adjusting socket of FIG. 1;

FIG. 3 is a front elevation view of the self-adjusting socket of FIG. 1;

FIG. 4 is a second side elevation view of the self-adjusting socket of FIG. 1;

FIG. 5 is a rear side elevation view of the self-adjusting socket of FIG. 1;

FIG. 6 is a bottom plan view of the self-adjusting socket of FIG. 1;

FIG. 6A is a cross-sectional view taken along the line 6A-6A in FIG. 3;

FIG. 6B is a cross-sectional view taken along the line 6B-6B in FIG. 3;

FIG. 6C is a cross-sectional view taken along the line 6C-6C in FIG. 3;

FIG. 7A is a cross-sectional view taken along the line 7-7 in FIG. 3, showing a first position of the panels of the self-adjusting socket of FIG. 1, when there is slack in the cables thereof;

FIG. 7B is a cross-sectional view taken along the line 7-7 in FIG. 3, showing a second position of the panels of the self-adjusting socket of FIG. 1, when there is tension in the cables thereof;

FIG. 8A is a top plan view of the self-adjusting socket of FIG. 1, showing the first position of the panels of the self-adjusting socket when there is slack in the cables thereof;

FIG. 8B is a top plan view of the self-adjusting socket of FIG. 1, showing the second position of the panels of the self-adjusting socket when there is tension in the cables thereof;

FIG. 9 is a top perspective view of the self-adjusting socket of FIG. 1 with an actuator enclosure thereof disengaged;

FIG. 9A is a top perspective view of the actuator enclosure of FIG. 9, including portions of a winch assembly contained therein;

FIG. 10 is a transparent top perspective view of the actuator enclosure of FIG. 9, exposing the winch assembly and a movable platform;

FIG. 11 is an exploded perspective view of the actuator enclosure, winch assembly and movable platform of FIG. 10;

FIG. 12 is a top plan view of the actuator enclosure of FIG. 9, including portions of the winch assembly contained therein;

FIG. 13A is a cross-sectional view taken along the line 13-13 in FIG. 9A, showing actuator arms in a rest position with the movable platform in a distal position;

FIG. 13AE is an enlargement of FIG. 13A to show detail;

FIG. 13B is a cross-sectional view taken along the line 13-13 in FIG. 9A, showing the actuator arms in an actuated position with the movable platform in a proximal position;

FIG. 13BE is an enlargement of FIG. 13B to show detail;

FIG. 13C is a cross-sectional view taken along the line 13-13 in FIG. 9A, showing the actuator arms in the rest position with the movable platform in the proximal position;

FIG. 13CE is an enlargement of FIG. 13C to show detail;

FIG. 14 is a cross-sectional view taken along the line 14-14 in FIG. 9A;

FIGS. 15A and 15B are cross-sectional views taken along the line 15-15 in FIG. 9A;

FIG. 16 is a top perspective view of a second illustrative embodiment of a self-adjusting socket for a lower limb prosthesis, according to an aspect of the present disclosure;

FIG. 17 is an exploded perspective view of the actuator enclosure, winch assembly and movable platform of the socket of FIG. 16;

FIG. 18 is a top plan view of the actuator enclosure of FIG. 17, including portions of the winch assembly contained therein;

FIG. 19 is a cross-sectional view taken along the line 19-19 in FIG. 18;

FIG. 20A is a cross-sectional view taken along the line 20-20 in FIG. 18, showing an actuator arm in a rest position with the movable platform in a distal position;

FIG. 20B is a cross-sectional view taken along the line 20-20 in FIG. 18, showing the actuator arm in an actuated position with the movable platform in a proximal position;

FIG. 20C is a cross-sectional view taken along the line 20-20 in FIG. 18, showing the actuator arm in the rest position with the movable platform in the proximal position;

FIGS. 21A and 21B are cross-sectional views taken along the line 21-21 in FIG. 18;

FIGS. 22A and 22B are perspective views of an illustrative spool for the winch assembly of FIG. 17;

FIG. 23A is a cross-sectional view of a third illustrative embodiment of an actuator enclosure, winch assembly and movable platform according to an aspect of the present disclosure, showing actuator arms in a rest position with the movable platform in a distal position;

FIG. 23B is a cross-sectional view of the actuator enclosure, winch assembly and movable platform of FIG. 23A, showing the actuator arms in an actuated position with the movable platform in a proximal position; and

FIG. 23C is a cross-sectional view of the actuator enclosure, winch assembly and movable platform of FIG. 23A, showing the actuator arms in the rest position with the movable platform in the proximal position.

FIG. 24 is a top perspective view of a fourth illustrative embodiment of a self-adjusting socket for a lower limb prosthesis, according to an aspect of the present disclosure;

FIG. 25 is a top perspective view showing an actuator enclosure of the socket of FIG. 24;

FIG. 26 is an exploded perspective view of the actuator enclosure, winch assembly and movable platform of the socket of FIG. 24;

FIG. 27 is a cross-sectional perspective view of the actuator enclosure, winch assembly and movable platform of the socket of FIG. 24;

FIG. 28 is a cross-sectional view of the actuator enclosure, winch assembly and movable platform of the socket of FIG. 24;

FIGS. 29A and 29B are side-by-side comparison views showing, respectively, a cross-sectional view of the actuator enclosure, winch assembly and movable platform of the socket of FIG. 24 and an interior view of the winch assembly and movable platform of the socket of FIG. 24, each shown with an actuator arm in a rest position with the movable platform in a distal position;

FIGS. 30A and 30B are side-by-side comparison views showing, respectively, a cross-sectional view of the actuator enclosure, winch assembly and movable platform of the socket of FIG. 24 and an interior view of the winch assembly and movable platform of the socket of FIG. 24, each shown with the actuator arm in the rest position with the movable platform in the proximal position;

FIGS. 31A and 31B are side-by-side comparison views showing, respectively, a cross-sectional view of the actuator enclosure, winch assembly and movable platform of the socket of FIG. 24 and an interior view of the winch assembly and movable platform of the socket of FIG. 24, each shown with the actuator arm moving from the rest position toward the actuated position as the movable platform moves from the distal position toward the proximal position;

FIGS. 32A and 32B are side-by-side comparison views showing, respectively, a cross-sectional view of the actuator enclosure, winch assembly and movable platform of the socket of FIG. 24 and an interior view of the winch assembly and movable platform of the socket of FIG. 24, each shown with the actuator arm in the actuated position with the movable platform in the proximal position;

FIGS. 33A through 33C are partial perspective views showing how movement of the spool corresponds to the position of the actuator arms in cam slots as a cam moves axially;

FIG. 34A is a cross-sectional view of the actuator enclosure, winch assembly and movable platform of the socket of FIG. 24, showing a spool lock member thereof engaged with a spool thereof;

FIG. 34B is a cross-sectional view of the actuator enclosure, winch assembly and movable platform of the socket of FIG. 24, showing the spool lock member thereof disengaged from the spool thereof;

FIG. 35 is a partially transparent perspective view showing an outer shroud around the actuator enclosure and winch assembly of the socket of FIG. 24.

### DETAILED DESCRIPTION

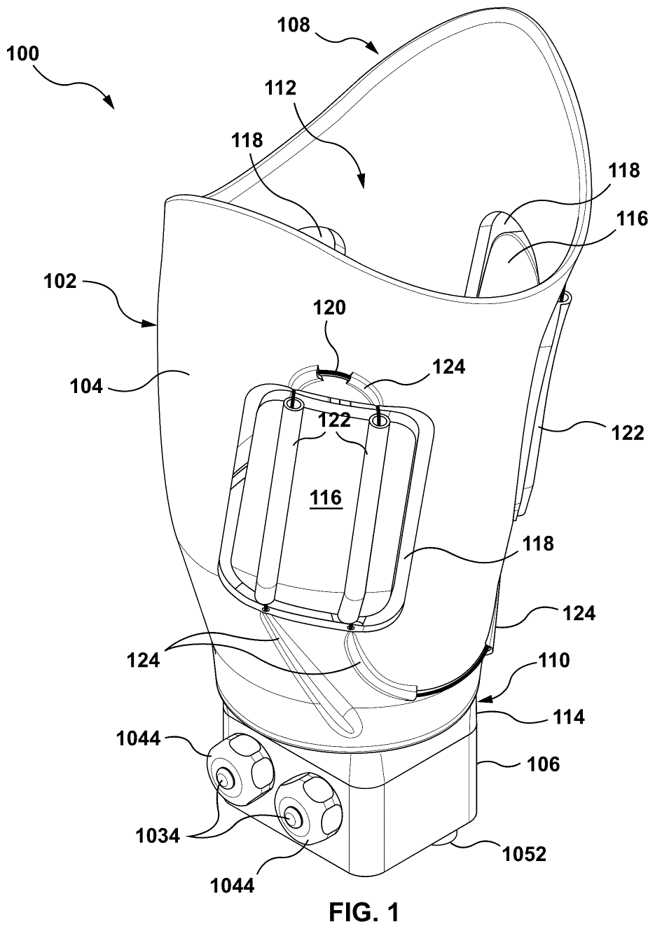

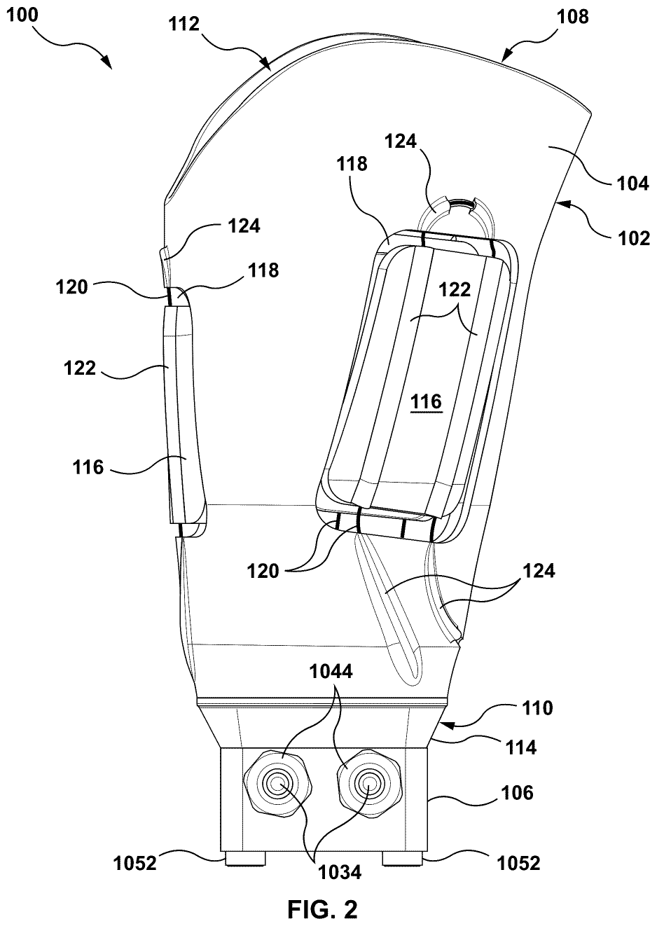

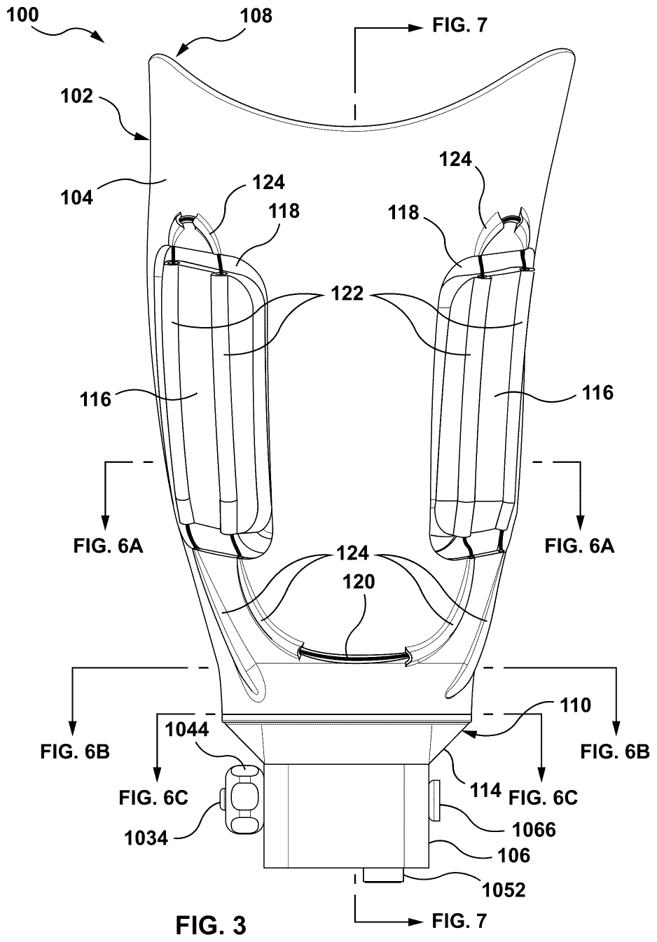

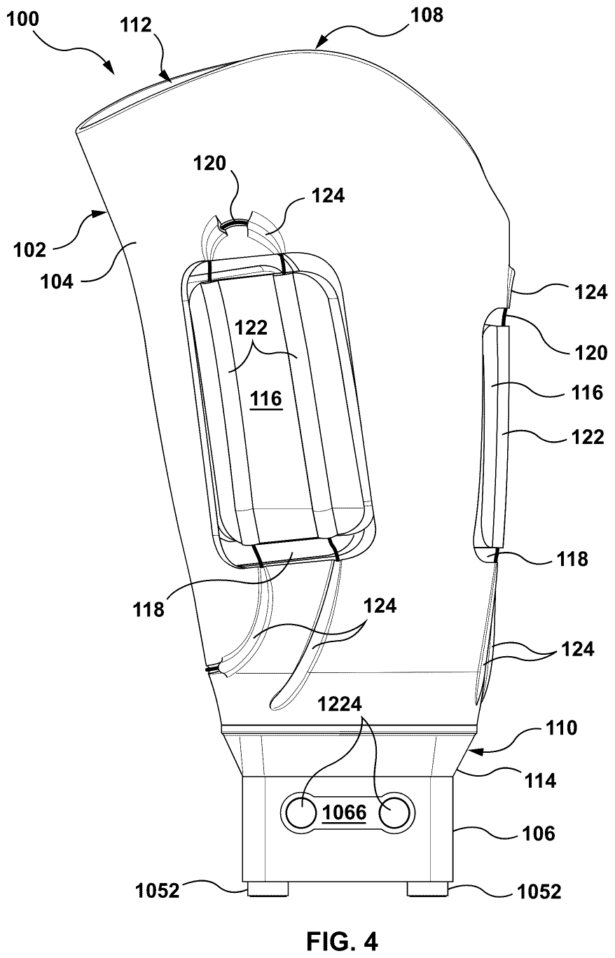

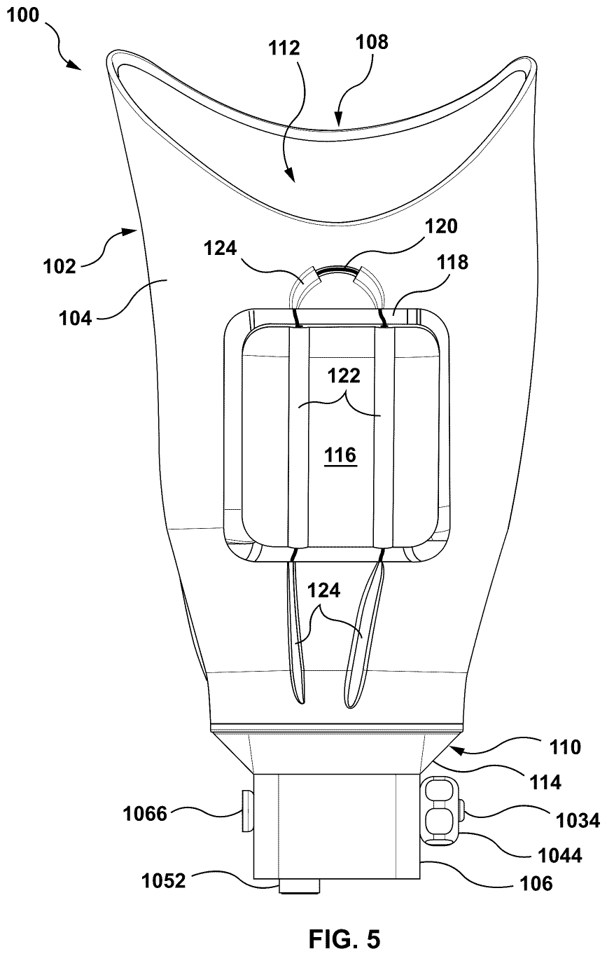

Reference is now made to FIGS. 1 to 6, which show a first non-limiting illustrative embodiment of a self-adjusting socket 100 for a lower limb prosthesis. The illustrative socket 100 is for a transtibial prosthesis, but one of ordinary skill in the art, now informed by the present disclosure, can adapt the present disclosure for use with a socket for a transfemoral amputee. Thus, sockets as described herein may be incorporated into a complete prosthetic for a transtibial or transfemoral amputee. The self-adjusting socket 100 comprises a housing 102, which in turn comprises a receptacle body 104 and an actuator enclosure 106 carried by the receptacle body 104. The receptacle body 104 is generally hollow with an open end 108 and a support end 110 opposite the open end 108, and the interior volume of the receptacle body 104 forms a residuum receptacle 112 adapted to receive the residuum of an amputee via the open end 108. Thus, the housing 102, in particular the receptacle body 104 thereof, comprises a residuum receptacle 112.

In the illustrated embodiment, a mounting block 114 is disposed at the support end 110 of the receptacle body 104 and the actuator enclosure 106 is releasably mounted to the mounting block 114. In the illustrated embodiment, the mounting block 114 is formed monolithically with the receptacle body 104, in other embodiments the mounting block may be a separate part. In still other embodiments, the actuator enclosure may be mounted directly to the support end of the receptacle body, or may be monolithically formed therewith.

The housing 102 carries a retention mechanism configured for retaining a residuum within the residuum receptacle 112. In the illustrated embodiment, the retention mechanism comprises three panels 116 movably carried by the housing 102, in particular the receptacle body 104 thereof, so that the panels 116 are movable inwardly and outwardly relative to the residuum receptacle 112. As shown, the panels 116 are arranged circumferentially about the residuum receptacle 112, and are disposed in respective openings 118 in the receptacle body 104 so as to be inwardly and outwardly displaceable relative to the receptacle body 104 of the housing 102. In other embodiments, more or fewer panels may be present, or an alternate retention mechanism may be used. The panels may include interiorly facing pads for cushioning. The panels 116 shown for the illustrative socket 100 are configured for a transtibial amputee; an alternate configuration, as will be apparent to one of ordinary skill in the art, now informed by the present disclosure, may be adapted for a transfemoral amputee.

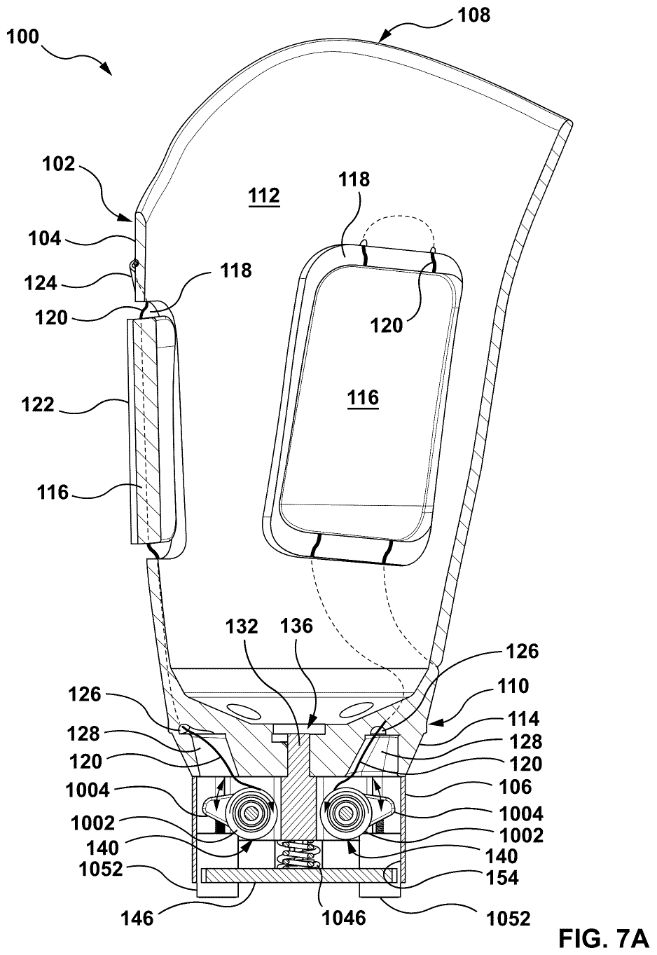

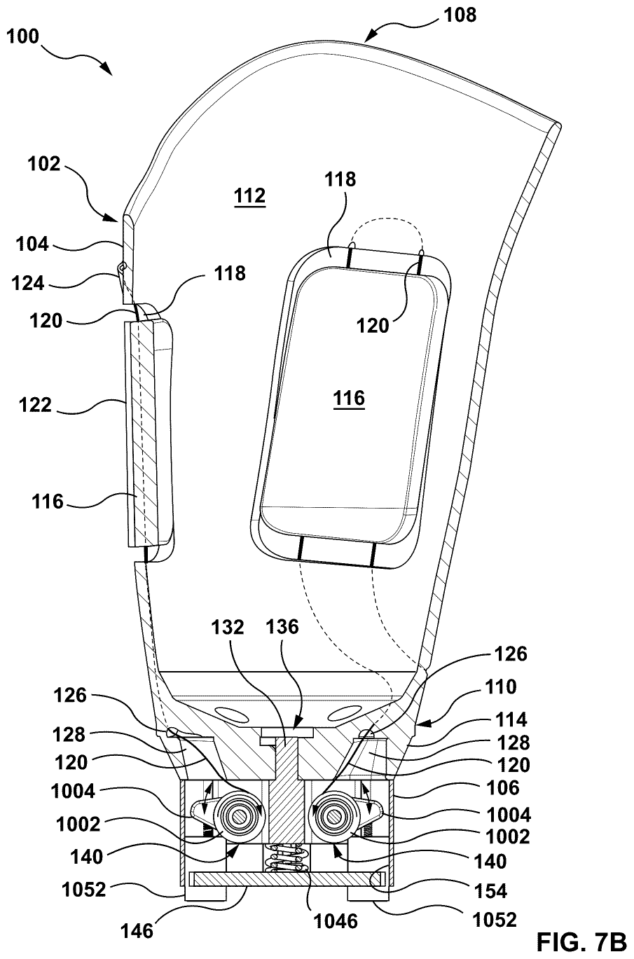

Referring now primarily to FIGS. 7A and 7B, the self-adjusting socket 100 automatically tightens the panels 116 in the residuum receptacle 112 with each step taken by the user with the lower limb prosthesis, until a desired tightness is achieved. The term “step”, as used herein, includes the act of lifting the lower limb prosthesis off of a surface and setting it down on the surface in different position with the user's weight applied, and also includes shifting the weight of the user's body off of and on to the lower limb prosthesis without lifting the lower limb prosthesis off of the surface. The self-adjusting socket 100 is configured to apply incremental tension across each panel 116 to move each panel 116 incrementally inwardly relative to the residuum receptacle 112. The incremental tension is applied by transmission of movement of the end effector of the lower limb prosthesis toward the residuum through a mechanical interface to a tensioner, with the resisted movement resulting from taking a step with (which includes shifting weight onto) the lower limb prosthesis. However, the movement from the step is transmitted to the tensioner only when the resistance of the mechanical interface exceeds a current tension applied by the tensioner, thereby preventing overtightening. Each step tightens the panels until the panels are tight enough.

In the illustrated embodiment, the tensioner applies tension to cables 120 that can be tightened or slackened. The term “cable” is used herein in its broadest sense, and includes not only braided metal rope, but also braided ropes formed from other materials, for example nylon paracord, as well as monofilament, for example fishing line, and any other suitable filar material.

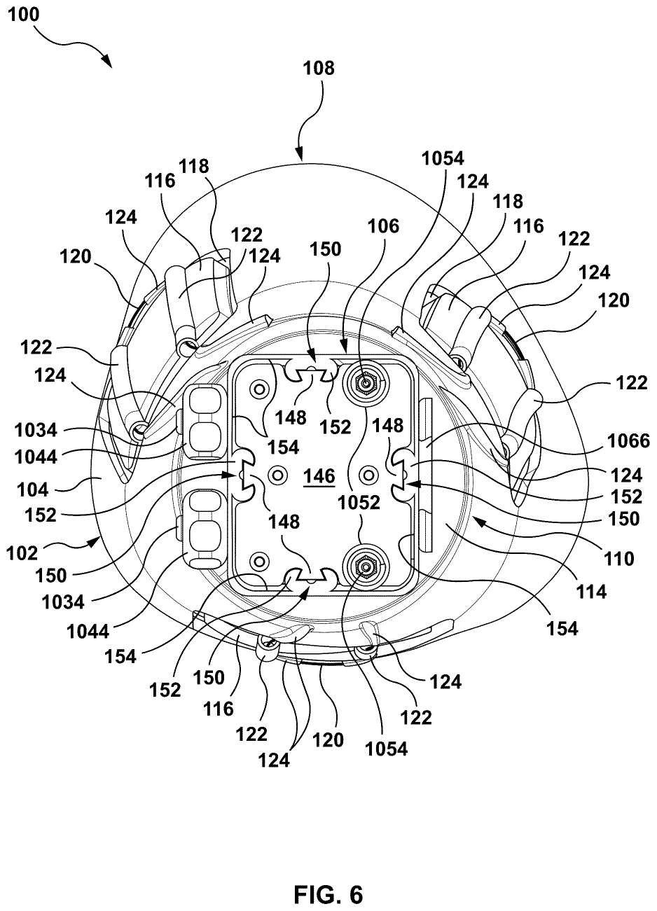

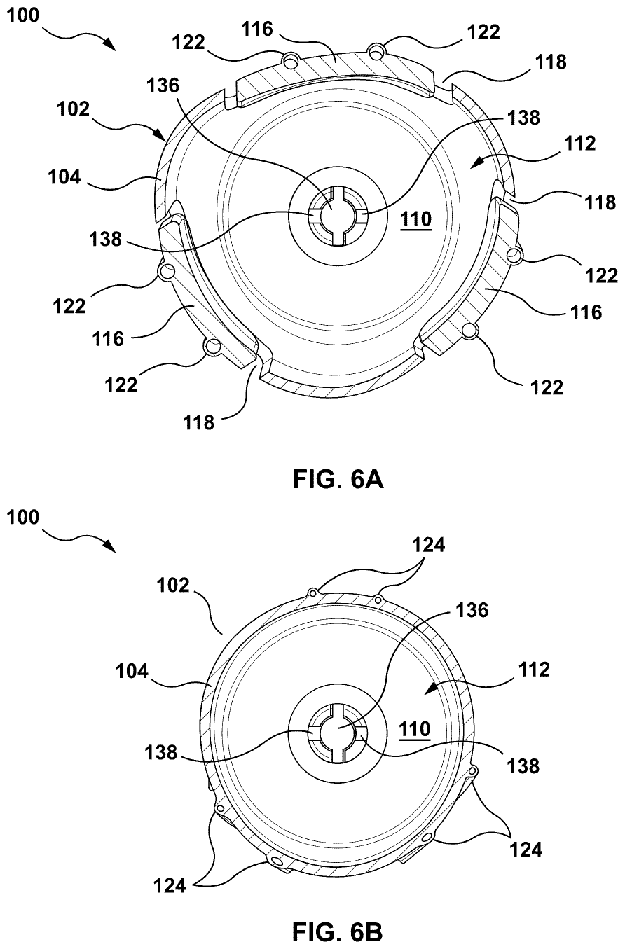

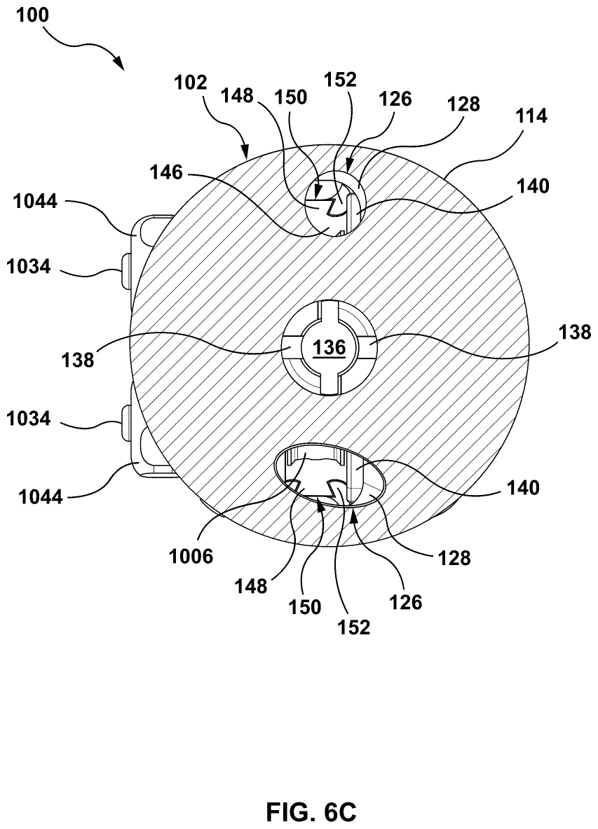

Referring now to FIGS. 6, 6A, 6B and 6C, the cables 120 run through tubular cable tunnels 122 formed in the panels 116 and through covered cable guides 124 on the outer surface of the receptacle body 104. In alternative embodiments, the cable guides may be formed within the receptacle body. The cable guides 124 guide the cables 120 through the cable tunnels 122, with one end of each cable 120 being anchored to the housing 102, and the other end of each cable 120 passing through a respective port 126 into a respective passageway 128 through the mounting block 114 at the support end 110 of the receptacle body 104. The passageways 128 lead into the actuator enclosure 106, in which the tensioner is disposed. In the illustrated embodiment, the tensioner comprises a winch assembly 130, and the ends of the cables 120 that pass through the passageways 128 are coupled to the winch assembly 130, which is adapted to apply tension to, and release tension from, the cables 120. In alternate embodiments, both ends of each cable may be coupled to the winch assembly. The cable pathways shown in the figures are merely illustrative and not intended to be limiting; a wide array of cable pathways is possible, and may vary, for example, based on the number of panels and the position of those panels.

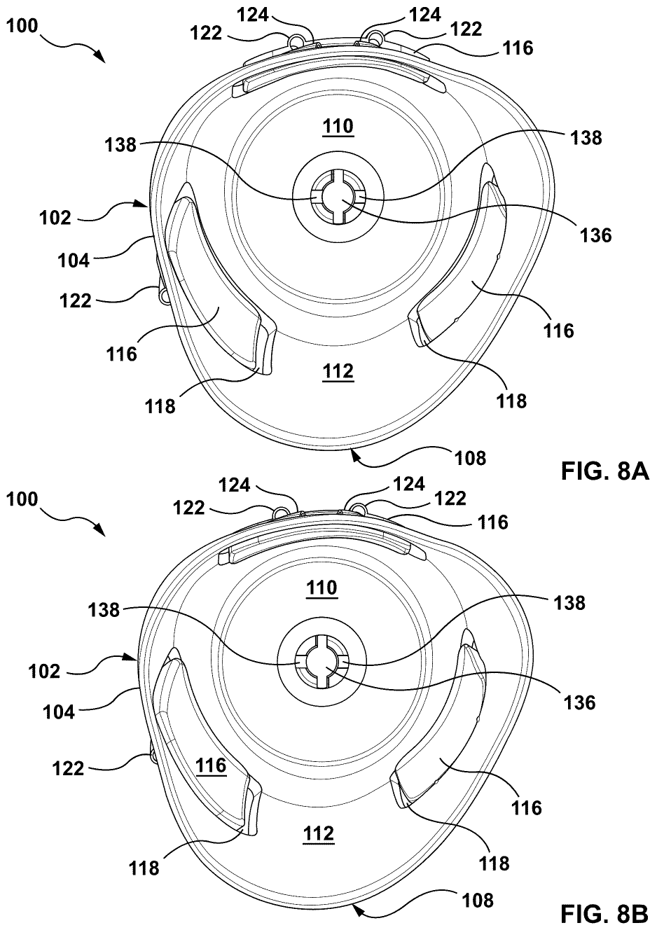

As can be seen in FIG. 7A, when there is slack in the cables 120, the panels 116 are held loosely within the respective openings 118, and will apply little or no pressure on the residuum. FIG. 8A shows the position of the panels 116 when there is slack in the cables 120, as seen from the open end 108 of the receptacle body 104. The shape (e.g. curvature) of the receptacle body 104, and the positioning of the cable tunnels 122 and cable guides 124, is such that as tension is applied and the cables 120 are tightened, the panels 116 are drawn inwardly into the openings 118, as shown in FIG. 7B, to apply pressure on the residuum. FIG. 8B shows the position of the panels 116, again from the open end 108 of the receptacle body 104, after the panels 116 have moved inwardly as a result of tightening the cables 120.

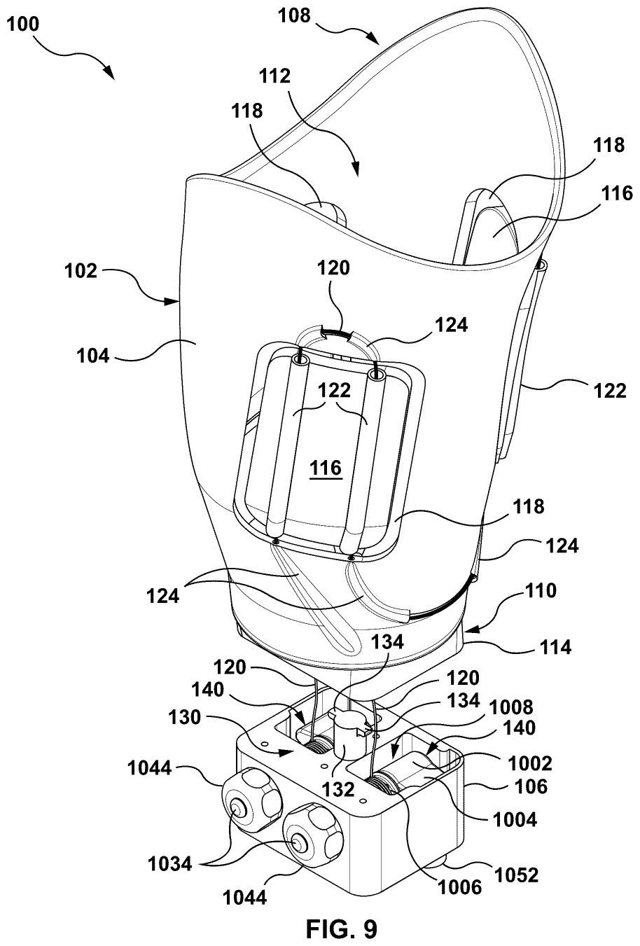

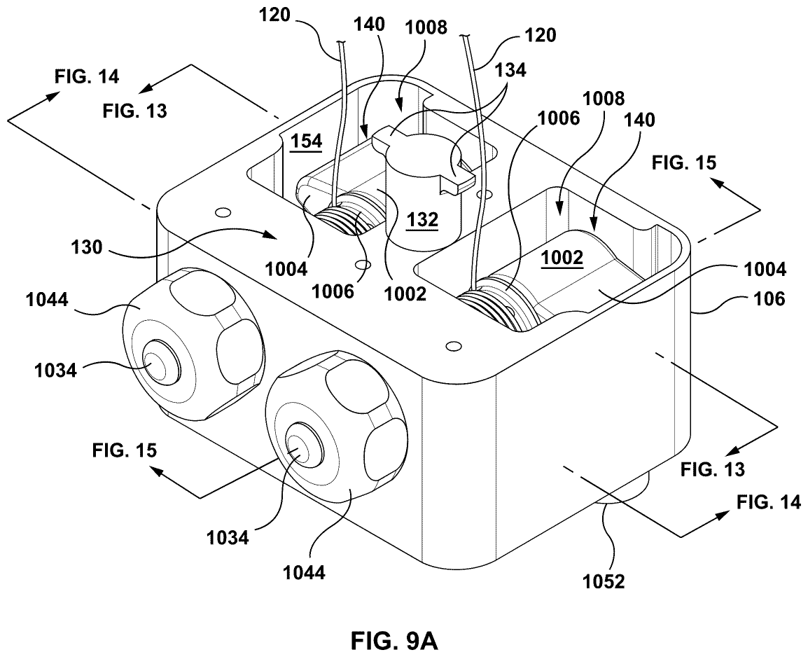

As noted above, in the illustrated embodiment the actuator enclosure 106 is releasably mounted to the mounting block 114. More particularly, as can be seen in FIGS. 9 and 9A, in a preferred embodiment a locking post 132 having diametrically opposed locking lugs 134 extending outwardly therefrom stands proud of the actuator enclosure 106. The locking post 132 may be inserted through a correspondingly shaped locking aperture 136 (see FIGS. 8A and 8B) in the support end 110 of the receptacle body 104 and then twisted to engage the locking lugs with corresponding locking recesses 138 adjacent the locking aperture 136 (see FIGS. 8A and 8B) and secure the actuator enclosure 106 to the support end 110 of the receptacle body 104. By reversing this action, the actuator enclosure 106 may be disengaged from the support end 110 of the receptacle body 104, for example for maintenance.

Continuing to refer to FIGS. 9 and 9A, and as will be described in greater detail below, in the illustrated embodiment a pair of actuators 140 are carried by the housing 102, in particular within the actuator enclosure 106; the actuators 140 are coupled to the retention mechanism, which in the illustrated embodiment comprises the panels 116, by a mechanical linkage. In the illustrated embodiment, the actuators 140 drive the winch assembly 130 carried by the actuator enclosure 106, and the mechanical linkage includes the tensioner that applies tension to the cables 120. Other types of mechanical linkage are also contemplated, for example suitable gearing. The actuators 140 are configured to act through the mechanical linkage comprising the cables 120, via the winch assembly 130, to incrementally tighten the retention mechanism comprising the panels 116 against the residuum on each cycle of the actuators 140. More particularly, each respective actuator 140 is configured to incrementally increase tension in the respective cable 120 on each cycle of the respective actuator 140, and incrementally increasing the tension in the respective cable 120 moves the respective panel(s) 116 incrementally inwardly relative to the residuum receptacle 120.

In the illustrated embodiment, the winch assembly 130 includes a releasable locking mechanism that is also carried by the housing 102, in particular within the actuator enclosure 106; the locking mechanism is configured to maintain the tightness of the retention mechanism comprising the panels 116 against the residuum after each cycle of the actuator. An illustrative locking mechanism is described further below.

Each time a user takes a step with the lower limb prosthesis, that step transmits motion to a respective resilient resistive element coupled to a respective one of the actuators 140. In the illustrated embodiment, the motion is transmitted by a movable platform 146 adapted to be coupled to an end effector and which is reciprocally movable toward and away from the residuum receptacle between a proximal position and a distal position. As used herein, the terms “proximal” and “distal” refer to relative proximity to the residuum receptacle, or to the end of the actuator enclosure that will be coupled to the residuum receptacle. The resistance of the resistive elements is calibrated to a desired tightness of the retention mechanism comprising the panels 116; preferably the resistive elements are configured so that the resistance is adjustable and one such embodiment is described below. When the tightness of the retention mechanism comprising the panels 116 is below a desired threshold, each step transmits motion across the resistive elements to the actuators 140 to cycle the actuators 140. However, when the tightness of the retention mechanism comprising the panels 116 has reached the threshold, on each further step the resistive elements yield to absorb the motion, rather than transmitting the motion to the actuators 140. When the resistive elements yield instead of transmitting motion, the actuators 140 will fail to cycle on each further step, thereby inhibiting further tightening of the retention mechanism beyond the threshold.

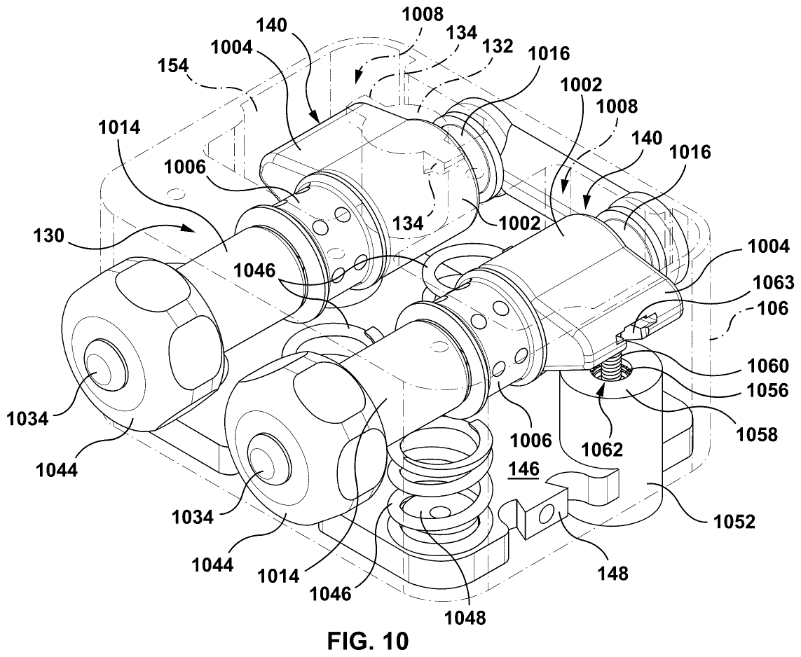

Reference is now made to FIGS. 10 to 12, which show, respectively, an assembled and an exploded view of the winch assembly 130 carried by the actuator enclosure 106, along with the movable platform 146. The actuator enclosure 106 functions as a winch body that carries the components of the winch assembly 130.

In the illustrated embodiment, the winch assembly comprises two actuators 140, with each actuator 140 comprising a rocker having a tubular cylindrical actuator body 1002 and an outwardly extending actuator arm 1004 that acts as a lever to pivot the actuator 140 so that the actuator 140 can rock back and forth about an axis extending through the actuator body 1002. The rocker is merely one non-limiting illustrative embodiment of an actuator, and other types of actuators are also contemplated.

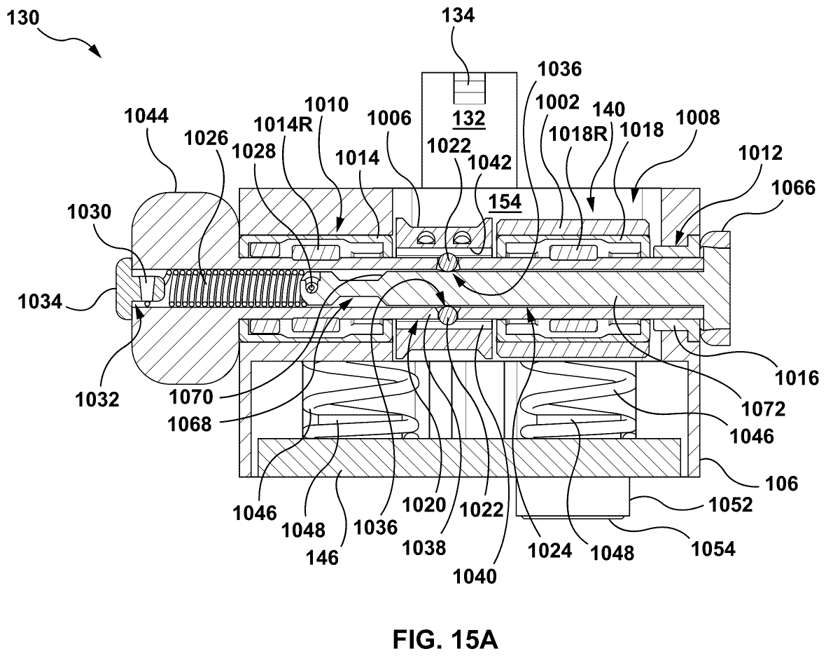

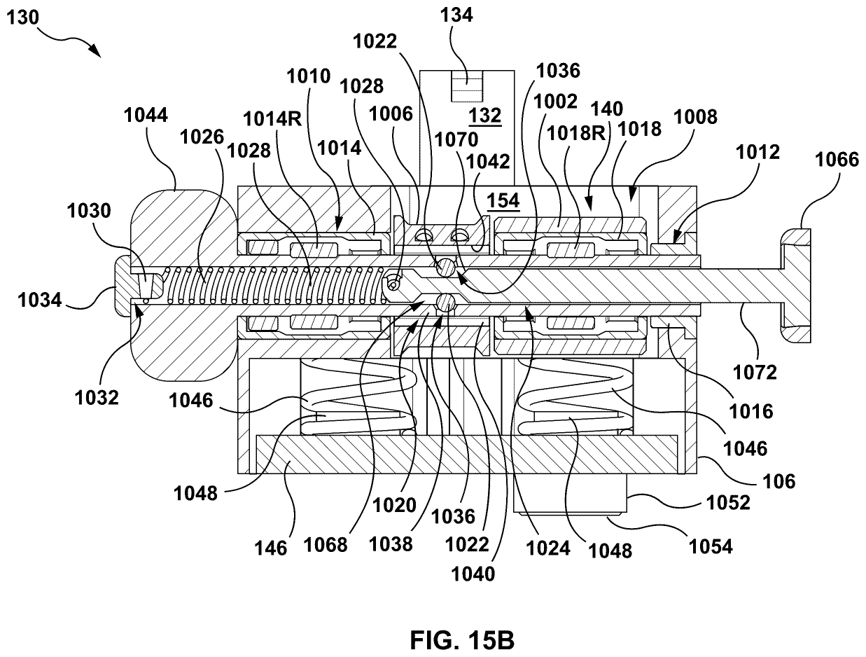

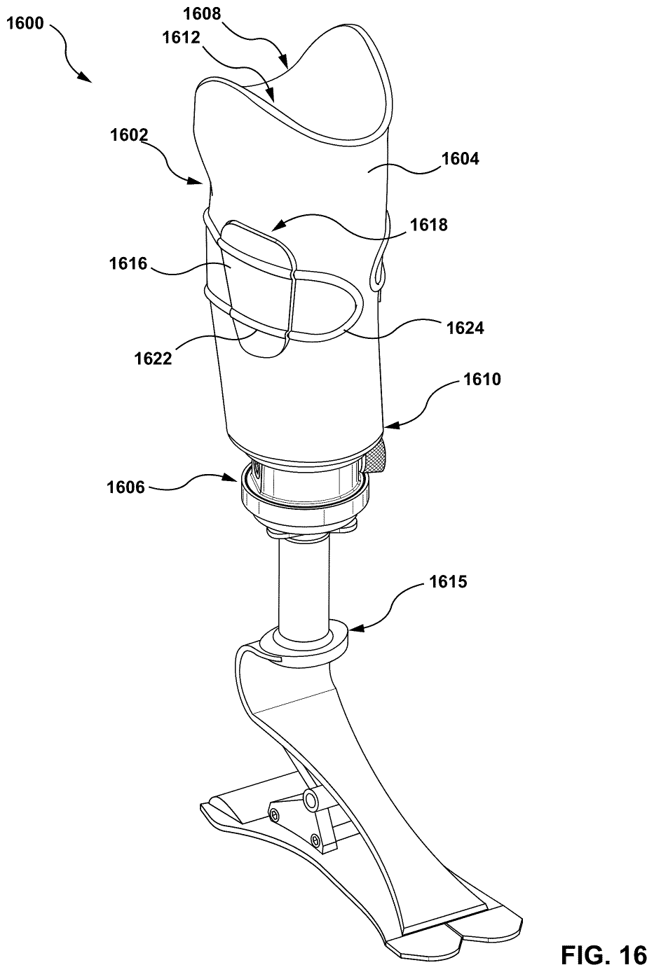

Each of the actuators 140 is coupled to a respective spool 1006 onto which the cables 120 may be wound and from which the cables 120 may be unwound to respectively tighten and loosen the retention mechanism comprising the panels 116. The actuators 140 and the spools 1006 are disposed in respective winch cavities 1008 formed within the actuator enclosure 106. A bushing aperture 1010 is disposed at one end of each winch cavity 1008 and a bearing aperture 1012 is disposed at the opposite end of each winch cavity 1008, in registration with one another. A respective winch needle bearing 1014 with winch needle bearing rollers 1014R (FIGS. 15 and 16) is friction fit into each bearing aperture 1012, and a respective winch bushing 1016 is fitted into each bushing aperture 1010, so as to form two sets wherein the respective winch needle bearing 1014 and winch bushing 1016 are arranged coaxially with one another. The winch needle bearings 1014 are one-way needle bearings. Optionally, the winch bushings may be replaced with bearings.

In the illustrated embodiment, each actuator 140 is coupled to its respective spool 1006 by way of an actuator needle bearing 1018, a hollow main winch shaft 1020, locking ball bearings 1022, and a release shaft 1024 disposed concentrically within the lumen of the hollow main winch shaft 1020. The actuator needle bearings 1018 are one-way needle bearings including actuator needle bearing rollers 1018R (FIGS. 15A and 15B), and are friction fit into the opening of the tubular cylindrical actuator body 1002. The main winch shafts 1020 each pass through a respective winch bushing 1016, respective actuator needle bearing 1018, and respective winch needle bearing 1014. The actuator needle bearings 1018 and the winch needle bearings 1014 are arranged so that the actuator needle bearing 1018 and the winch needle bearing 1014 on each main winch shaft 1020 have a common permitted direction of rotation; the permitted direction of rotation for each main winch shaft 1020 is opposite to that of the other main winch shaft 2020.

Each release shaft 1024 is axially movable within its respective main winch shaft 1020, and is coupled to the main winch shaft 1020 by a resilient member, in this case a helical extension spring which serves as a release spring 1026. More particularly, each release shaft 1024 terminates, at an end closest to the winch needle bearing 1014, with an eye 1028 adapted to receive a first hooked end of the respective release spring 1026. The other hooked end of the respective release spring 1026 is received in the eye 1030 of a spring retainer 1032. The eye 1030 of each spring retainer 1032 is disposed within the respective main winch shaft 1020, with the spring retainer 1032 being retained against the respective main winch shaft 1020 by an end cap 1034 of the spring retainer 1032. The locking ball bearings 1022 are received within respective locating apertures 1036 extending through the annular wall 1038 of the respective main winch shaft 1020. The locking ball bearings 1022 are forced outwardly by the release shaft 1024 and received within axially extending locking channels 1040 formed in the inner surface 1042 of the respective spool 1006. Because of the interengagement of the locking ball bearings 1022 with the locking channels 1040, rotation of the main winch shaft 1020 will result in rotation of the respective spool 1006.

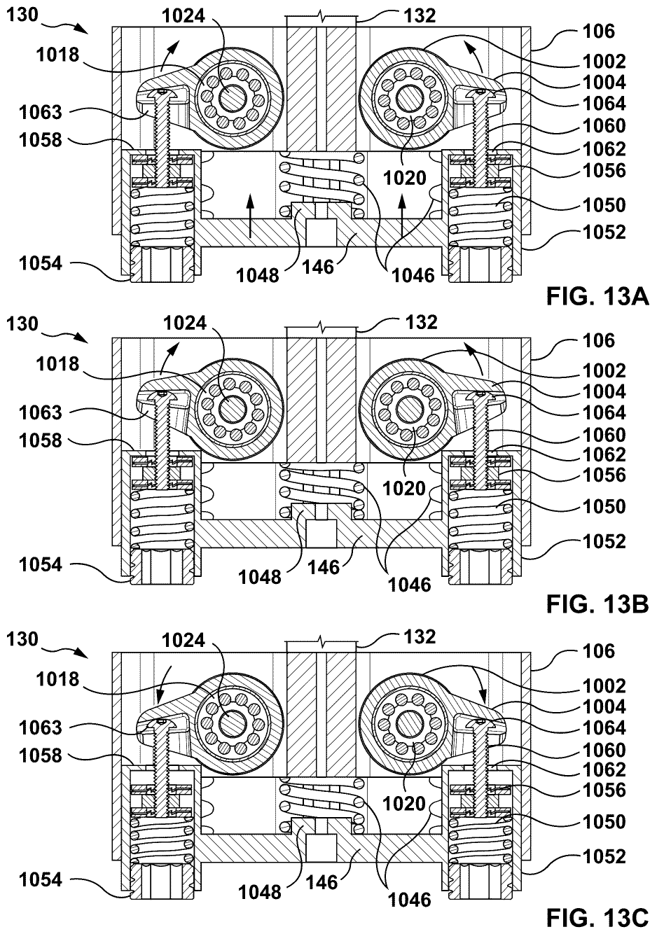

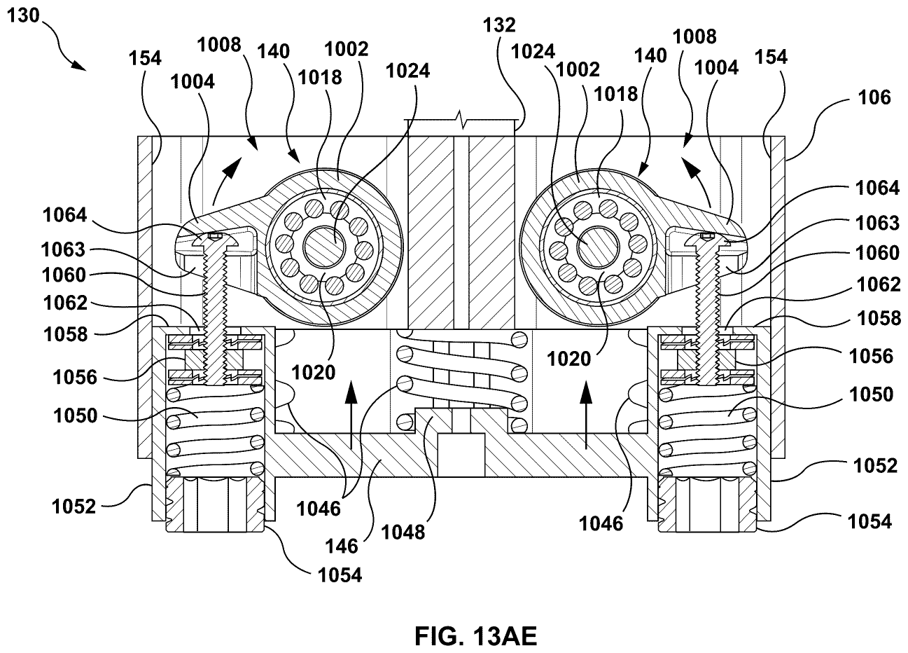

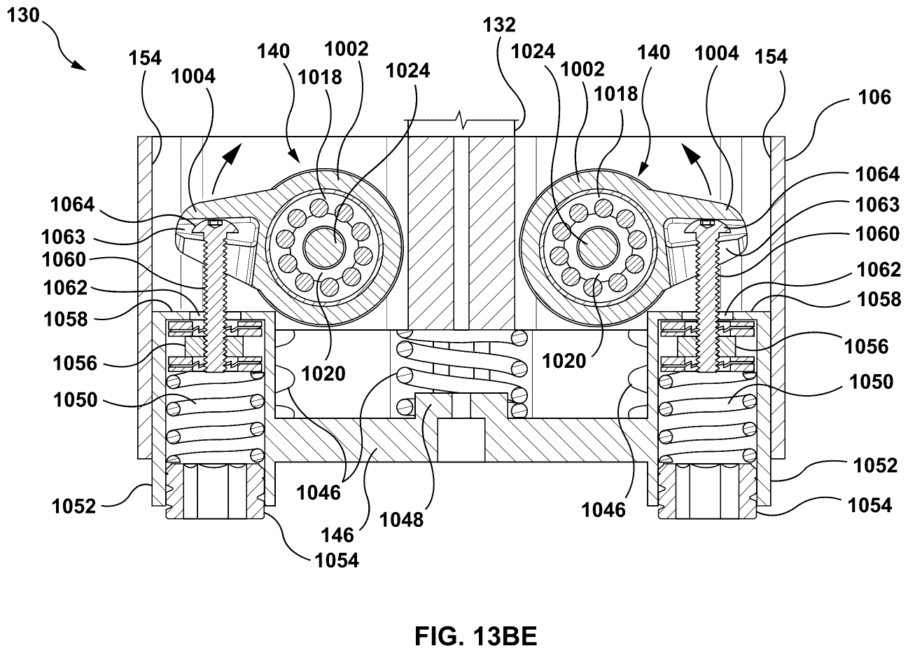

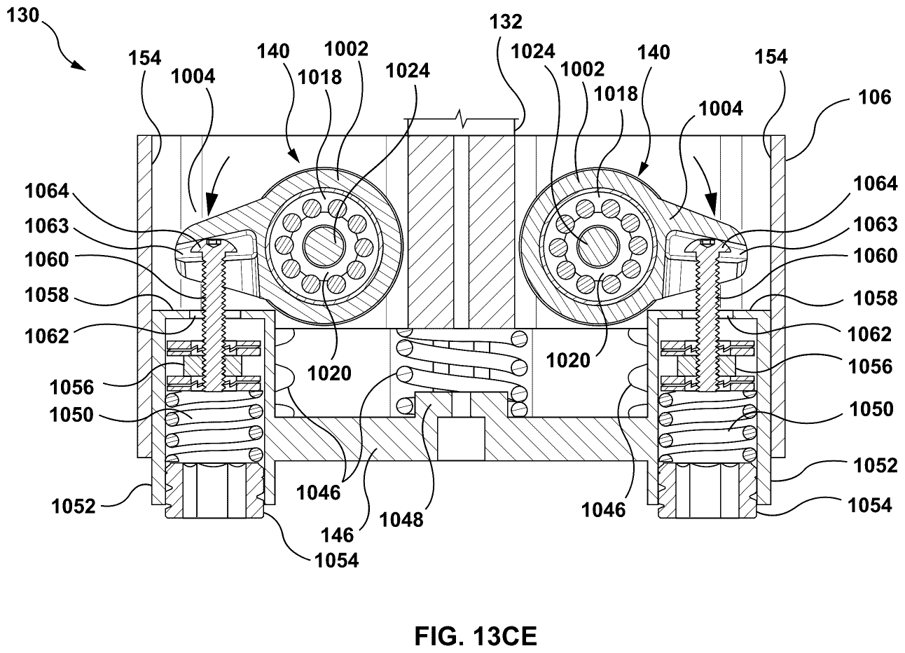

FIGS. 13A to 13C and 13AE to 13CE show various configurations of the actuator arms 140 and the movable platform 146. FIGS. 13A to 13C are positioned on the same page to facilitate comparison, and FIG. 13AE to 13CE are respective enlargements of FIGS. 13A to 13C, each on a separate page to show more detail. In the description that follows, the terms “rest position” and “actuated position” are applied to the actuator arms 1004 since the relative positions of the actuator arms 1004 are clearly visible in the drawings. It will be understood that when the actuator arm 1004 is in the rest position the actuator 140 of which the actuator arm 1004 forms a part will likewise be in the rest position, and conversely when the actuator arm 1004 is in the actuated position the actuator 140 of which the actuator arm 1004 forms a part will likewise be in the actuated position

Reference is now made to FIGS. 13A and 13AE and to FIGS. 13B and 13BE, movement of the actuator arm 1004 from a rest position (see FIGS. 13A and 13AE) to an actuated position (see FIGS. 13B and 13BE) will rotate (“rock”) the actuator body 1002 (clockwise on the left side of FIGS. 13A, 13AE, 13B and 13BE, anticlockwise on the right side of FIGS. 13A, 13AE, 13B and 13BE). Rotation of the actuator body 1002 is in the same rotational direction as the permitted direction of rotation of the respective actuator needle bearing 1018. Because the respective actuator needle bearing 1018 is friction fit within the actuator body 1002, the actuator needle bearing 1018 rotates along with the actuator body 1002. Since the actuator needle bearing 1018 is rotating in the same rotational direction that the main winch shaft 1020 is permitted to rotate within the actuator needle bearing 1018, this is equivalent to attempted rotation of the main winch shaft 1020 opposite to the permitted direction of rotation within the actuator needle bearing 1018. As a result, the actuator needle bearing 1018 binds on the main winch shaft 1020 so that rotation of the actuator body 1002 drives rotation of the main winch shaft 1020 (clockwise on the left side of FIGS. 13A, 13AE, 13B and 13BE, anticlockwise on the right side of FIGS. 13A, 13AE, 13B and 13BE). Since the actuator needle bearing 1018 and the winch needle bearing 1014 on each main winch shaft 1020 have a common permitted direction of rotation, the main winch shaft 1020 can rotate within the winch needle bearing 1014 until the actuator arm 1004 reaches the actuated position (see FIGS. 13B and 13BE). The permitted direction of rotation is in a winding direction of the respective spool 1006, so the movement of the actuator arm 1004 from the rest position (see FIGS. 13A and 13AE) to the actuated position (see FIGS. 13B and 13BE) will incrementally rotate the respective main winch shaft 1020 and the respective spool 1006 coupled thereto in a winding direction of the respective spool 1006. This incrementally winds the respective cable 120 further onto the respective spool 1006 and incrementally increases the tension in the respective cable 120. Rotation in the unwinding direction of the respective spool 1006 is resisted by the respective winch needle bearing 1014 so that tension on the respective cable 120 is maintained.

From the actuated position shown in FIGS. 13B and 13BE, the actuator arm 1004 can reciprocate back to the rest position shown in FIGS. 13A and 13AE, thereby rotating the actuator body 1002 back in the opposite rotational direction (anticlockwise on the left side of FIGS. 13A, 13AE, 13B and 13BE, clockwise on the right side of FIGS. 13A, 13AE, 13B and 13BE). This rotation of the actuator body 1002 and the respective actuator needle bearing 1018 therein is in the opposite rotational direction to the permitted direction of rotation of the respective actuator needle bearing 1018, which is equivalent to rotation of the main winch shaft 1020 in the permitted direction of rotation within the actuator needle bearing 1018. As a result, the actuator needle bearing 1018 and the actuator body 1002 can slip over the main winch shaft 1020 until the actuator arm 1004 returns to the rest position shown in FIGS. 13A and 13AE. At the same time, because rotation of the actuator body 1002 and the respective actuator needle bearing 1018 therein is in the opposite rotational direction to the common permitted direction of rotation of the respective winch needle bearing 1014, the winch needle bearing 1014 will inhibit the main winch shaft 1020 from rotating back with the respective actuator body 1002 and the respective actuator needle bearing 1018. Thus, each cycle of the actuator 140 from the rest position (FIGS. 13A and 13AE) to the actuated position (FIGS. 13B and 13BE) and back to the rest position (FIGS. 13A and 13AE) will incrementally rotate (index) the respective main winch shaft 1020. Since rotation of the main winch shaft 1020 will result in rotation of the respective spool 1006 by way of interengagement of the locking ball bearings 1022 with the locking channels 1040, each cycle of the actuator 140 from the rest position (FIGS. 13A and 13AE) to the actuated position (FIGS. 13B and 13BE) and back to the rest position (FIGS. 13A and 13AE) will incrementally rotate (index) the spool 1006. Thus, each cycle of the actuator 140 indexes the spool 1006 to wind the respective cable 120 onto the spool 1006 to incrementally increase the tension in the respective cable 120.

Reference is again made to FIGS. 10 to 12. In the illustrated embodiment, each main winch shaft 1020 terminates with a respective manual tensioning knob 1044 at the end of the main winch shaft 1020 that receives the spring retainer 1032. Preferably the manual tensioning knob 1044 is monolithically formed as part of the main winch shaft 1020; in other embodiments it may be a separate part affixed to the main winch shaft. By rotating the manual tensioning knob 1044 in the permitted direction of rotation, the main winch shaft 1020 and hence the spool 1006 may be rotated independently of the actuator 140 to manually apply tension to the cables 120. Thus, the manual tensioning knobs 1044 provide a manual tightening mechanism for tightening the retention mechanism.

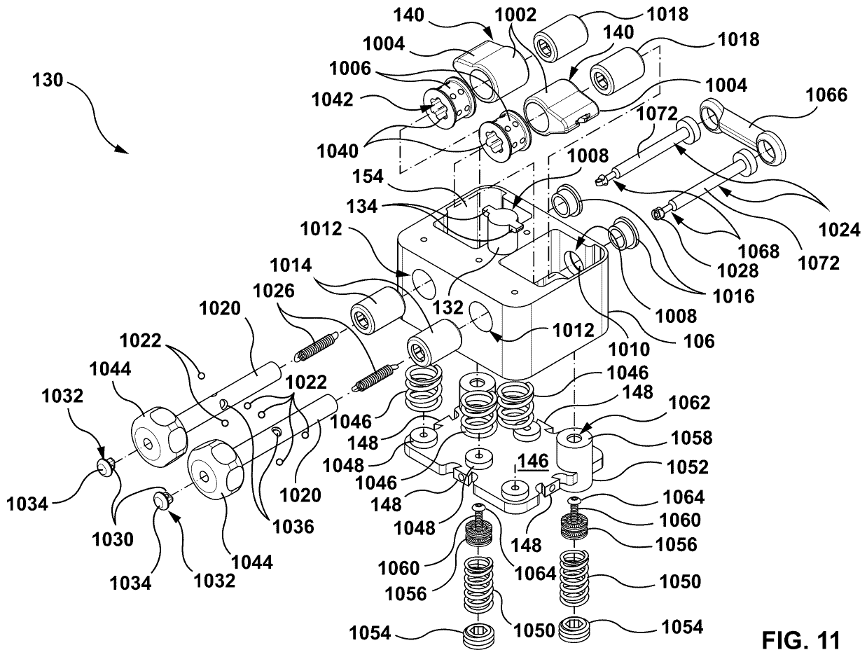

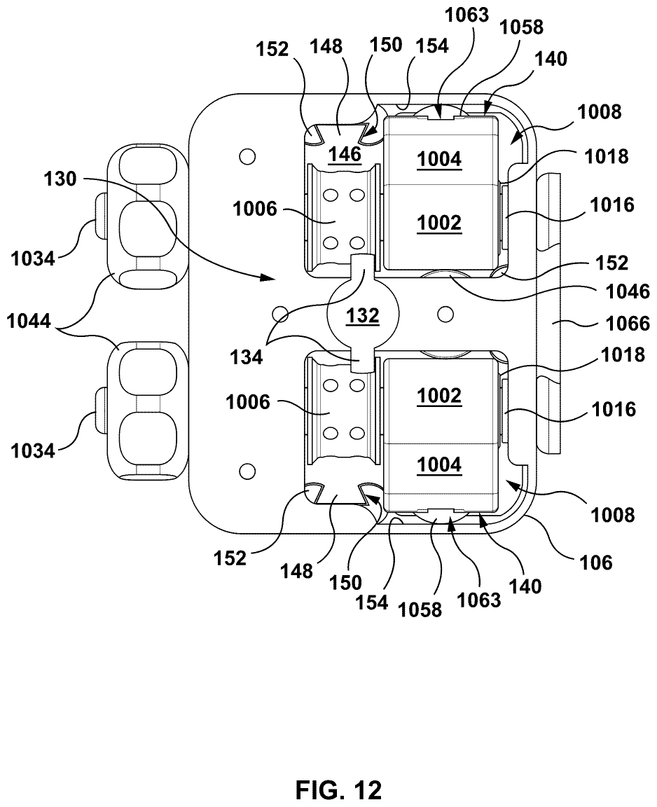

As noted above, in the illustrated embodiment, motion from steps taken with the prosthesis is transmitted to the actuators 140 by a movable platform 146. Referring again to FIGS. 10 and 11, in the illustrated embodiment the movable platform 146 is carried by the housing 102, in particular by the actuator enclosure 106. The four edges of the movable platform 146 each include a recessed dovetail guide follower 148 which is received in a respective correspondingly shaped guide channel 150 (see FIGS. 6, 6C and 12) formed in a respective elongate inward projection 152 on a respective interior sidewall 154 of the actuator enclosure 106 (see FIG. 12). The platform 146 is reciprocally movable toward and away from the residuum receptacle 112 between a distal position (see FIGS. 13A and 13AE) and a proximal position (see FIGS. 13B and 13C). The platform 146 is closer to the residuum receptacle 112 in the proximal position than in the distal position. As can be seen in FIGS. 8A and 8B, the actuators 140 are carried by the housing 102, in particular the actuator enclosure 106, between the residuum receptacle 112 and the platform 146. Thus, in the illustrated embodiment the platform 146 is movable toward and away from the main winch shafts 1020 and the spools 1006, and is closer to the main winch shafts 1020 and the spools 1006 in the proximal position than in the distal position. The platform is biased into the distal position, for example by one or more biasing members.

In the illustrated embodiment, the platform is biased into the distal position by a plurality of cushioning springs 1046 in the form of spaced-apart helical compression springs that are secured on respective locating studs 1048 on the platform 146 and received in corresponding spring recesses in the actuator enclosure 106. Other types of compression springs may also be used. The cushioning springs 1046 may be used, alone or in cooperation with other components, to couple the platform 146 to the actuator enclosure 106. In other embodiments, the platform may be biased into the distal position by a single centrally disposed spring, such as a centrally disposed bellows spring.

Each time a user takes a step with the lower limb prosthesis, when the user puts weight on the end effector (not shown) coupled to the platform 146, the weight will overcome the bias applied by the cushioning springs 1046 and move the platform 146 from the distal position into the proximal position. This movement of the platform 146 in turn transmits movement to a pair of resistive elements 1050 trapped between the platform 146 and the actuators 140, with each resistive element 1050 coupled to the actuator arm 1004 of a respective one of the actuators 140.

In the illustrated embodiment, the resistive elements 1050 are helical compression springs, although this is merely an illustrative example and is not limiting; other types of resistive elements may also be used. The platform 146 carries a pair of opposed hollow cylinder barrels 1052, each of which is positioned in registration with a respective one of the actuator arms 1004. One of the resistive elements 1050 is disposed in each one of the cylinder barrels 1052, and each cylinder barrel 1052 is threaded at its bottom to receive a setscrew 1054 that functions as a cylinder cap. A respective piston 1056 is trapped in each cylinder barrel 1052, between the respective resistive element 1050 and the head 1058 of the respective cylinder barrel 1052 opposite the setscrew 1054. Each piston 1056 carries a piston rod 1060 that projects through a rod aperture 1062 in the head 1058 of the respective cylinder barrel 1052 and terminates in a piston rod connector 1064 which is coupled to a respective one of the actuator arms 1004. In the illustrated embodiment, bolts are used as the piston rods 1060 with the bolt heads functioning as the piston rod connectors 1064. The actuator arms 1004 each include an elongate T-shaped slot 1063 that receives a respective one of the piston rod connectors 1064 such that axial movement of the piston 1056 will cause the respective piston rod 1060 to pull or push the actuator arm 1004. Thus, movement of the piston 1056 and with it the piston rod 1060 can drive movement of the respective actuator arm 1004 between the rest position (FIGS. 13A and 13AE) and the actuated position (FIGS. 13B and 13BE). The use of bolts as piston rods is merely an illustrative embodiment and is not limiting. For example, and without limitation, in other embodiments, the piston rod connector may be a clevis assembly, or a hook, or an eye. The coupling of the piston rod connectors 1064 to the actuator arms 1004, in cooperation with the cushioning springs 1046, couples the platform 146 to the actuator enclosure 106. Other couplings between the platform 146 and the actuator enclosure 106 are also contemplated.

Reference is now made to FIGS. 13A through 13C and 13AE through 13CE. FIGS. 13A and 13AE show the actuator arms 1004 in the rest position with the platform 146 in the distal position; FIGS. 13B and 13BE show the actuator arms 1004 in the actuated position with the platform 146 in the proximal position, and FIGS. 13C and 13CE show the actuator arms 1004 in the rest position with the platform 146 in the proximal position.

FIGS. 13A and 13AE represent the configuration of the winch assembly 130 when a user is not applying weight to the end effector, for example when the user is walking and the end effector has yet to engage the surface (e.g. ground or floor) upon which the user is walking, or if the user's weight is on the alternate lower limb. When the user applies weight to the end effector, the winch assembly 130 will move to either the configuration in FIGS. 13B and 13BE, or to the configuration in FIGS. 13C and 13CE, depending on the tension in the cables 120. When the user applies weight to the end effector, the force applied by the weight of the user overcomes the resistance of the cushioning springs 1046, causing the platform 146 to move from the distal position (FIGS. 13A and 13AE) into the proximal position (FIGS. 13B and 13BE and FIGS. 13C and 13CE). This movement of the platform 146 applies force to the resistive elements 1050 via the setscrews 1054, since each resistive element 1050 is trapped in the respective cylinder barrel 1052 between the respective setscrew 1054 and the respective piston 1056, which is movable within the respective cylinder barrel 1052. As the platform 146 moves from the distal position (FIGS. 13A and 13AE) into the proximal position (FIGS. 13B and 13BE and FIGS. 13C and 13CE), the respective resistive element 1050 will either act as a rigid body bracing the respective piston 1056 against the head 1058 of the respective cylinder barrel 1052 (FIGS. 13B and 13BE), or yield against the respective piston 1056 (FIGS. 13C and 13CE), depending on whether the resistance to movement of the respective piston 1056 is greater than the resistance to further compression of the respective resistive element 1004. Because the respective piston 1056 engages the respective actuator arm 1004, the resistance to movement of the respective piston 1056 depends on the resistance to movement of the respective actuator arm 1004. Movement of the respective actuator arm 1004 rotates the respective actuator body 1002, which in turn rotates the main winch shaft 1020 and the spool 1006 to further wind the respective cable 120 onto the spool 1006 to incrementally increase the tension in the respective cable 120. Therefore, the resistance to movement of the respective piston 1056 depends on the tension in the respective cable(s) 120.

More particularly, the respective piston 1056 cannot move with the platform 146 as the platform 146 moves into the proximal position unless the respective actuator arm 1004 can move into the actuated position by rotating the respective actuator body 1002 to pivot the respective actuator 140. But the respective actuator 140 is bound to the respective main winch shaft 1020 by the respective one-way actuator needle bearing 1018, so the respective actuator 140 cannot pivot unless the respective main winch shaft 1020 can rotate. Since the respective main winch shaft 1020 is fixed to the spool 1006, the respective main winch shaft 1020 cannot rotate unless the spool 1006 can also rotate. Since the respective cable 120 is wound onto the respective spool 1006 and is under tension, rotation of the respective spool 1006 requires that the existing tension in the respective cable 120 be overcome. Thus, the tension in the respective cable 120 propagates back through the respective spool 1006, main winch shaft 1020 and actuator needle bearing 1018 to resist pivoting of the respective actuator 140, thereby resisting movement of the actuator arm 1004 from the rest position to the actuated position and providing the resistance to movement of the respective piston 1056.

Because the tension in the cables 120 tightens the panels 116 against the residuum, the amount of tension in the cables 120 corresponds to the tightness of the retention mechanism comprising the panels 116. If the tension in the cables 120 is below a threshold, when the end effector engages the surface, the winch assembly 130 will move to the configuration in FIGS. 13B and 13BE, which will increase tension in the cables 120. However, when the tension in the cables 120 is at or above the threshold, when the end effector engages the surface the winch assembly 130 will move to the configuration in FIGS. 13C and 13CE, which will not increase the tension in the cables 120. The threshold for the tension in the cables 120 corresponds to the resistance of the resistive elements 1050, which may be adjusted by tightening or loosening the respective setscrew 1054 so that the threshold is adjustable. Moreover, the resistance of the respective resistive elements 1050 need not be identical.

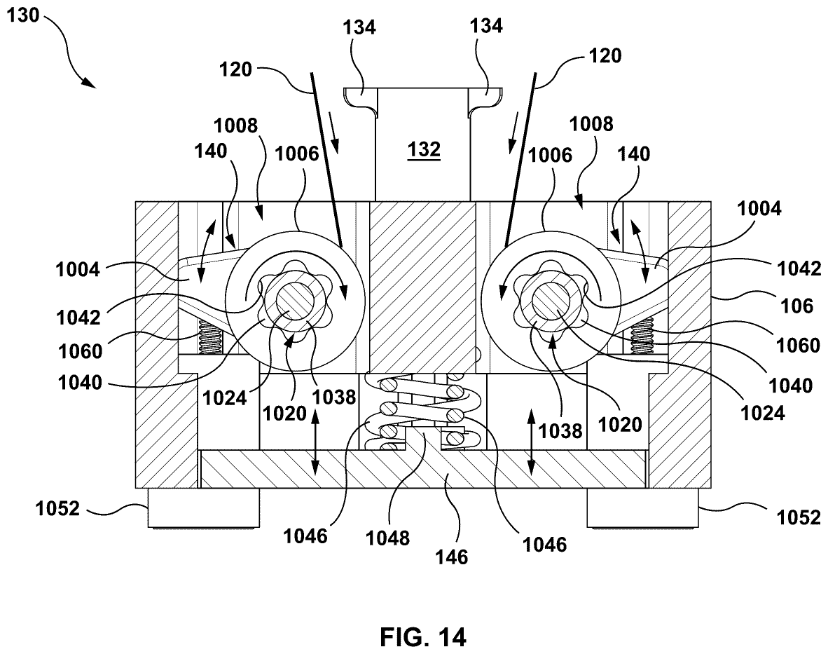

Reference is first made to FIGS. 13B and 13BE, which illustrate the scenario where the tension in the cables 120 is below the threshold. In other words, in the scenario shown in FIGS. 13B and 13BE, the amount of force required to further compress the respective resistive element 1050 exceeds the resistance to movement of the respective piston 1056 resulting from the tension in the corresponding cable 120. Therefore, the respective resistive element 1050 does not compress, and instead acts as a rigid body that braces the respective piston 1056 against the setscrew 1054, so that movement of the platform 146 toward the actuator 140 will drive the piston rod 1060 into the actuator arm 1004 to move the actuator arm 1004 into the actuated position and rotate the actuator body 1002. This in turn rotates the main winch shaft 1020 and the spool 1006 to further wind the respective cable 120 onto the spool 1006 to incrementally increase the tension in the respective cable 120. Thus, where the tension in the cables 120 is below the threshold, movement of the platform 146 toward the proximal position pushes the respective resistive element 1050 toward the actuator 140 whereby the respective resistive element 1050 transmits the movement of the platform 146 via the piston 1056 and piston rod 1060 to the actuator arm 1004 to pivot the actuator 140 and thereby index the spool 1006. Accordingly, when the tightness of the retention mechanism comprising the panels 116 and cables 120 is below a desired threshold, each step transmits motion across the resistive elements 1050 to the actuators 140 to cycle the actuators 140. FIG. 14 shows how each movement of the respective actuator arm 1004 into the actuated position rotates the respective actuator body 1002, which (via the actuator needle bearing 1018, not shown in FIG. 14) rotates the respective main winch shaft 1020 and thereby rotates the respective spool 1006 to wind the respective cable 120 onto the spool 1006 to incrementally increase the tension in the cable 120.

Reference is now made to FIGS. 13C and 13CE, which illustrate the scenario where the tension in the cables 120 is at or above the threshold. In the scenario shown in FIGS. 13C and 13CE, the amount of force required to further compress the respective resistive element 1050 is less than the resistance to movement of the respective piston 1056 resulting from the tension in the corresponding cable 120 transmitted back to the respective actuator arm 1004. In other words, more force is required to overcome the tension in the respective cable 120 and move the actuator arm 1004 from the rest position to the actuated position than is required to further compress the respective resistive element 1050. Therefore, instead of movement of the platform 146 being transmitted through the respective resistive element 1050 to move the respective piston 1056 and pivot the respective actuator 140, the respective resistive element 1050 acts like a spring rather than a rigid body, and is compressed between the respective setscrew 1054 and the respective piston 1056 as the platform 146 moves. In other words, the resistance of the respective resistive element 1050 cannot overcome the tension in the respective cable 120, so the resistive element 1050 yields (e.g. the spring is compressed). Accordingly, instead of the piston rod 1060 moving the actuator arm 1004 toward the actuated position, the actuator arm 1004 is maintained in position by the tension in the cable 120 and the respective piston 1056 is pushed into the cylinder barrel 1052, where it compresses the respective resistive element 1050 against the respective setscrew 1054. Thus, when the tightness of the retention mechanism comprising the panels 116 has reached the threshold, on each further step the resistive elements 1050 yield to absorb the motion of the platform 146, rather than transmitting the motion of the platform 146 to the actuators 140. When the resistive elements 1050 yield instead of transmitting motion, the actuators 140 will fail to cycle on each further step, thereby inhibiting further tightening of the retention mechanism beyond the threshold.

When the user removes the weight from the end effector, the cushioning springs 1046 return the platform 146 from the proximal position (FIGS. 13B and 13BE and FIG. 13CE) to the distal position (FIGS. 13A and 13AE).

Accordingly, as has been shown with respect to FIGS. 13A through 13C and FIG. 13AE through 13CE, reciprocal movement of the platform 146 into the proximal position and back to the distal position cycles the actuators 140 only where the resistance to compression of the respective resistive element 1050 exceeds the resistance to movement of the respective actuator 140 resulting from the tension in the respective cable 120 so that the respective resistive element 1050 transmits the movement of the platform 146 to the respective actuator 140 instead of yielding to the movement of the platform 146.

Thus, in the illustrated embodiment, the resistive element 1050, actuator 140 and actuator needle bearing 1018 provide a mechanical interface to transmit movement of the end effector of the lower limb prosthesis toward the residuum from a step to a tensioner comprising the main winch shaft 1020, release shaft 1024, locking ball bearings 1022, and spool 1006, with resistance of the mechanical interface provided by the resistive element 1050 (e.g. a spring).

As noted above, in a preferred embodiment the locking mechanism is a releasable locking mechanism. Reference is now made to FIGS. 15A and 15B, which show an illustrative implementation of a releasable locking mechanism. In the illustrated embodiment, each spool 1006 is coupled to its respective main winch shaft 1020 by way of interengagement between the locking ball bearings 1022, the main winch shaft 1020, and the release shaft 1024 disposed concentrically within the lumen of the hollow main winch shaft 1020. Each release shaft 1024 is axially movable within its respective main winch shaft 1020, and is coupled to the main winch shaft 1020 by the release spring 1026 as described above, and the locking ball bearings 1022 are received within respective locating apertures 1036 extending through the annular wall 1038 of the respective main winch shaft 1020. When the release shaft 1024 is in a locking position relative to the main winch shaft 1020, as shown in FIG. 15A, the locking ball bearings 1022 are forced outwardly by the release shaft 1024 and engage with the locking channels 1040 in the inner surface 1042 of the respective spool 1006 so that rotation of the respective main winch shaft 1020 will result in rotation of the respective spool 1006. Conversely, the respective spool 1006 cannot rotate unless the respective main winch shaft 1020 also rotates, but such rotation in the unwinding direction of the respective spool 1006 is resisted by the respective winch needle bearing 1014 so that when the release shaft 1024 is in the locking position, tension on the respective cable 120 is maintained.

Reference is now made to FIG. 15B. In the illustrated embodiment, the release shaft 1024 can be moved axially relative to its respective main winch shaft 1020 by pulling on a release handle 1066 coupled to the ends of the release shafts 1024 opposite the ends having the eyes 1028. This extends the release spring 1026 which, when the force applied to the release handle 1066 is released, will return the release shaft 1024 to the locking position. Thus, in the illustrated embodiment the release shaft 1024 is biased into the locking position. By moving the release shaft 1024 axially relative to its respective main winch shaft 1020, the locking ball bearings 1022 can be disengaged from the locking channels 1040 in the inner surface 1042 of the respective spool 1006 so the respective spool 1006 can rotate freely in either direction relative to the respective main winch shaft 1020. This allows the respective spool 1006 to rotate relative to the respective main winch shaft 1020 in the unwinding direction to slacken the respective cable 120 and release tension therein. More particularly, in the illustrated embodiment, by moving the respective release shaft 1024 axially away from the manual tensioning knob 1044 of the respective main winch shaft 1020, an annular recess 1068 on the release shaft 1024 can be brought into registration with the locating apertures 1036 in the annular wall 1036 in the respective main winch shaft 1038. This is the release position of the release shaft 1024, relative to the respective main winch shaft 1038. When the annular recess 1068 on the release shaft 1024 is in registration with the locating apertures 1036 in the annular wall 1036 in the respective main winch shaft 1038, the locking ball bearings 1022 can move radially inwardly into the annular recess 1068, away from the inner surface 1042 of the spool 1006, while remaining trapped in the locating apertures 1036. This disengages the locking ball bearings 1022 from the locking channels 1040, allowing the respective spool 1006 to rotate freely relative to the respective main winch shaft 1038. The spring 1026 will return the release shaft 1024 to the locking position when the user lets go of the release handle 1066. As the release shaft 1024 moves axially back to the locking position (FIG. 15A), a beveled edge 1070 of the annular recess 1068 guides the locking ball bearings 1022 radially outwardly back onto the main body 1072 of the release shaft 1024. This moves the locking ball bearings 1022 radially outwardly through the locating apertures 1036 and back into engagement with the locking channels 1040 in the inner surface 1042 of the spool 1006. While the illustrative embodiment uses a single release handle 1066 for both release shafts 1024, other embodiments may provide for the release shafts to be manipulated individually.

In the illustrative embodiment, the winch assembly 130 comprises two actuators 140, which wind two cables 120 onto two spools 1006. It is contemplated that in other embodiments there may be a single actuator, or more than two actuators, and that there may be a single spool winding a single cable, or more than two spools winding more than two cables.

FIG. 16 shows another non-limiting illustrative embodiment of a self-adjusting socket 1600 for a lower limb prosthesis. The illustrative socket 1600 is also for a transtibial prosthesis, but can be adapted for use with a socket for a transfemoral amputee by one of ordinary skill in the art, now informed by the present disclosure. The self-adjusting socket 1600 comprises a housing 1602, which in turn comprises a receptacle body 1604 and an actuator enclosure 1606 carried by the receptacle body 1604. The receptacle body 1604 is generally hollow with an open end 1608 and a support end 1610 opposite the open end 1608, and the interior volume of the receptacle body 1604 forms a residuum receptacle 1612 adapted to receive the residuum of an amputee via the open end 1608. Thus, the housing 1602, in particular the receptacle body 1604 thereof, comprises a residuum receptacle 1612. An end effector 1615 is coupled to the socket 1600 via an actuator enclosure 1606 that is coupled to the receptacle body 1604.

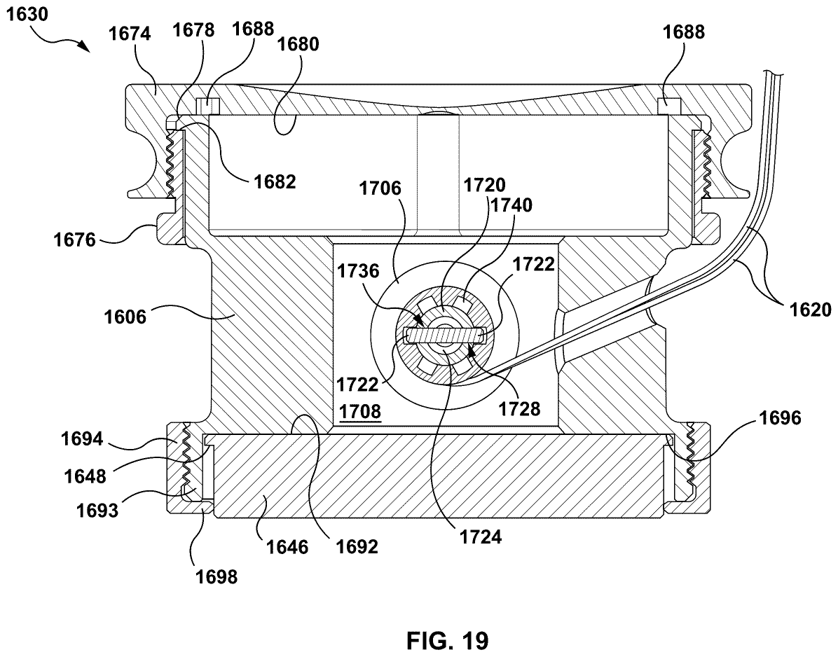

The housing 1602 carries a retention mechanism which, in the illustrated embodiment, comprises three panels 1616 arranged circumferentially about the residuum receptacle 1612 and disposed in respective openings 1618 in the receptacle body 1604 so as to be inwardly and outwardly displaceable relative to the receptacle body 1604 of the housing 1602. This is merely a non-limiting illustrative embodiment; more or fewer panels may be present, or an alternate retention mechanism may be used. The panels 1616 may include interiorly facing pads for cushioning. A single looped cable 1620 (FIG. 19) runs through tubular cable guides 1622 formed on the panels 1616 and through covered cable guides 1624 on the outer surface of the receptacle body 1604, with the ends of the cable 1620 being coupled to a winch assembly 1630, which is described further below. By increasing and decreasing the tension in the cable 1620, the tightness of the retention mechanism can be adjusted.

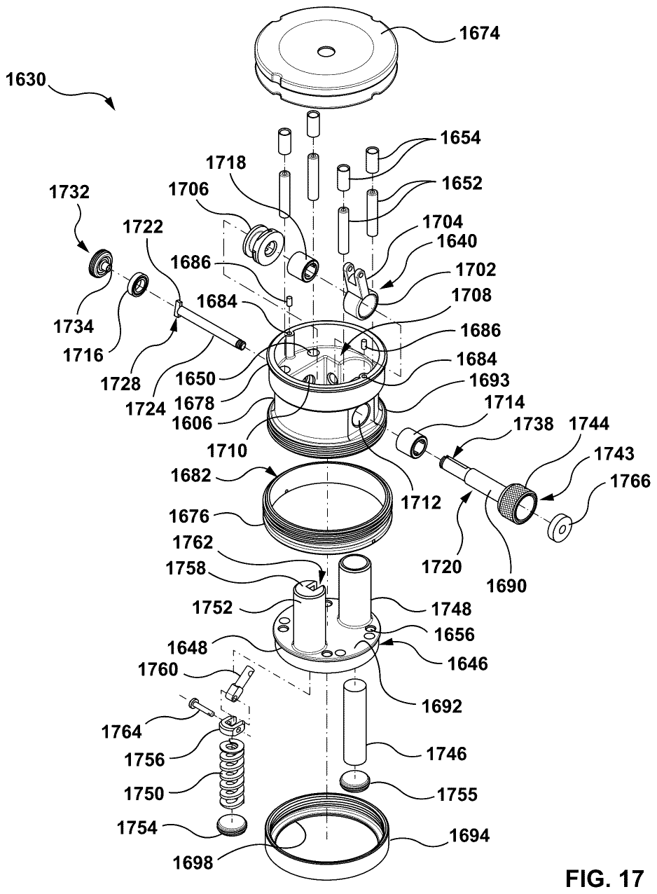

FIGS. 17 to 21B show another illustrative winch assembly 1630, which may be used as part of a socket adjustment mechanism, for example as part of the self-adjusting socket 1600 shown in FIG. 16, where the actuator enclosure 1606 is connectable to the residuum receptacle 1612, specifically to the receptacle body 1604, and functions as a winch body that carries the components of the winch assembly 1630.

Reference is now made to FIG. 17. A generally circular lamination plate 1674 can be integrated into a lower limb prosthesis in known manner, for example by lamination as a prosthetist builds up the layers of the part of the prosthesis that will form the residuum receptacle, and thereby connected to the receptacle body 1604 (FIG. 16). The lamination plate 1674 is internally threaded, and can threadedly receive an externally threaded adjustment mechanism retaining ring 1676. A proximal end of the actuator enclosure 1606 has an outwardly projecting annular enclosure flange 1678 which is captured between the distal surface 1680 (FIG. 19) of the lamination plate 1674 and the proximal surface 1682 of the adjustment mechanism retaining ring 1676 to releasably connect the actuator enclosure 1606 to the receptacle body 1604 forming the residuum receptacle 1612. Two diametrically opposed locating pin apertures 1684 are also disposed at the proximal end of the actuator enclosure 1606; locating pins 1686 are friction fit into the locating pin apertures 1684. These locating pins 1686 can in turn be received in one of a plurality of circumferentially extending locating apertures 1688 (FIG. 19) in the distal surface 1680 of the lamination plate 1674 to selectively fix the rotational position of the actuator enclosure 1606 relative to the lamination plate 1674. The embodiment shown and described is merely an illustrative, non-limiting example of how an actuator enclosure 1606 may be connectable to a residuum receptacle.

In the illustrated embodiment, the winch assembly 1630 comprises a single actuator 1640, which is disposed within the actuator enclosure 1606. The actuator 1640 comprises a rocker having a tubular cylindrical actuator body 1702 and an outwardly extending forked actuator arm 1704 that acts as a lever to pivot the actuator 1640 so that the actuator 1640 can rock back and forth about an axis extending through the actuator body 1702. Thus, the actuator 1640 is configured to reciprocally cycle between a rest position and an actuated position, as described further below. This is merely one non-limiting illustrative embodiment of an actuator, and other types of actuators are also contemplated.

Motion from steps taken with the prosthesis is transmitted to the actuator 1640 by a movable platform 1646 coupled to the end effector 1615 (FIG. 16). The end effector 1615 shown is merely illustrative, and is not limiting, any suitable type of end effector may be used and an end effector may be coupled to the movable platform by any suitable technique. In the illustrated embodiment the movable platform 1646 is carried by the actuator enclosure 1606. The movable platform 1646 comprises a platform base 1692 that supports a hollow cylinder barrel 1752 and a hollow cushioning element guide tube 1748, which are slidably received in respective penannular recesses formed in the winch cavity 1708. A plurality of platform guide channels 1650 are formed through the actuator enclosure 1640, outside of the winch cavity 1708. Platform bushings 1654 (bearings may be used instead) are friction fit into the platform guide channels 1650. Platform guide rods 1652 are fixed (e.g. friction fit or threaded) into corresponding guide rod holes 1656 in the platform base 1692 in registration with the platform guide channels 1650. The platform guide rods 1652 are slidingly received in the platform bushings 1654 within the platform guide channels 1650 so that the movable platform 1646 can move axially relative to the actuator enclosure 1640. The movable platform 1646 is coupled to the actuator enclosure 1606 by an internally threaded platform retainer ring 1694 that is received on the outwardly threaded outwardly necked distal end 1693 of the actuator enclosure 1606. The outwardly necked distal end 1693 of the actuator enclosure 1606 forms an inner shoulder 1696 (see FIG. 19), and the platform retainer ring 1694 has an inwardly projecting annular platform retainer flange 1698. The platform base 1692 is circumvallated by an outwardly projecting annular platform flange 1648, which is trapped between the inner shoulder 1696 on the actuator enclosure 1606 and the annular platform retainer flange 1698 on the platform retainer ring 1694 (see FIG. 19). This allows the platform to reciprocate between a proximal position and a distal position.

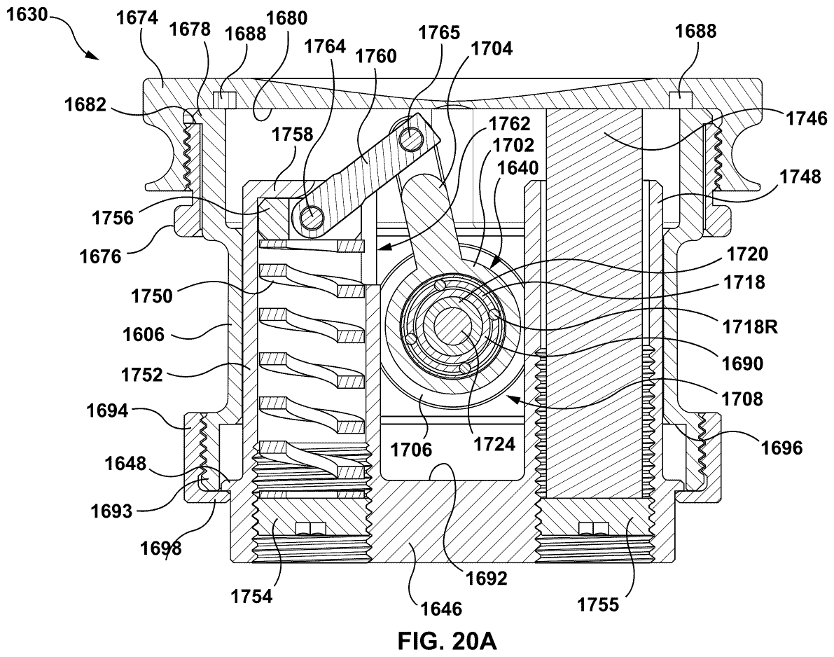

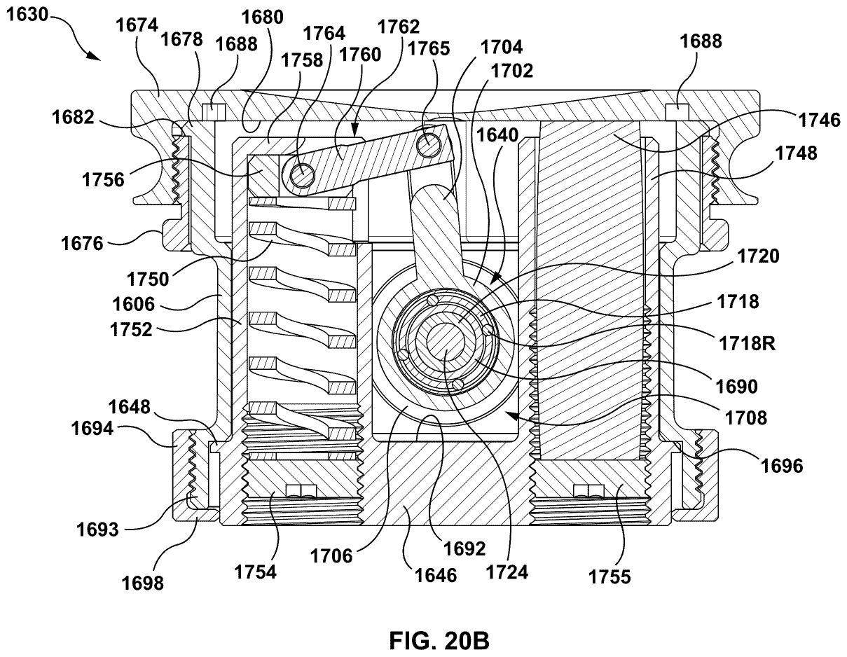

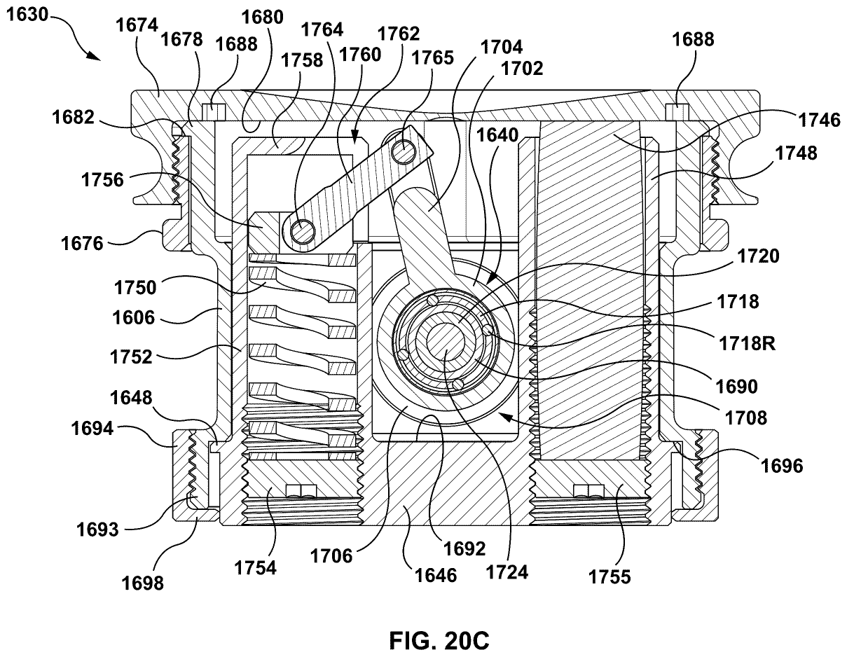

A resistive element 1750 is disposed in the cylinder barrel 1752, and the cylinder barrel 1752 is internally threaded at its distal end to receive a cylinder setscrew 1754 that functions as a cylinder cap. In the illustrated embodiment the resistive element 1750 is a compression spring, but this is merely illustrative and not limiting. A piston 1756 is trapped in the cylinder barrel 1752 between the resistive element 1750 and the head 1758 of the cylinder barrel 1752 opposite the cylinder setscrew 1754. The piston 1756 is pivotally coupled by a piston connector pin 1764 to a piston arm 1760 that projects through an articulation aperture 1762 in the cylinder barrel 1752 and head 1758 and is pivotally coupled to the forked end of the actuator arm 1704 by an actuator connector pin 1765 (see FIGS. 20A to 20C). Axial movement of the piston 1756 will cause the piston arm 1760 to pull or push the actuator arm 1704. Thus, movement of the piston 1756 and with it the piston arm 1760 can drive movement of the actuator arm 1704 between the rest position (FIG. 20A) and the actuated position (FIG. 20B).

In the illustrated embodiment, the movable platform 1646 is biased into the distal position by a resilient cushioning element 1746 in the form of a urethane rod that is received in the cushioning element guide tube 1748 and trapped therein between the distal surface 1680 of the lamination plate 1674 and a guide tube setscrew 1755 threaded into a corresponding aperture in the platform base 1692. Thus, the resilient cushioning element 1746 acts between the lamination plate 1674 and the platform base 1692. A urethane rod is merely an illustrative, non-limiting embodiment of one type of cushioning element. For example, a compression spring could be used as a cushioning element instead. Moreover, in an alternate embodiment, a urethane rod may be used as the resistive element.

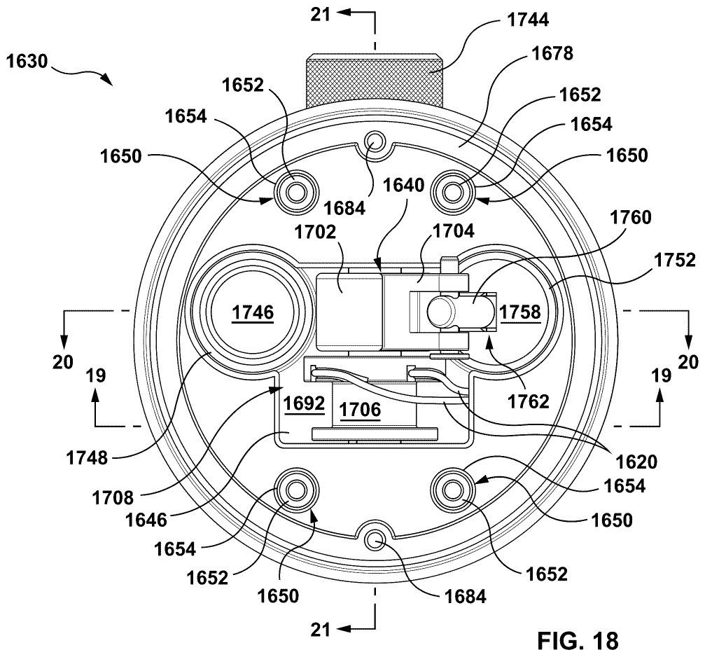

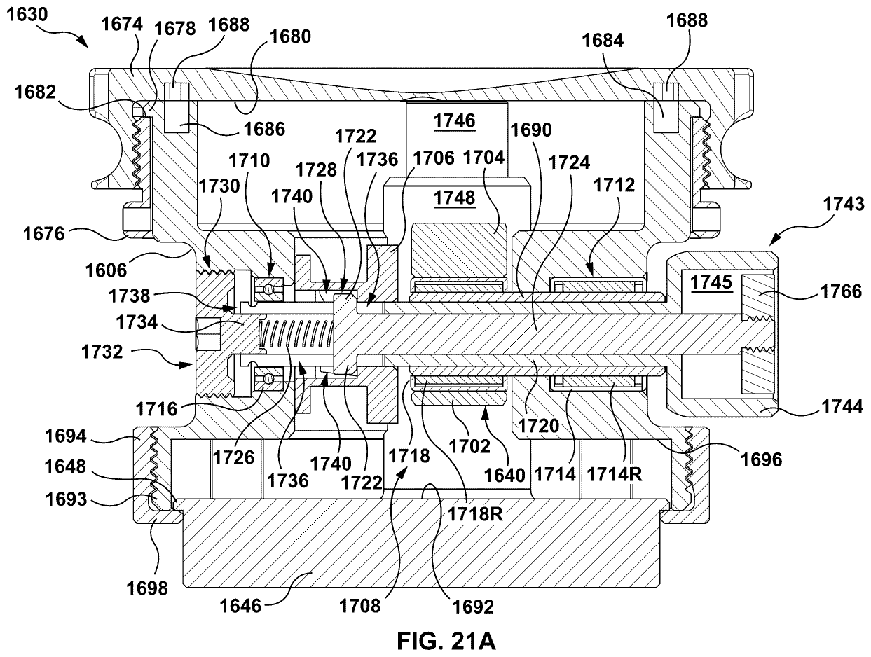

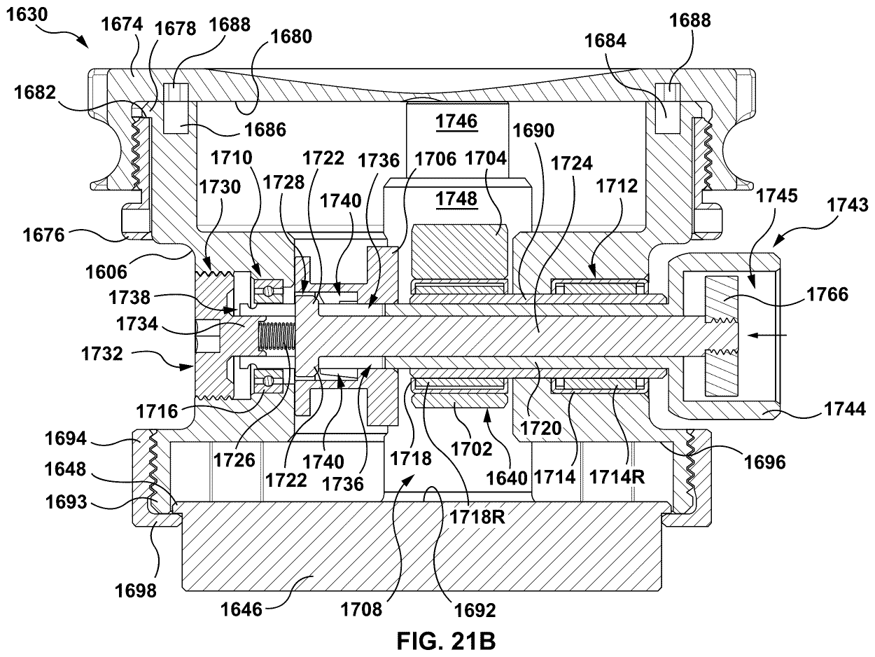

With reference now to FIGS. 21A and 21B, the actuator 1640 is coupled to a spool 1706, as described in more detail below. The actuator 1640 is connectable to a residuum retention mechanism via the spool 1706, onto which cables 1620 (see FIGS. 18 and 19) may be wound and from which the cables may be unwound to respectively tighten and loosen the retention mechanism. The actuator 1640 and the spool 1706 are disposed in a winch cavity 1708 formed within the actuator enclosure 1606, which also includes a plurality of bearing apertures. A first bearing aperture 1710 is disposed at one end of the winch cavity 1708 and a second bearing aperture 1712 is disposed at the opposite end of the winch cavity 1708, in registration with one another. A winch roller bearing 1716 is fitted into the first bearing aperture 1710, and a winch needle bearing 1714 with winch needle bearing rollers 1714R is friction fit into the second bearing aperture 1712, so that the winch needle bearing 1714 and winch roller bearing 1716 are arranged coaxially with one another. The winch needle bearing 1714 is a one-way needle bearing, and the winch roller bearing 1716 is a two-way ball bearing, although other embodiments may use a one-way bearing that cooperates with the winch needle bearing 1714. Optionally, the winch roller bearing 1716 may be replaced with a bushing.

In the illustrated embodiment, the actuator 1640 is releasably coupled to the spool 1706 by way of an actuator needle bearing 1718, a hollow main winch shaft 1720, and a release shaft 1724 disposed concentrically within the lumen of the hollow main winch shaft 1720. The actuator needle bearing 1718 is a one-way needle bearing including actuator needle bearing rollers 1718R and which is friction fit into the opening of the tubular cylindrical actuator body 1702. The main winch shaft 1720 passes through the winch roller bearing 1716, actuator needle bearing 1718, and winch needle bearing 1714. In the illustrated embodiment, a friction sleeve 1690 is friction fit over the body of the main winch shaft 1720, with the portion including the friction sleeve 1690 passing through the actuator needle bearing 1718 and the winch needle bearing 1714. The actuator needle bearing 1718 and the winch needle bearings 1714 are arranged so that the actuator needle bearing 1718 and the winch needle bearing 1714 have a common permitted direction of rotation.

The release shaft 1724 is axially movable within the main winch shaft 1020. The end 1728 of the release shaft 1724 closest to the winch roller bearing 1716 is T-shaped. Crossbar arms 1722 of the T-shaped end 1728 extend through diametrically opposed axially extending slots 1736 formed through an inwardly necked spool end 1738 of the main winch shaft 1720. In the illustrated embodiment, termination of the friction sleeve 1690 distal from a manual tensioning knob 1744 disposed at a knob end 1743 of the main winch shaft 1720 produces the inward necking of the spool end 1738 of the main winch shaft 1720 although other arrangements are also contemplated. Thus, when the release shaft 1724 moves axially within the main winch shaft 1020, the crossbar arms 1722 move axially within the slots 1736. A biasing member, in this case a helical compression spring which serves as a release spring 1726, is received coaxially within the spool end 1738 of the main winch shaft 1720. A spring retainer 1732 is threaded into a corresponding retainer aperture 1730 in the actuator enclosure 1606, with the retainer aperture 1730 being in concentric registration with the first bearing aperture 1710 so that the spring retainer 1732 is in concentric registration with the winch roller bearing 1716. A central post 1734 of the spring retainer 1732 extends into the spool end 1738 of the main winch shaft 1720, and the release spring 1726 is trapped inside the hollow spool end 1738 of the main winch shaft 1720, between the central post 1734 of the spring retainer 1732 and the T-shaped end 1728 of the release shaft 1724.

The spool 1706 is received on the spool end 1738 of the main winch shaft 1720. The crossbar arms 1722 at the T-shaped end 1728 of the release shaft 1724 project through the slots 1736 at the spool end 1738 of the main winch shaft 1720, and can extend into corresponding locking channels 1740 formed inside the spool 1606. When the crossbar arms 1722 extend into the locking channels 1740, rotation of the main winch shaft 1620 will result in rotation of the spool 1706.

The main winch shaft 1720 terminates with a manual tensioning knob 1744 at a knob end 1743 of the main winch shaft 1720 opposite the spool end 1738 that receives the spring retainer 1732. Preferably the manual tensioning knob 1744 is monolithically formed as part of the main winch shaft 1720; in other embodiments it may be a separate part affixed to the main winch shaft. In the illustrated embodiment, the manual tensioning knob 1744 is annular and forms a cavity 1745 in which a release button 1766 may be nested, as described further below. By rotating the manual tensioning knob 1744 in the permitted direction of rotation, the main winch shaft 1720 and hence the spool 1706 may be rotated independently of the actuator 1640 to manually apply tension to the cables. Thus, the manual tensioning knob 1744 provides a manual tightening mechanism for tightening the retention mechanism.

Continuing to refer to FIGS. 21A and 21B, release of the releasable locking mechanism will now be described. As noted above, in the illustrated embodiment, the spool 1706 is coupled to the main winch shaft 1720 by way of the crossbar arms 1722 of the T-shaped end 1728 of the release shaft 1724 disposed concentrically within the lumen of the hollow main winch shaft 1720. The crossbar arms 1722 extend through the slots 1736 in spool end 1738 of the main winch shaft 1720, and when the release shaft 1724 moves axially within the main winch shaft 1020, the crossbar arms 1722 move axially within the slots 1736. The release spring 1726 acts between the central post 1734 of the spring retainer 1732 and the T-shaped end 1728 of the release shaft 1724 to urge the release shaft 1724 away from the spring retainer 1732. This urges the release shaft 1724 toward a locking position relative to the main winch shaft 1720, as shown in FIG. 20A, where the T-shaped end 1728 of the release shaft 1724 is in axial registration with the locking channels 1740 inside the spool 1006 so that the crossbar arms 1722 extend through the slots 1736 into the locking channels 1740. With the release shaft 1724 in the locking position, rotation of the main winch shaft 1720 will result in rotation of the spool 1706. Conversely, the spool 1706 cannot rotate unless the main winch shaft 1720 also rotates. When the release shaft 1724 is in the locking position, such rotation in the unwinding direction of the spool 1706 is resisted by the winch needle bearing 1714 so that tension on the cable 1620 is maintained.

Reference is now made to FIG. 21B. In the illustrated embodiment, the release shaft 1724 can be moved axially relative to the main winch shaft 1720 by pushing on the release button 1766 and moving it into the cavity 1745 within the manual tensioning knob 1744. Since the release button 1766 is coupled (e.g. threaded as shown) to the end of the release shaft 1724 opposite the T-shaped end 1728, pushing the release button 1766 compresses the release spring 1726 which, when the force applied to the release button 1766 is released, will return the release shaft 1724 to the locking position. Thus, in the illustrated embodiment the release shaft 1724 is biased into the locking position. Using the release button 1766, the release shaft 1724 can be moved axially relative to the main winch shaft 1720 toward the spring retainer 1732, placing the release shaft in a release position in which the T-shaped end 1728 has been moved out of axial registration with the locking channels 1740. This disengages the crossbar arms 1722 from the locking channels 1740 in the spool 1706 so the spool 1706 can rotate freely in either direction relative to the main winch shaft 1720. This allows the spool 1706 to rotate relative to the main winch shaft 1720 in the unwinding direction to slacken the cable 1620 and release tension therein. The release spring 1726 will return the release shaft 1724 to the locking position when the user relents from pressing the release button 1766, moving the T-shaped end 1728 back into axial registration with the locking channels 1740 and thereby moving the crossbar arms 1722 along the slots 1736 back into engagement with the locking channels 1740.

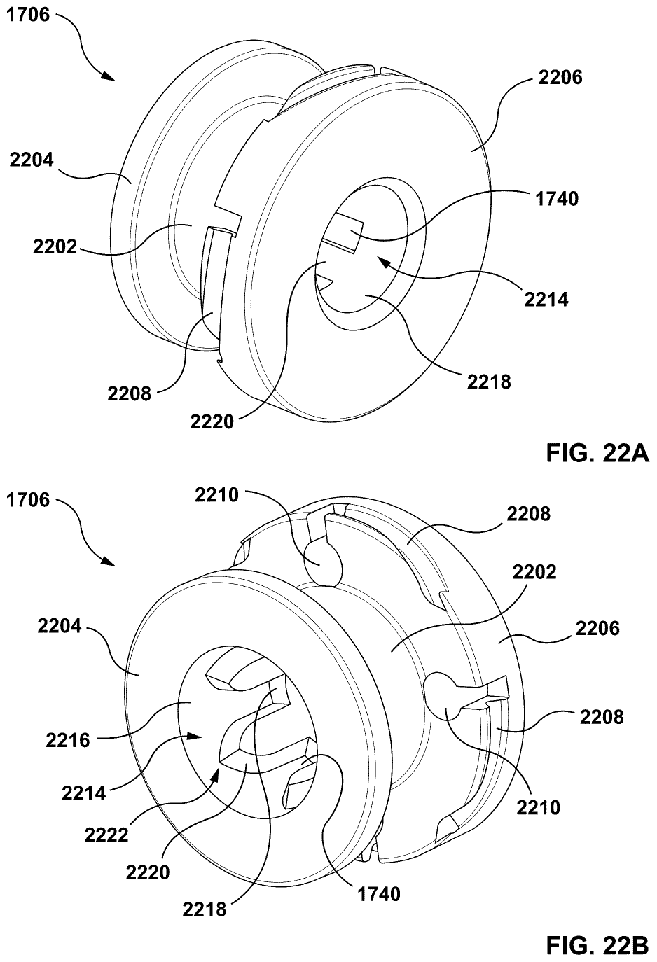

FIGS. 22A and 22B show the spool 1706 in isolation. The spool comprises a barrel 2202 around which the cable 1620 may be wound, with flanges 2204, 2206 disposed at either end of the barrel 2202. One of the flanges 2206 is a cable-retaining flange 2206, and includes spaced-apart circumferentially extending cable grooves 2208 terminating in respective cable anchor wells 2210. An arbor hole 2214 extends through the barrel 2202 and flanges 2204, 2206. The surface 2216 of the arbor hole 2214 has a crenate ring 2218 formed thereon adjacent the cable-retaining flange 2206; the crenate ring 2218 includes a plurality of spaced-apart gladiate elements 2220 extending toward the other flange 2214 with the locking channels 1740 formed by the spaces between the gladiate elements 2220. The tapered ends 2222 of the gladiate elements 2220 assist in guiding the crossbar arms 1722 into the locking channels 1740.

Each time a user takes a step with the lower limb prosthesis, when the user puts weight on the end effector 1615 (FIG. 16) coupled to the platform 1646, the weight will overcome the bias applied by the cushioning element 1746 and move the platform 1646 from the distal position into the proximal position. This movement of the platform 1646 in turn transmits movement to the resistive element 1750 trapped between the platform base 1692 (specifically the cylinder setscrew 1754) and the piston 1756, with the resistive element 1750 coupled to the actuator arm 1704 via the piston 1756 and piston arm 1760.

Reference is now made to FIGS. 20A through 20C. In the description that follows, the terms “rest position” and “actuated position” are applied to the actuator arm 1704 since the relative position of the actuator arm 1704 is clearly visible in the drawings. It will be understood that when the actuator arm 1704 is in the rest position the actuator 1640 of which the actuator arm 1704 forms a part will likewise be in the rest position, and conversely when the actuator arm 1704 is in the actuated position the actuator 1640 of which the actuator arm 1704 forms a part will likewise be in the actuated position. FIG. 20A shows the actuator arm 1704 in the rest position with the platform 1646 in the distal position, FIG. 20B shows the actuator arm 1704 in the actuated position with the platform 1646 in the proximal position, and FIG. 20C shows the actuator arm 1704 in the rest position with the platform 1646 in the proximal position.

FIG. 20A represents the configuration of the winch assembly 1630 when a user is not applying weight to the end effector 1615. When the user applies weight to the end effector 1615, the winch assembly 1630 will move either to the configuration in FIG. 20B, or to the configuration in FIG. 20C, depending on the tension in the cables 1620. When the user applies weight to the end effector 1615, the force applied by the weight of the user overcomes the resistance of the cushioning element 1746, moving the platform 1646 from the distal position (FIG. 20A) into the proximal position (FIGS. 20B and 20C). This movement of the platform 1646 applies force to the resistive element 1750 via the cylinder setscrew 1754, since the resistive element 1750 is trapped in the cylinder barrel 1752 between the cylinder setscrew 1754 and the piston 1756, which is movable within the cylinder barrel 1752. As the platform 1646 moves from the distal position (FIG. 20A) into the proximal position (FIG. 20B and FIG. 20C), the resistive element 1750 will either act as a rigid body bracing the piston 1756 against the head 1758 of the cylinder barrel 1752 (FIG. 20B), or yield against the piston 1756 (FIG. 20C). Whether the resistive element 1750 will act as a rigid body or yield depends upon whether the resistance to movement of the piston 1756 is greater than the resistance to further compression of the resistive element 1750. Because the piston 1756 is coupled to the actuator arm 1704 via the piston arm 1760, the resistance to movement of the piston 1756 depends on the resistance to movement of the actuator arm 1704. Moving the actuator arm 1704 rotates the actuator body 1002, which in turn rotates the main winch shaft 1720 and the spool 1706 to further wind the cable 1620 onto the spool 1706 to incrementally increase the tension in the cable 1620. Therefore, the resistance to movement of the piston 1756 depends on the tension in the cable 1620.

More particularly, the piston arm 1760 projecting through the articulation aperture 1762 braces the piston 1756 against the actuator arm 1704. Therefore, the piston 1756 cannot move with the platform 1646 as the platform 1646 moves into the proximal position unless the actuator arm 1704 can move into the actuated position by rotating the actuator body 1702 to pivot the actuator 1640. Since the actuator 1640 is bound to the main winch shaft 1720 by the one-way actuator needle bearing 1718, the actuator 1640 can only pivot if the main winch shaft 1720 can rotate. Because the main winch shaft 1720 is fixed to the spool 1706, the main winch shaft 1720 can only rotate if the spool 1706 can also rotate. Since the cable 1620 is wound onto the spool 1706 and is under tension, the spool 1706 can only rotate if the existing tension in the cable 1620 can be overcome. Thus, the tension in the cable 1620 propagates back through the spool 1706, main winch shaft 1720 and actuator needle bearing 1718 to resist pivoting of the actuator 1640, thereby resisting movement of the actuator arm 1704 from the rest position to the actuated position and providing the resistance to movement of the piston 1756 by via the piston arm 1760 coupled to the piston 1756.

The tension in the cable 1620 tightens the panels 1616 against the residuum, and therefore the amount of tension in the cable 1620 corresponds to the tightness of the retention mechanism comprising the panels 1616. If the tension in the cable 1620 is below a threshold, when the end effector 1615 engages the surface, the winch assembly 1630 moves to the configuration in FIG. 20B, which rotates the spool 1706 to increase tension in the cable 1620. However, when the tension in the cable 1620 is at or above the threshold, when the end effector 1615 engages the surface the winch assembly 1630 moves to the configuration in FIG. 20C, which fails to rotate the spool 1706 and therefore will not increase the tension in the cable 1620. The threshold for the tension in the cable 1620 corresponds to the resistance of the resistive element 1750, which may be adjusted by tightening or loosening the cylinder setscrew 1754 so that the threshold is adjustable. Moreover, the resistance of the resistive element 1750 and the cushioning element 1746 need not be identical.

Reference is first made to FIG. 20B, which illustrates the scenario where the amount of force required to further compress the resistive element 1750 exceeds the resistance to movement of the piston 1756 resulting from the tension in the cable 1620 (i.e. tension in the cable 1620 is below the threshold). Therefore, the resistive element 1750 acts as a rigid body that pins the piston 1756 against the head 1758 of the cylinder barrel 1752 so that the piston 1756 cannot move along the cylinder barrel 1752 toward the cylinder setscrew 1754. Accordingly, movement of the platform 1646 into the proximal position will drive the piston arm 1760 into the actuator arm 1704 to move the actuator arm 1704 into the actuated position and rotate the actuator body 1702. Rotation of the actuator body 1702 rotates the main winch shaft 1720 and the spool 1706 to further wind the cable 1620 onto the spool 1706 to incrementally increase the tension in the cable 1620. Thus, where the tension in the cable 1620 is below the threshold, where there is movement of the platform 1646 toward the proximal position, the resistive element 1750 transmits this movement via the piston 1756 and piston arm 1760 to the actuator arm 1704 to pivot the actuator 1640 and thereby index the spool 1706. Accordingly, when the tightness of the retention mechanism comprising the panels 1616 and cable 1620 is below a desired threshold, each step transmits motion across the resistive elements 1750 to the actuator 1640 to cycle the actuator 1640. Each movement of the actuator arm 1704 into the actuated position rotates the actuator body 1702, which (via the actuator needle bearing 1718) rotates the main winch shaft 1720 and thereby rotates the spool 1706 to wind the cable 1620 onto the spool 1706 to incrementally increase the tension in the cable 1620, which further tightens the retention mechanism.

More particularly, movement of the actuator arm 1704 from a rest position (see FIG. 20A) to an actuated position (see FIG. 20B) will rotate (“rock”) the actuator body 1702 (clockwise in the Figures). Rotation of the actuator body 1702 is in the same rotational direction as the permitted direction of rotation of the actuator needle bearing 1718. Because the actuator needle bearing 1718 is friction fit within the actuator body 1702, the actuator needle bearing 1718 rotates along with the actuator body 1702. Rotation of the actuator needle bearing 1718 in the same rotational direction that the main winch shaft 1720 is permitted to rotate within the actuator needle bearing 1718 is equivalent to attempted rotation of the main winch shaft 1720 opposite to the permitted direction of rotation within the actuator needle bearing 1718. Therefore, the actuator needle bearing 1718 binds on the main winch shaft 1720 so that rotation of the actuator body 1702 drives rotation of the main winch shaft 1720 clockwise. Since the actuator needle bearing 1718 and the winch needle bearing 1714 on the main winch shaft 1720 have a common permitted direction of rotation, the main winch shaft 1720 can rotate within the winch needle bearing 1714 until the actuator arm 1704 reaches the actuated position (see FIG. 20B). Because the permitted direction of rotation is in a winding direction of the spool 1706, movement of the actuator arm 1704 from the rest position (see FIG. 20A) to the actuated position (see FIG. 20B) will incrementally rotate the main winch shaft 1720 and the spool 1706 coupled thereto in the winding direction of the spool 1706. This incrementally winds the cable 1620 (see FIGS. 18 and 19) further onto the spool 1706 and incrementally increases the tension in the cable 1620. The winch needle bearing 1714 resists rotation in the unwinding direction of the spool 1706 so that tension on the cable 1620 is maintained.

From the actuated position shown in FIG. 20B, the actuator arm 1704 can reciprocate back to the rest position shown in FIG. 20A, thereby rotating the actuator body 1702 back in the opposite rotational direction (anticlockwise in the Figures). This rotation of the actuator body 1702 and the actuator needle bearing 1718 therein is in the opposite rotational direction to the permitted direction of rotation of the actuator needle bearing 1718, which is equivalent to rotation of the main winch shaft 1720 in the permitted direction of rotation within the actuator needle bearing 1718. As a result, the actuator needle bearing 1718 and the actuator body 1702 can slip over the main winch shaft 1720 until the actuator arm 1704 returns to the rest position shown in FIG. 20A. Simultaneously, because rotation of the actuator body 1702 and the actuator needle bearing 1718 therein is in the opposite rotational direction to the permitted direction of rotation of the winch needle bearing 1714, the winch needle bearing 1714 inhibits the main winch shaft 1720 from rotating back with the actuator body 1702 and the actuator needle bearing 1718. Thus, each cycle of the actuator 1640 from the rest position (FIG. 20A) to the actuated position (FIG. 20B) and back to the rest position (FIG. 20A) incrementally rotates (indexes) the main winch shaft 1720. Since rotation of the main winch shaft 1720 will result in rotation of the spool 1706 by way of interengagement of the crossbar arms 1722 with the locking channels 1740, each cycle of the actuator 1640 from the rest position (FIG. 20A) to the actuated position (FIG. 20B) and back to the rest position (FIG. 20A) incrementally rotates (indexes) the spool 1706. Thus, each cycle of the actuator 1640 indexes the spool 1706 to wind the cable 1620 onto the spool 1706 to incrementally increase the tension in the cable 1620. Accordingly, the actuator 1640 is configured to reciprocally cycle between a rest position and an actuated position and to act through the mechanical linkage comprising the cables 1620, via the winch assembly 1630, to incrementally tighten the retention mechanism comprising the panels 1616 to incrementally tighten the retention mechanism against the residuum on each movement of the actuator 1640 into the actuated position and leave the retention mechanism further incrementally tightened upon each return of the actuator 1640 to the rest position.

Reference is now made to FIG. 20C, which illustrates the scenario where the amount of force required to further compress the resistive element 1750 is less than the resistance to movement of the piston 1756 resulting from the tension in the cable 1620 (FIGS. 18 and 19) transmitted back to the actuator arm 1704. More force is required to overcome the tension in the cable 1620 and move the actuator arm 1704 from the rest position to the actuated position than is required to further compress the resistive element 1750. Therefore, instead of movement of the platform 1646 being transmitted through the resistive element 1750 to move the piston 1756 and pivot the actuator 1640, the resistive element 1750 acts like a spring rather than a rigid body. The resistive element 1750 is compressed between the cylinder setscrew 1754 and the piston 1756 as the platform 1646 moves, with the piston 1756 sliding along the inside of the cylinder barrel 1752 instead of being pinned against the head 1758 thereof. The resistance of the resistive element 1750 cannot overcome the tension in the cable 1620, so the resistive element 1750 yields (e.g. the spring is compressed). Accordingly, instead of the piston arm 1760 moving the actuator arm 1704 toward the actuated position, the actuator arm 1704 is maintained in position by the tension in the cable 1620 and the piston 1756 is pushed into the cylinder barrel 1752 by the piston arm 1760 and the piston 1756 compresses the resistive element 1750 against the cylinder setscrew 1754. Thus, when the tightness of the retention mechanism comprising the panels 1616 has reached the threshold, on each further step the resistive elements 1750 yield to absorb the motion of the platform 1646, rather than transmitting the motion of the platform 1646 to the actuator 1640. When the resistive elements 1750 yield instead of transmitting motion, the actuator 1640 will fail to cycle on each further step, thereby inhibiting further tightening of the retention mechanism beyond the threshold.

When the user removes the weight from the end effector 1615, the cushioning element 1746 returns the platform 1646 from the proximal position (FIGS. 20B and 20C) to the distal position (FIG. 20A).

Accordingly, as has been shown with respect to FIGS. 13A through 13C, reciprocal movement of the platform 1646 into the proximal position and back to the distal position cycles the actuator 1640 only where the resistance to compression of the resistive element 1750 exceeds the resistance to movement of the actuator 1640 resulting from the tension in the cable 1620 so that the resistive element 1750 transmits the movement of the platform 1646 to the actuator 1640 instead of yielding to the movement of the platform 1646.

Thus, in the illustrated embodiment, the resistive element 1750, actuator 1640 and actuator needle bearing 1718 provide a mechanical interface to transmit movement of the end effector 1615 of the lower limb prosthesis toward the residuum resulting from a step to a tensioner comprising the main winch shaft 1720, release shaft 1724, crossbar arms 1722, and spool 1706, with resistance of the mechanical interface provided by the resistive element 1750 (e.g. a spring).

The winch assembly 1630 shown in FIGS. 17 through 22B may be provided without any receptacle body 1604 or end effector 1615, for example as a kit of parts, with or without the lamination plate 1674, for use by a trained prosthetist in constructing a socket 1600.

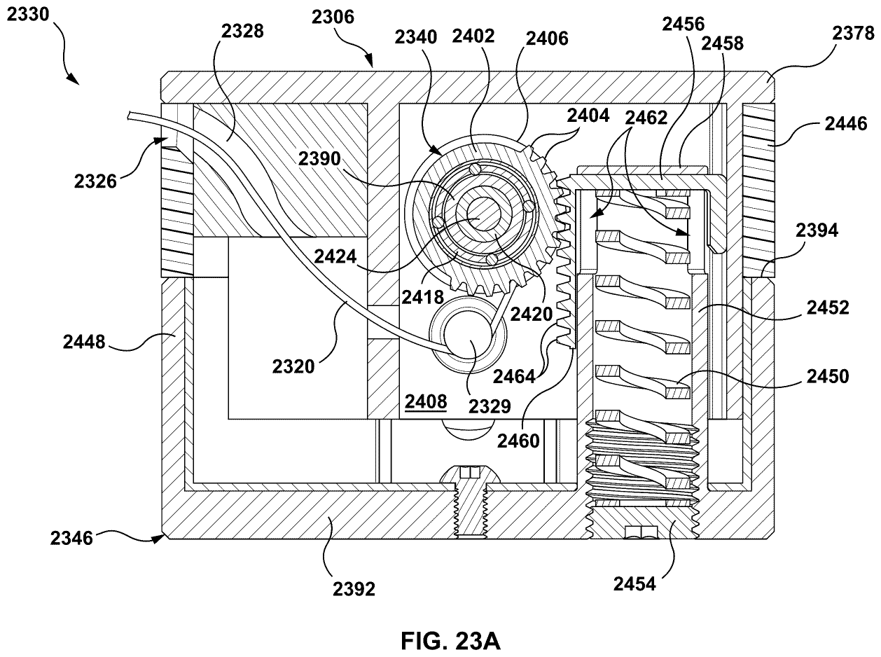

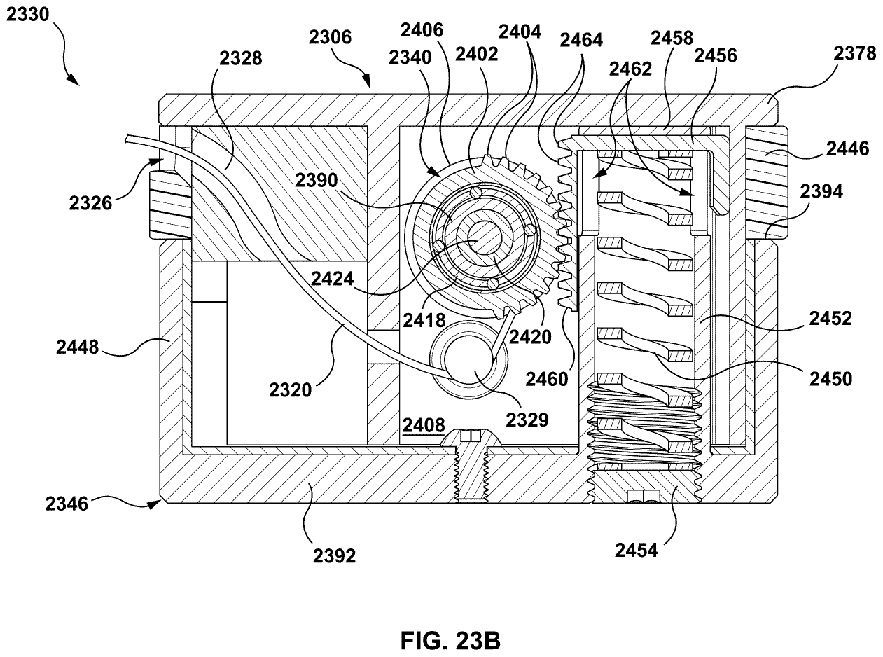

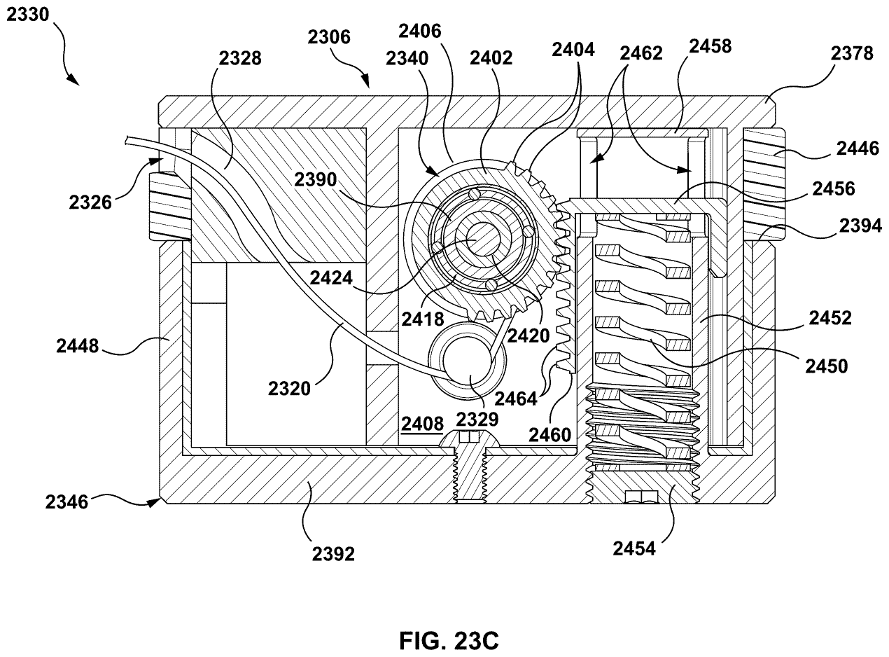

Reference is now made to FIGS. 23A through 23C, which show another illustrative winch assembly 2330, which may be used as part of a socket adjustment mechanism for a lower limb prosthesis. The structure of the winch assembly 2330 shown in FIGS. 23A through 23C is similar to that of the winch assembly 1630 described above, with like reference numerals generally denoting like features except with the prefix “23” and “24” rather than “16” and “17”, respectively. The structure of the winch assembly 2330 shown in FIGS. 23A through 23C differs from that of the winch assembly 1630 described above in two primary ways. First, the winch assembly 2330 uses a rack and pinion arrangement as described further below, and second, the winch assembly 2330 uses an annular cushioning element 2446, again as described further below.

An actuator enclosure 2306 is connectable to a residuum receptacle, and functions as a winch body that carries the components of the winch assembly 2330. A generally circular lamination plate (similar to the lamination plate 1674 described above) can be integrated into a lower limb prosthesis in known manner, and then the actuator enclosure 2306 can be coupled to the lamination plate. For example, the actuator enclosure 2306 can be mounted to a standard lamination plate through the use of an industry standard 4-hole adapter pattern, or a retaining collar can be used with a threaded ring, or inwardly facing retaining screws, or any other suitable mechanism may be used to releasably couple the actuator enclosure 2306 to the residuum receptacle.

In the illustrated embodiment, the winch assembly 2330 comprises a single actuator 2340, which is disposed within the actuator enclosure 2306. The actuator 2340 comprises a pinion 2340 having a tubular cylindrical pinion body 2402 and a plurality of circumferentially spaced outwardly extending pinion teeth 2404. In the illustrated embodiment the pinion teeth 2404 extend only partially around the circumference of the pinion 2340 although in other embodiments the pinion teeth may extend around the complete circumference of the pinion. The pinion teeth 2404 can be conceptualized as individual actuator arms that interact with a rack 2460 (described further below) and act as levers enabling the pinion 2340 to rock back and forth about an axis extending through the pinion body 2402 as the rack 2460 moves linearly. Thus, the pinion 2340 can reciprocally cycle between a rest position and an actuated position.

The pinion 2340 is coupled to a spool 2406 in similar manner as was described above in respect of the actuator 1640 and spool 1706 shown in FIGS. 17 to 22B, the details of which are not shown or repeated for the sake of brevity, and the pinion 2340 is connectable to a residuum retention mechanism via the spool 2306, onto which a cable 2320 may be wound and from which the cable 2320 may be unwound to respectively tighten and loosen the retention mechanism as already described above. The pinion 2340 and the spool are disposed in a winch cavity 2408 formed within the actuator enclosure 2306, similarly to the arrangement of the actuator 1640 and the spool 1706 within the winch cavity 1708 as described above in the context of FIGS. 17 to 22B, with a similar bearing arrangement comprising opposed first and second bearing apertures, a winch roller bearing (not shown) and a one-way winch needle bearing (not shown) Analogously to the embodiment shown in FIGS. 17 to 22B, the pinion 2340 is releasably coupled to the spool 2406 by way of a one-way pinion needle bearing 2418 friction fit into the opening of the pinion body 2402, a hollow main winch shaft 2420 at least partially surrounded by a friction sleeve 2390, and a release shaft 2424 disposed concentrically within the lumen of the hollow main winch shaft 2420. The main winch shaft 2420, with the friction sleeve 2390, passes through the winch roller bearing, pinion needle bearing 2418, and winch needle bearing, the latter two being arranged to have a common permitted direction of rotation. The main winch shaft 2420 terminates with a manual tensioning knob (not shown) disposed outside of the actuator enclosure 2306. The release shaft 2424 can engage and disengage the main winch shaft 2420 from the spool 2406 in the same manner as described above in the context of FIGS. 21A and 21B, and these details are not repeated.

Motion from steps taken with the prosthesis is transmitted to the pinion 2340 by a movable platform 2346. In the illustrated embodiment the movable platform 2346 is carried by the actuator enclosure 2306. The movable platform 2346 comprises a generally circular platform base 2392 that supports a hollow cylinder barrel 2452 and is circumvallated by an annular cushioning interface wall 2448. The movable platform 2346 is coupled to the actuator enclosure 2306 at least in part by an annular cushioning ring 2446 formed from a resilient material, for example urethane, and which is interposed between an outwardly extending annular enclosure flange 2378 at the proximal end of the actuator enclosure 2306 and the proximal surface 2394 of the cushioning interface wall 2448. In one embodiment, a suitable adhesive may be used to adhere the cushioning ring 2446 to the annular enclosure flange 2378 and to the proximal surface 2394 of the cushioning interface wall 2448. In another embodiment, retaining screws may be used; these may also be used to enforce travel limits on the platform 2346 relative to the actuator enclosure 2306. Thus, the annular cushioning ring 2446 acts between the actuator enclosure 2306 and the movable platform 2346. This allows the movable platform 2346 to reciprocate between a proximal position (closer to the proximal end of the actuator enclosure 2306) and a distal position (further from the proximal end of the actuator enclosure 2306).

A port 2326 is formed through the cushioning ring 2446 and leads through the port 2326 into and through a passageway 2328 and into the winch cavity 2408. The port 2326 may be reinforced, for example by a grommet, to prevent the port 2326 from collapsing during compression of the cushioning ring 2446 as described below and to inhibit friction damage to the cushioning ring 2446. The cable 2320 enters the actuator enclosure 2306 through the port 2326 and is guided by the passageway 2328 to an idler pulley 2329, which redirects the cable 2320 to the spool 2406 to which ends of the cable 2320 are fixed.

A resistive element 2450 is disposed in the cylinder barrel 2452, which is internally threaded at its distal end to receive a cylinder setscrew 2454 that serves as a cylinder cap. While the resistive element 2450 is shown as a compression spring, this is merely illustrative and not limiting. The cylinder barrel 2452 includes diametrically opposed axially extending piston slots 2462 adjacent the head 2458 of the cylinder barrel 2452. A piston 2456 extends through the cylinder barrel 2452 and is trapped in the piston slots 2462 so as to be axially movable along the cylinder barrel 2452 toward and away from the head 2458 of the cylinder barrel 2452 within the travel limits defined by the piston slots 2462.

The piston 2456 fixedly carries a rack 2460 positioned so that the rack teeth 2464 mesh with the pinion teeth 2404 on the pinion 2340, forming a rack-and-pinion arrangement. Axial movement of the piston 2456 will cause the rack 2460 to also move axially, and thereby rotate the pinion 2340. Thus, movement of the piston 2456 and with it the rack 2460 can drive reciprocal pivotal movement of the pinion 2340 between a rest position (FIG. 23A) and an actuated position (FIG. 23B).

Each time a user takes a step with the lower limb prosthesis, when the user puts weight on the end effector (not shown) coupled to the movable platform 2346, the weight will compress the cushioning ring 2446 and move the platform 2346 from the distal position into the proximal position. Movement of the platform 2346 is transmitted to the resistive element 2450 trapped between the platform base 2392 (in particular the cylinder setscrew 2454) and the piston 2456. The resistive element 2450 is mechanically coupled to the pinion 2340 via the piston 2456 and the rack 2460.

FIG. 23A shows the pinion 2340 in the rest position with the platform 2346 in the distal position; FIG. 23B shows the pinion 2340 in the actuated position with the platform 2346 in the proximal position, and FIG. 23C shows the pinion 2340 in the rest position with the platform 2346 in the proximal position. Because in the illustrated embodiment the pinion teeth 2404 extend only partially around the circumference of the pinion 2340, the rest position of the pinion 2340 and the actuated position of the pinion can be easily distinguished in the Figures. As noted above, in other embodiments the pinion teeth may extend around the entire circumference of the pinion.

FIG. 23A shows the configuration of the winch assembly 2330 when a user is not applying weight to the end effector; application of weight to the end effector moves the winch assembly 2330 either to the configuration in FIG. 23B, or to the configuration in FIG. 23C, depending on the tension in the cable 2320. Application of the user's weight to the end effector compresses the cushioning ring 2446 and moves the platform 2346 from the distal position (FIG. 23A) into the proximal position (FIGS. 23B and 23C). Because the resistive element 2450 is trapped in the cylinder barrel 2452 between the cylinder setscrew 2454 and the piston 2456, movement of the platform 2346 applies force to the resistive element 2450 via the cylinder setscrew 2454. As the platform 2346 moves from the distal position (FIG. 23A) into the proximal position (FIGS. 23B and 23C), the resistive element 2450 will either act as a rigid body bracing the piston 2456 against the head 2458 of the cylinder barrel 2452 (FIG. 23B), or yield against the piston 2456 (FIG. 23C) depending on whether the resistance to movement of the piston 2456 is greater than the resistance to further compression of the resistive element 2450. Because the piston 2456 is coupled to the pinion 2340 by intermeshing of the rack teeth 2464 with the pinion teeth 2404, the resistance to movement of the piston 2456 depends on the resistance to rotation of the pinion 2340. Moving the pinion 2340 rotates the main winch shaft 2420 and the spool 2406 to further wind the cable 2320 onto the spool 2406 to incrementally increase the tension in the cable 2320. Accordingly, the resistance to movement of the piston 2456 depends on the tension in the cable 2320.

More particularly, since the piston 2456 fixedly carries the rack 2460, the piston 2456 cannot move with the platform 2346 as the platform 2346 moves into the proximal position unless the rack 2460 can also move with the movable platform 2346. But, because the rack teeth 2464 intermesh with the pinion teeth 2404, the rack 2460 cannot move axially unless the pinion 2340 rotates. Since the pinion 2340 is bound to the main winch shaft 2420 by the one-way pinion needle bearing 2418, the pinion 2340 can only rotate if the main winch shaft 2420 can also rotate, and the main winch shaft 2420 is fixed to the spool 2406 and can only rotate if the spool 2406 can also rotate. Because the cable 2320 is wound onto the spool 2406 under tension, the spool 2406 can only rotate if the existing tension in the cable 2320 can be overcome. Accordingly, the tension in the cable 2320 propagates back through the spool 2406, main winch shaft 2420 and pinion needle bearing 2418 to resist rotation of the pinion 2340, thereby resisting movement of the rack 2460 and hence also providing resistance to movement of the piston 2456 that fixedly carries the rack 2460.

The tension in the cable 2320 tightens the panels against the residuum, and therefore the amount of tension in the cable 2320 corresponds to the tightness of the retention mechanism comprising the panels. If the tension in the cable 2320 is below a threshold, when the end effector engages the surface, the winch assembly 2330 moves to the configuration in FIG. 23B, which rotates the spool 2406 to increase tension in the cable 2320. However, when the tension in the cables 2320 is at or above the threshold, when the end effector engages the surface the winch assembly 2330 moves to the configuration in FIG. 23C, which fails to rotate the spool 2306 and therefore will not increase the tension in the cable 2320. The threshold for the tension in the cable 2320 corresponds to the resistance of the resistive element 2450, which is adjustable via the cylinder setscrew 2454 to provide for an adjustable threshold. The resistance of the resistive element 2450 and the cushioning ring 2446 need not be identical.

Reference is first made to FIG. 23B, which illustrates the scenario where the amount of force required to further compress the resistive element 2450 exceeds the resistance to movement of the piston 2456 resulting from the tension in the cable 2320 via interaction between the rack teeth 2464 and the pinion teeth 2404 (i.e. tension in the cable 2320 is below the threshold). In this scenario, the resistive element 2450 acts as a rigid body that pins the piston 2456 against the head 2458 of the cylinder barrel 2452 so that the piston 2456 is unable to move along the piston slots 2462 in the cylinder barrel 2452 toward the cylinder setscrew 2454. Thus, when the platform 2346 moves into the proximal position, the piston 2456 and the rack 2460 (fixedly carried by the piston 2466) move with the platform 2346 and this movement of the rack 2460 will rotate the pinion 2340 from the rest position into the actuated position. Rotation of the pinion 2340 rotates the main winch shaft 2420 and the spool 2406 to further wind the cable 2320 onto the spool 2406 to incrementally increase the tension in the cable 2320. Accordingly, when the tension in the cable 2320 is below the threshold, movement of the platform 2346 toward the proximal position is transmitted by the resistive element 2450, the piston 2456, and the rack 2460, to the pinion 2340 to rotate the pinion 2340 and thereby index the spool 2406.

Thus, where the resistance to compression of the resistive element 2450 exceeds the resistance to movement from the tension in the cable 2320, the resistive element 2450 transmits the movement of the platform 2346 into the proximal position to the rack 2460 engaged with the pinion 2340 to rotate the pinion and thereby index the spool 2406.

In more detail, with reference to FIGS. 23A and 23B, movement of the pinion 2340 from a rest position (see FIG. 23A) to an actuated position (see FIG. 23B) will rotate (“rock”) the pinion body 2402 (anticlockwise in FIGS. 23A and 23B). The pinion body 2402 rotates in the permitted direction of rotation of the pinion needle bearing 2418, and the pinion needle bearing 2418 (friction fit within the pinion body 2402) rotates along with the pinion body 2402. The rotation of the pinion needle bearing 2418 is in the same rotational direction that the main winch shaft 2420 is permitted to rotate within the pinion needle bearing 2418. This is equivalent to attempted rotation of the main winch shaft 2420 opposite to the permitted direction of rotation within the pinion needle bearing 2418, so the pinion needle bearing 2418 binds on the main winch shaft 2420 whereby the rotation of the pinion body 2402 drives rotation of the main winch shaft 2420 (anticlockwise in FIGS. 23A and 23B). Since the pinion needle bearing 2418 and the winch needle bearing have a common permitted direction of rotation, the main winch shaft 2420 can rotate within the winch needle bearing until the pinion 2340 reaches the actuated position (see FIG. 23B). Because the permitted direction of rotation is in a winding direction of the spool 2406, movement of the pinion 2340 from the rest position (see FIG. 23A) to the actuated position (see FIG. 23B) will incrementally rotate the main winch shaft 2420 and the spool 2406 coupled thereto in the winding direction of the spool 2406. This incrementally winds the cable 2320 further onto the spool 2406 and incrementally increases the tension in the cable 2320. The winch needle bearing resists rotation in the unwinding direction of the spool 2306 so that tension on the cable 2320 is maintained.

From the actuated position shown in FIG. 23B, the pinion 2340 can rotate back to the position shown in FIG. 23A, thereby rotating the pinion body 2402 and the pinion needle bearing 2418 back in the opposite rotational direction to the permitted direction of rotation of the pinion needle bearing 2418 (clockwise in FIGS. 23A and 23B). This is equivalent to rotation of the main winch shaft 2420 in the permitted direction of rotation within the pinion needle bearing 2418, so the pinion needle bearing 2418 and the pinion body 2402 can slip over the main winch shaft 2420 until the pinion 2340 returns to the rest position shown in FIG. 23A. Simultaneously, because rotation of the pinion body 2402 and the pinion needle bearing 2418 therein is in the opposite rotational direction to the permitted direction of rotation of the winch needle bearing, the winch needle bearing inhibits the main winch shaft 2420 from rotating back with the pinion body 2402 and the actuator needle bearing 2418. Each cycle of the pinion 2340 from the rest position (FIG. 23A) to the actuated position (FIG. 23B) and back to the rest position (FIG. 23A) incrementally rotates (indexes) the main winch shaft 2420. Since rotation of the main winch shaft 2420 will result in rotation of the spool 2406, each cycle of the pinion 2340 from the rest position (FIG. 23A) to the actuated position (FIG. 23B) and back to the rest position incrementally rotates (indexes) the spool 2406. Thus, each cycle of the pinion 2340, which is coupled to the spool 2406, indexes the spool 2306 to wind the cable 2320 onto the spool 2306 to incrementally increase the tension in the cable 2320. Accordingly, the pinion 2340 is configured to reciprocally cycle between a rest position and an actuated position and to act through the mechanical linkage comprising the cables 2320, via the winch assembly 2330, to incrementally tighten the retention mechanism against the residuum on each movement of the pinion 2340 into the actuated position and leave the retention mechanism further incrementally tightened upon each return of the pinion 2340 to the rest position.

Thus, when the tightness of the retention mechanism comprising the panels and cables 2320 is below a desired threshold, each step transmits motion across the resistive elements 2450 to the pinions 2340 to cycle the pinion 2340. Each movement of the pinion 2340 into the actuated position rotates the pinion body 2402, which (via the actuator needle bearing 2418) rotates the main winch shaft 2420 and thereby rotates the spool 2406 to wind the cable 2320 onto the spool 2406 to incrementally increase the tension in the cable 2320, which further tightens the retention mechanism.

Reference is now made to FIG. 23C, which illustrates the scenario where the amount of force required to further compress the resistive element 2450 is less than the resistance to movement of the piston 2456 resulting from the tension in the cable 2320 transmitted back to the pinion 2340. More force is required to overcome the tension in the cable 2320 and move the pinion 2340 from the rest position to the actuated position than is required to further compress the resistive element 2450. Therefore, instead of movement of the platform 2346 being transmitted through the resistive element 2450 to move the piston 2456 and thereby move the rack 2460 to pivot the pinion 2340, the resistive element 2450 acts like a spring rather than a rigid body. The resistive element 2450 is compressed between the cylinder setscrew 2454 and the piston 2456 as the platform 2346 moves, with the piston 2456 moving along the piston slots 2462 in the cylinder barrel 2452 toward the cylinder setscrew 2454 instead of being pinned against the head 2458 of the cylinder barrel 2452. The resistance of the resistive element 2450 is unable to overcome the tension in the cable 2320, so the resistive element 2450 yields (e.g. the spring is compressed).

Accordingly, instead of the rack 2460 moving the pinion 2340 toward the actuated position, the rack 2460 is maintained in position by the tension in the cable 2320 acting through the pinion 2340. Since the rack 2460 is fixedly carried by the piston 2456, the piston 2456 is pushed into the cylinder barrel 2452 by the rack 2460 and the piston 2456 compresses the resistive element 2450 against the cylinder setscrew 2454 as the platform moves into the proximal position. Thus, when the tightness of the retention mechanism has reached the threshold, on each further step the resistive element 2450 yields to absorb the motion of the platform 2346, rather than transmitting the motion of the platform 2346 to through the piston 2456 and rack 2460 to the pinion 2340. When the resistive elements 2450 yield instead of transmitting motion, the pinion 2340 will fail to cycle on each further step, thereby inhibiting further tightening of the retention mechanism beyond the threshold.

When the user removes the weight from the end effector, the cushioning ring 2446 returns the platform 2346 from the proximal position (FIGS. 23B and 23C) to the distal position (FIG. 23A).

Accordingly, as has been shown with respect to FIGS. 23A through 23C, reciprocal movement of the platform 2346 into the proximal position and back to the distal position will only cycle the actuator 2340 if the resistance to compression of the resistive element 2450 exceeds the resistance to movement of the pinion 2340 resulting from the tension in the cable 2320 so that the resistive element 2450 transmits the movement of the platform 2346 to the pinion 2340 instead of yielding to the movement of the platform 2346.

Thus, in the illustrated embodiment, the resistive element 2450, rack 2460, pinion 2340 and pinion needle bearing 2418 provide a mechanical interface to transmit movement of the end effector of the lower limb prosthesis toward the residuum resulting from a step to a tensioner comprising the main winch shaft 2420, release shaft 2424, crossbar arms, and spool 2406, with resistance of the mechanical interface provided by the resistive element 2450 (e.g. a spring).

In the illustrative implementations described above, each cycle of the actuator 140, 1640, 2340 incrementally winds a cable 120, 1620, 2320 around a spool 1006, 1706, 2406 to increase tension in the cable 120, 1620, 2320, with the actuator 140, 1640, 2340 and the spool 1006, 1706, 2406 both being carried on a single common main winch shaft 1020, 1720, 2420. However, this is merely one illustrative, non-limiting illustration and other arrangements are contemplated. In other embodiments, the actuator may be carried on an actuator shaft and the spool may be carried on a spool shaft that is different from the actuator shaft, with the actuator shaft and the spool shaft being coupled to one another (e.g. by suitable intermediate gearing or otherwise) so that rotation of the actuator shaft causes rotation of the spool shaft. One example of The intermediate gearing may be configured to provide a mechanical advantage; without limitation a mechanical advantage of about 6 is currently preferred. For example, the winch assembly 2330 shown in FIGS. 23A to 23C may be modified by omitting the idler pulley 2329 and mounting the spool on a spool shaft rotating about the same axis as the idler pulley 2329. The pinion, pinion needle bearing and friction sleeve would then be mounted on an actuator shaft replacing main winch shaft 2420, with intermediate gearing interposed between the actuator shaft and the spool shaft so that rotation of the actuator shaft will drive rotation of the spool shaft. The spool shaft may include a release mechanism similar to that described above in the context of the main winch shaft 2420. The foregoing is merely an illustrative, non-limiting example of a multi-shaft embodiment. For example, in other embodiments the actuator shaft and the spool shaft may be non-parallel.

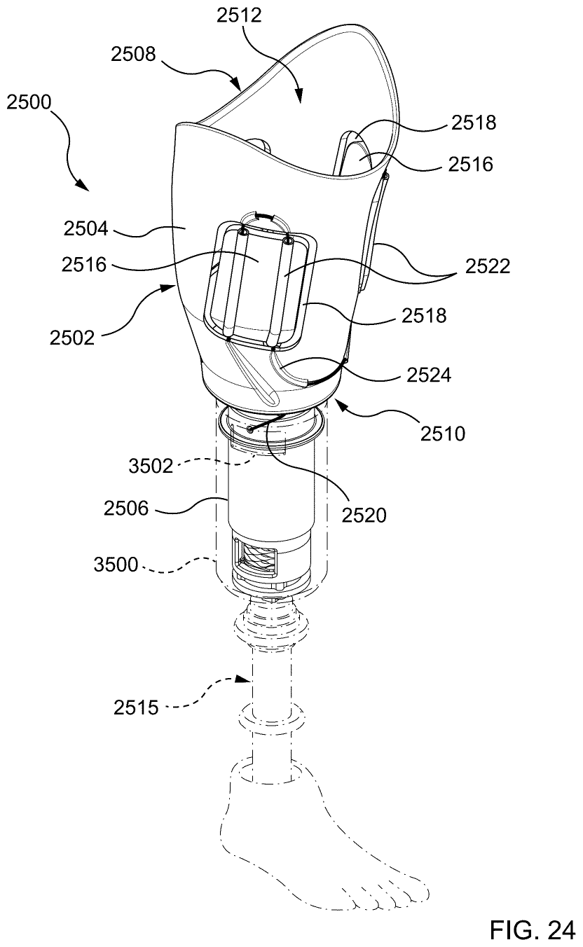

Reference is now made to FIG. 24, which shows yet another non-limiting illustrative embodiment of a self-adjusting socket 2500 for a lower limb prosthesis. While the illustrative socket 2500 is for a transtibial prosthesis, it can be adapted for use with a socket for a transfemoral amputee by one of ordinary skill in the art, now informed by the present disclosure. In the illustrated embodiment, the self-adjusting socket 2500 comprises a housing 2502, which in turn comprises a receptacle body 2504 and an actuator enclosure 2506 carried by the receptacle body 2504. The receptacle body 2504 is similar to that described above, being generally hollow with an open end 2508 and a support end 2510 opposite the open end 2508. The interior volume of the receptacle body 2504 forms a residuum receptacle 2512 adapted to receive the residuum of an amputee via the open end 2508. Thus, the housing 2502, specifically the receptacle body 2504 thereof, comprises a residuum receptacle 2512. An end effector 2515 is coupled to the socket 2500 via the actuator enclosure 2506. The actuator enclosure 2506 encloses additional components of a socket adjustment mechanism of which the actuator enclosure 2506 also forms a part.

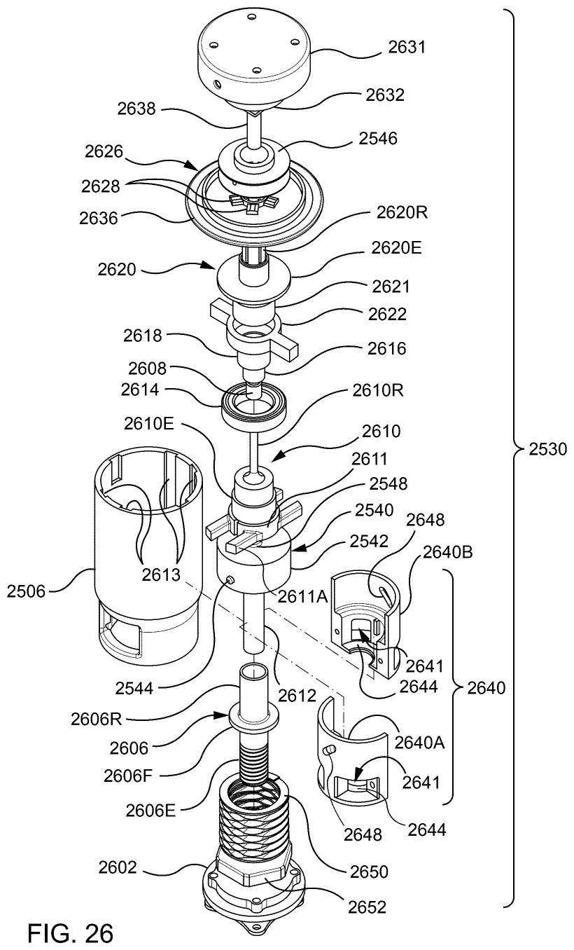

The housing 2502 carries a retention mechanism configured for retaining a residuum within the residuum receptacle 2512. In the illustrated embodiment, the retention mechanism comprises three panels 2516 arranged circumferentially about the residuum receptacle 2512 and disposed in respective openings 2518 in the receptacle body 2504. The panels 2516 may include interiorly facing pads for cushioning. The panels 2516 are movable so as to be inwardly and outwardly displaceable relative to the receptacle body 2504 of the housing 2502. This is merely a non-limiting illustrative embodiment; more or fewer panels may be present, or an alternate retention mechanism may be used. A single looped cable 2520 runs through tubular cable guides 2522 formed on the panels 2516 and through covered cable guides 2524 on the outer surface of the receptacle body 2504, with the ends of the cable 2520 being coupled to a winch assembly 2530 (see FIG. 26) that includes an actuator 2540, as described further below. The cable 2520 is thus coupled to the panels 2516. The actuator 2540 (see FIG. 26) is disposed within an actuator enclosure 2506, which is coupled to the residuum receptacle 2512 via the housing 2502, and the actuator 2540 is coupled to the retention mechanism through a mechanical linkage that includes the cable 2520 as well as other components of the winch assembly 2530 as described further below. The tightness of the retention mechanism can be adjusted by increasing and decreasing the tension in the cable 2520, in a manner similar to that described above, by way of the winch assembly 2530. As will be described below in greater detail, the actuator 2540 is configured to act through the mechanical linkage to incrementally move the panels 2516 inwardly to tighten the panels 2516 against the residuum on each cycle of the actuator 2540. More particularly, in the illustrated embodiment the actuator 2540 is configured to incrementally increase tension in the cable 2520 on each cycle of the actuator 2540, and incrementally increasing the tension on the cable 2520 moves the panels 2516 inwardly relative to the residuum receptacle 2512.

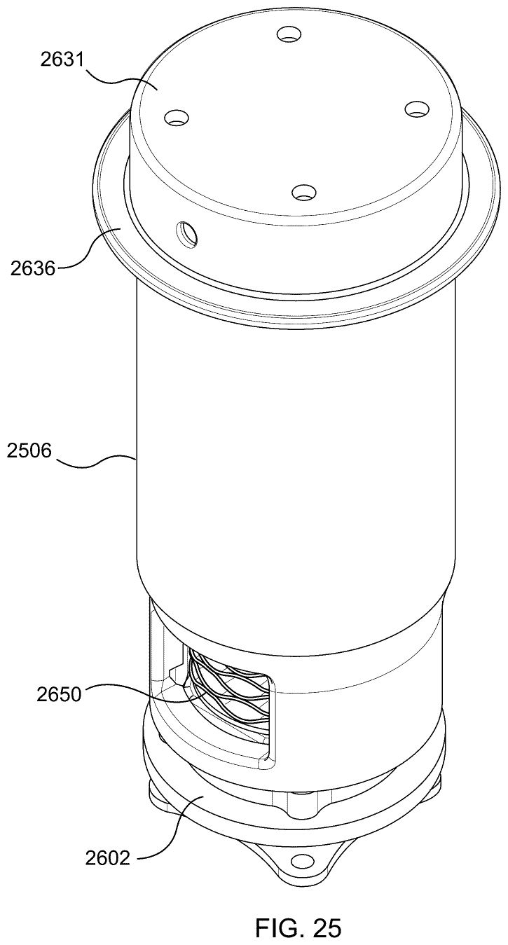

Reference is now made to FIG. 25, which shows the actuator enclosure 2506 in more detail. The actuator enclosure 2506 is generally cylindrical, with an inwardly necked effector end, and serves as a winch body for the winch assembly 2530, as shown in the exploded view in FIG. 26. The actuator enclosure 2506 carries a movable platform 2602 as part of the winch assembly 2530. A lamination adaptor 2631 is connectable to the receptacle body 2504 and is fixedly carried by the actuator enclosure 2506.

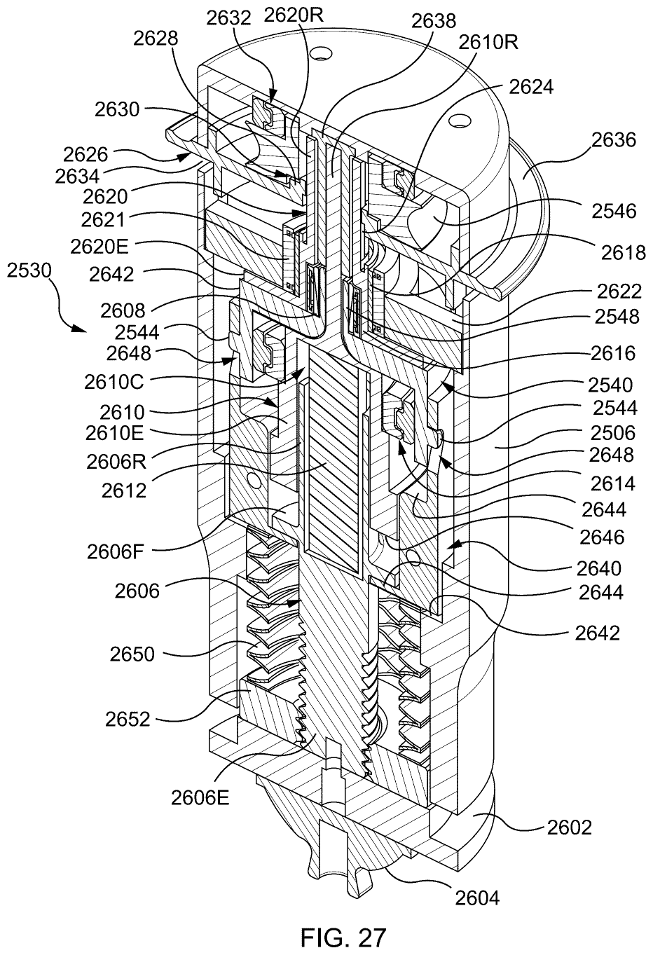

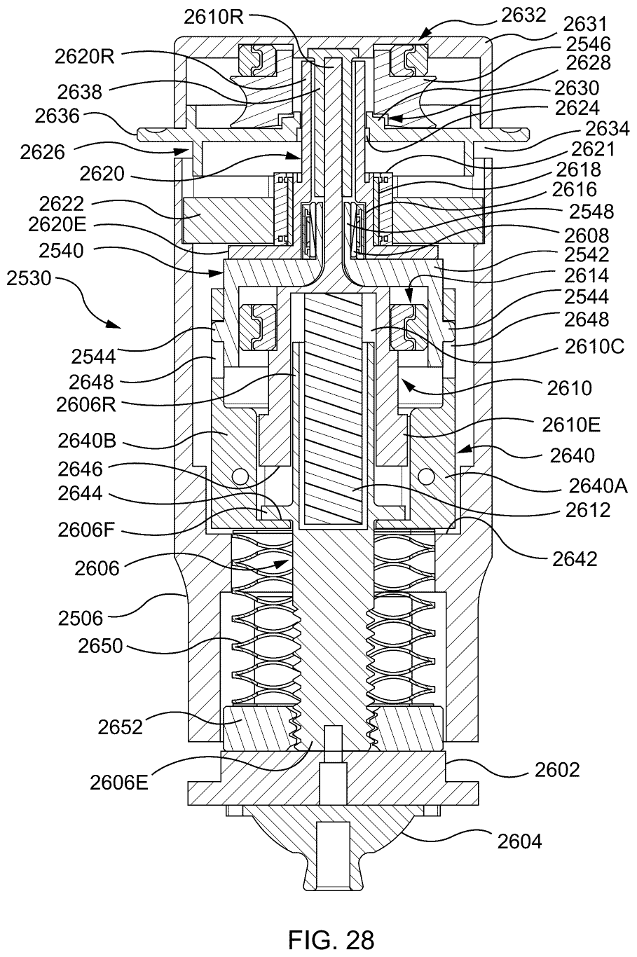

Referring now to FIGS. 26 through 28, a pyramid adaptor 2604 depends from the movable platform 2602 for connecting an end effector such as the end effector 2515 shown in FIG. 24. The pyramid adaptor 2604 is merely illustrative and not limiting; other suitable adaptors may be used. The movable platform 2602 is fixed to the effector end 2606E of a flanged movable shaft 2606, the effector end 2606E of the movable shaft 2606 being the end closest to the pyramid adaptor 2604, which can connect to an end effector. The movable shaft 2606 is movable axially within the actuator enclosure 2506. Thus, the movable platform 2602 and the movable shaft 2606 move in unison, and the movable platform is reciprocally movable relative to the actuator enclosure 2506 between a distal position (further from the lamination adaptor 2631, described further below) and a proximal position (closer to the lamination adaptor 2631). An annular flange 2606F divides the effector end 2606E of the movable shaft 2606 from a receptacle end 2606R of the movable shaft 2606. The receptacle end 2606R of the movable shaft 2606 is the end of the movable shaft 2606 that is distal from the movable platform 2602 and closer to the lamination adaptor 2631 that connects to the receptacle body 2504. The receptacle end 2606R of the movable shaft 2606 is hollow and is slidably received within a cylindrical axial cavity 2610C in the effector end 2610E of a fixed shaft 2610, that is, the end of the fixed shaft 2610 closest to the pyramid adaptor 2604. The fixed shaft 2610 is axially fixed relative to the actuator enclosure 2506, with the receptacle end 2610R forming a spindle and the effector end 2610E having a stepped campanulate outer surface. In the illustrated embodiment, the fixed shaft 2610 is fixed to the actuator enclosure 2506 by way of a brace 2611 whose arms 2611A are received in corresponding brace slots 2613 in the inner surface of the actuator enclosure 2506 (FIG. 26). A cushioning element 2612 is received within the hollow receptacle end 2606R of the movable shaft 2606, extending beyond the hollow receptacle end 2606R of the movable shaft 2606 to engage with the base of the axial cavity 2610C in the effector end 2610E of the fixed shaft 2610. Thus, the cushioning element 2612 is interposed between the movable shaft 2606 and the fixed shaft 2610. The cushioning element 2612 may be, for example, a urethane rod.

In the illustrated embodiment, the winch assembly 2530 comprises a single actuator 2540 having a tubular cylindrical actuator body 2542 and two diametrically opposed outwardly extending actuator arms 2544 that act as levers to pivot the actuator 2540. The actuator 2540 is a rocker that can rock back and forth about a central axis extending through the actuator body 2542; the actuator body 2542 is coaxial with the fixed shaft 2610 and rotates about the fixed shaft 2610. A two-way roller bearing 2614 is interposed between the actuator body 2542 and the fixed shaft 2610. In the illustrated embodiment, the actuator arms 2544 are cam followers, as will be described further below. This is a non-limiting illustrative embodiment of an actuator, and other types of actuators are also contemplated.

The actuator 2540 is coupled to a spool 2546 onto which the cable 2520 may be wound and from which the cable 2520 may be unwound to respectively tighten and loosen the retention mechanism comprising the panels 2516. In the illustrated embodiment the actuator 2540 further comprises a hollow axial axle 2548 extending toward the receptacle body 2504. The hollow axial axle 2548 is friction fit into a sleeve bearing 2608 that is rotatably received within a one-way needle roller bearing 2616 (inner one-way bearing). The one-way needle roller bearing 2616 is friction fit into a bushing 2618, which is in turn friction fit into a flanged effector end 2620E of a spool connector 2620. The spool connector 2620 is arranged coaxially with the actuator body 2542, fixed shaft 2610 and movable shaft 2606. At the effector end 2620E thereof, the spool connector 2620 is rotatably received within a one-way clutch bearing 2621 (outer one-way bearing). The one-way clutch bearing 2621 is friction fit into a bearing housing 2622 that is fixed to the actuator enclosure 2506. The receptacle end 2620R of the spool connector 2620 has a cruciate cross-section, and is slidably received within a correspondingly shaped central aperture 2624 of a spool lock member 2626 so that the spool connector 2620 and the spool lock member 2626 rotate in unison. The spool lock member 2626 has a plurality of locking projections 2628 extending toward the lamination adaptor 2631 that can connect to the receptacle body 2504. The locking projections 2628 are releasably received in correspondingly positioned locking cavities 2630 in the spool 2546. When the locking projections 2628 are received in the locking cavities 2630, the spool 2546 rotates in unison with the spool lock member 2626 and therefore the spool 2546 also rotates in unison with the spool connector 2620. The spool 2546 is rotatably coupled to the lamination adaptor 2631 by way of a two-way roller bearing 2632.

At least one opening 2634 is provided between the lamination adaptor 2631 and the actuator enclosure 2506 to accommodate an outwardly extending lock release flange 2636 of the spool lock member 2626. The lock release flange 2636 allows a user to slide the spool lock member 2626 axially along the receptacle end 2620R of the spool connector 2620 to selectively engage and disengage the locking projections 2628 relative to the locking cavities 2630 in the spool 2546. Sliding the spool lock member 2626 toward the pyramid adaptor 2604 disengages the locking projections 2628 from the locking cavities 2630 in the spool 2546 and allows the spool to rotate freely. Sliding the spool lock member 2626 away from the pyramid adaptor 2604 moves the locking projections 2628 into the locking cavities 2630 in the spool 2546 and binds the spool 2546 to rotate only with the spool connector 2620. Although not shown, a biasing member may be interposed between the bearing housing 2622 and the spool lock member 2626 to bias the spool lock member 2626 into engagement with the spool 2546.

The spindle formed by the receptacle end 2610R of the fixed shaft 2610 extends through the hollow axial axle 2548 of the actuator 2540 and through the spool connector 2620, and is friction fit into a hollow cap 2638 that is fixed to the lamination adaptor 2631. The cap 2638 extends into the receptacle end 2620R of the spool connector 2620. The cap 2638 axially fixes the fixed shaft 2610 relative to the actuator enclosure 2506.

A hollow cam 2640 circumferentially surrounds the effector end 2610E of the fixed shaft 2610 and a corresponding portion of the actuator 2540. The cam 2640 is axially movable within the actuator enclosure 2506, with travel limits imposed by the bearing housing 2622 and by an inner shoulder 2642 of the actuator enclosure 2506. The cam 2640 further circumferentially surrounds the flange 2606F and the hollow receptacle end 2606R of the movable shaft 2606. Axial movement of the movable shaft 2606 within the actuator enclosure 2506 is limited by engagement of the flange 2606F of the movable shaft 2606 with an inner shoulder 2644 of the cam 2640 and with an outer shoulder 2646 on the effector end 2610E of the fixed shaft 2610. In the illustrated embodiment the cam 2640 is formed by two cam halves 2640A, 2640B. Each of the cam halves 2640A, 2640B has a respective cam slot 2648 that receives a respective one of the actuator arms 2544 of the actuator 2540, with the actuator arms 2544 acting as cam followers. The cam halves 2640A, 2640B also have apertures 2641 for the arms 2611A of the brace 2611. As noted previously, the cam 2640 is axially movable within the actuator enclosure 2506, and this axial movement drives relative movement of the actuator arms 2544 along the cam slots 2648 so that the actuators arms 2544 act as levers to pivot the actuator 2540. Thus, each actuator arm 2544 is a cam follower received in a cam slot 2648 in the cam 2640, whereby linear movement of the cam 2640 drives rotary movement (rocking) of the actuator 2540 (rocker).

A resistive element in the form of a wave spring 2650 is interposed between the movable platform 2602 and the cam 2640. The wave spring 2650 surrounds the effector end 2606E of the movable shaft 2606. The effector end 2606E of the movable shaft 2606 is threaded, and threadedly receives a tension adjustment nut 2652, which is interposed between the movable platform 2602 and the wave spring 2650. Turning the tension adjustment nut 2652 can increase or decrease the preload tension on the wave spring 2650. The wave spring 2650 is trapped between the movable platform 2602 and the actuator 2540. The movable platform 2602 is biased into the distal position by the wave spring 2650, and movement of the movable platform 2602 toward the proximal position (closer to the lamination adaptor 2631) pushes the wave spring 2650 toward the actuator 2540.

Operation of the self-adjusting socket 2500 will now be described.

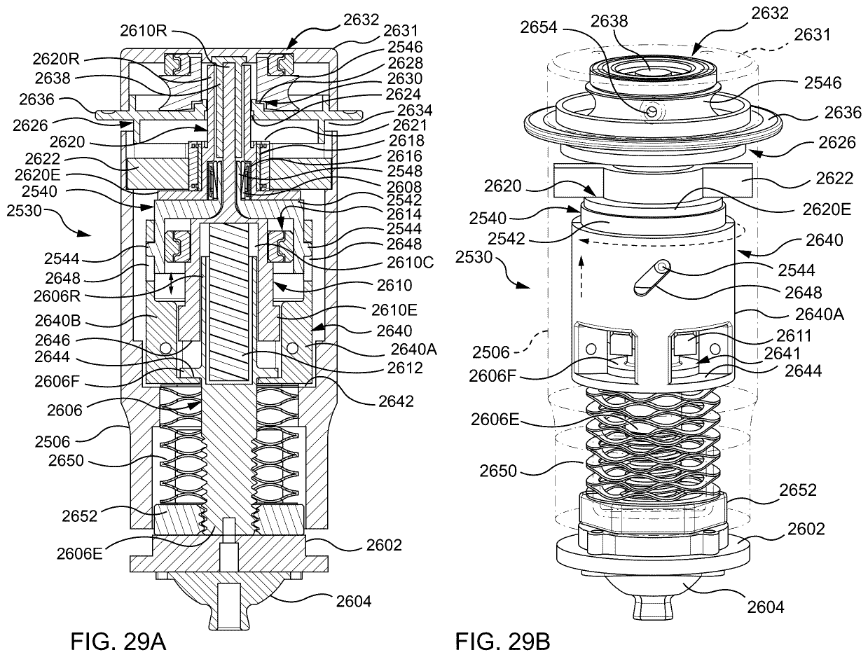

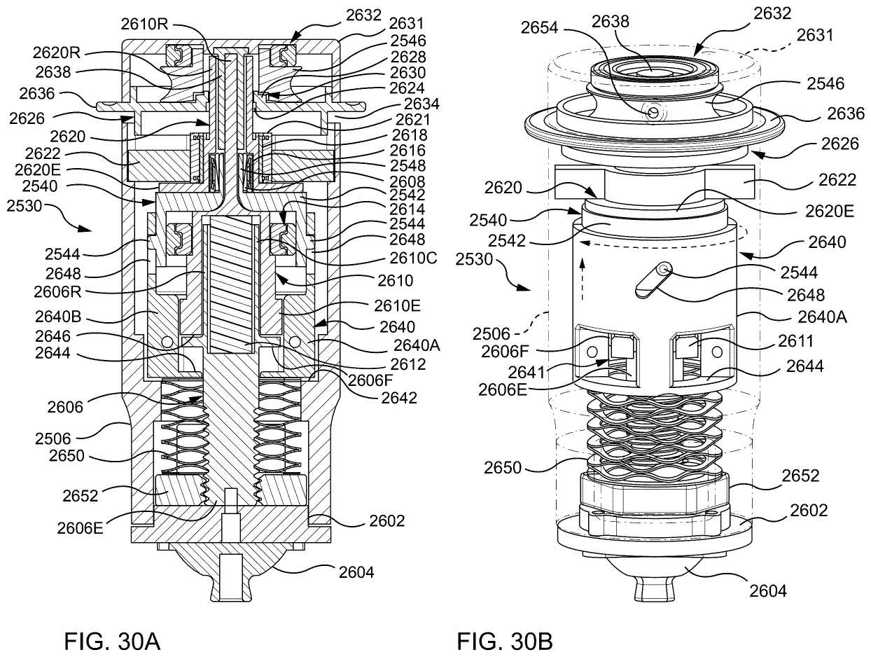

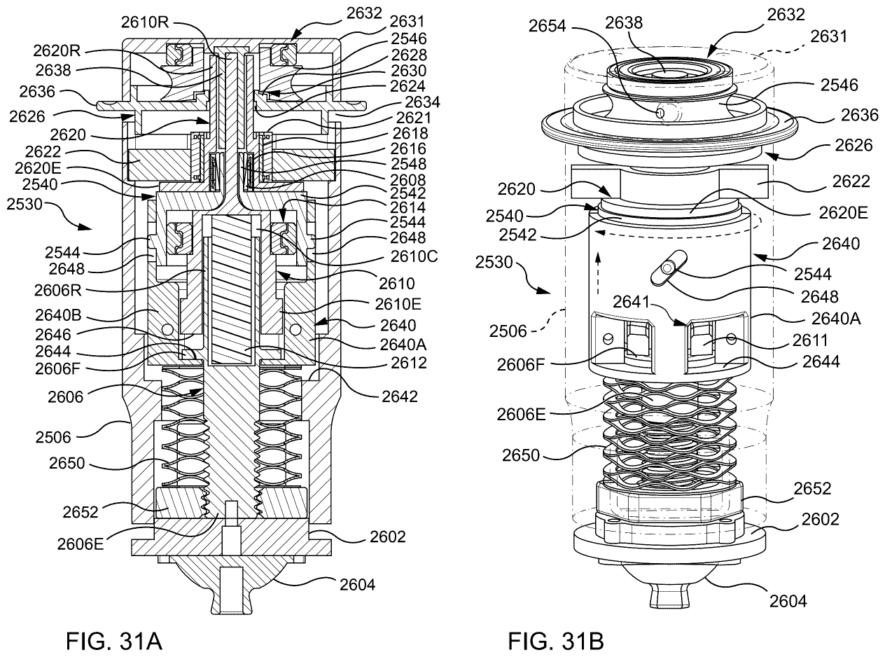

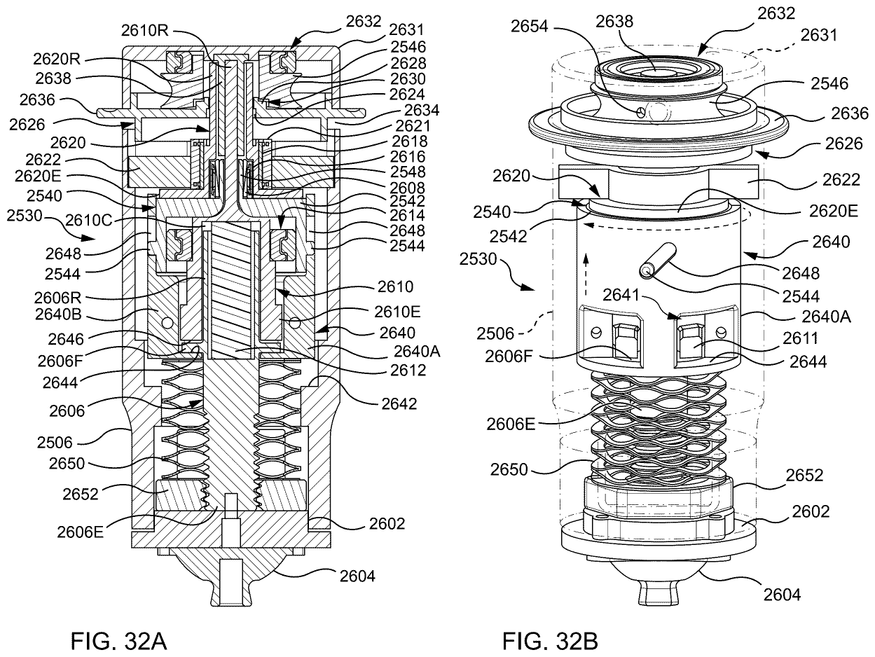

Reference is now made to FIGS. 29A through 32B. In the description that follows, the terms “rest position” and “actuated position” are applied to the actuator arms 2544 since the relative positions of the actuator arms 2544 are clearly visible in the drawings. It will be understood that when the actuator arm 2544 is in the rest position the actuator 2540 will likewise be in the rest position, and conversely when the actuator arm 2544 is in the actuated position the actuator 2540 will likewise be in the actuated position. FIGS. 29A and 29B show the actuator arm 2544 in the rest position with the movable platform 2602 in the distal position, FIGS. 30A and 30B show the actuator arm 2544 in the rest position with the movable platform 2602 in the proximal position, FIGS. 31A and 31B show the actuator arm 2544 moving from the rest position toward the actuated position as the movable platform 2602 moves from the distal position toward the proximal position, and FIGS. 32A and 32B show the actuator arm 2544 in the actuated position with the movable platform 2602 in the proximal position.

FIGS. 29A and 29B represent the configuration of the winch assembly 2530 when a user is not applying weight to the end effector 2515. When the user applies weight to the end effector 2515, the winch assembly 2530 will move either to the configuration in FIGS. 30A and 30B, or through the intermediate configuration in FIGS. 31A and 31B to the configuration in FIGS. 32A and 32B, depending on the tension in the cable 2520. When the user applies weight to the end effector 2515, the force applied by the weight of the user overcomes the resistance of the cushioning element 2612, moving the movable platform 2602 from the distal position (FIGS. 29A and 29B) into the proximal position (FIGS. 30A and 30B and FIGS. 32A and 32B). This movement of the movable platform 2602 applies force to the wave spring 2650 via the tension adjustment nut 2652, since the wave spring 2650 is trapped in the actuator enclosure 2506 between the tension adjustment nut 2652 and the cam 2640, which is movable within the actuator enclosure 2506. As the movable platform 2602 moves from the distal position (FIGS. 29A and 29B) into the proximal position (FIGS. 30A and 30B and FIGS. 32A and 32B), the wave spring 2650 will either act as a rigid body bracing the cam 2640 against the tension adjustment nut 2652 and hence against the movable platform 2602 (FIGS. 31A and 32B), or else the wave spring 2650 will yield against the cam 2640 (FIGS. 30A and 30B). Whether the wave spring 2650 will act as a rigid body or yield depends upon whether the resistance to movement of the cam 2640 is greater than the resistance to further compression of the wave spring 2650. Because the cam 2640 is coupled to the actuator arms 2544 via the cam slots 2648, the resistance to movement of the cam 2640 depends on the resistance to movement of the actuator arms 2544. Moving the actuator arms 2544 rotates the actuator body 2542, which in turn rotates the spool connector 2620, spool lock member 2626 and spool 2546 to further wind the cable 2520 onto the spool 2546 to incrementally increase the tension in the cable 2520. Therefore, the resistance to movement of the cam 2640 depends on the tension in the cable 2520.

More particularly, the actuator arms 2544 projecting through the cam slots 2648 brace the cam 2640 against the actuator arms 2544. Therefore, the cam 2640 cannot move with the movable platform 2602 as the movable platform 2602 moves into the proximal position unless the actuator arms 2544 can move into the actuated position by rotating the actuator body 2542 to pivot the actuator 2540. Since the actuator 2540 is bound to the spool connector 2620 by the one-way needle roller bearing 2616, the actuator 2540 can only pivot if the spool connector 2620 can rotate. Because the spool connector 2620 is rotationally fixed to the spool lock member 2626, the spool connector 2620 can only rotate if the spool lock member 2626 can rotate. When the locking projections 2628 are received in the locking cavities 2630, the spool lock member 2626 can only rotate if the spool 2546 can also rotate. Since the cable 2520 is wound onto the spool 2546 and is under tension, the spool 2546 can only rotate if the existing tension in the cable 2520 can be overcome. Thus, the tension in the cable 2520 propagates back through the spool 2546, spool lock member 2626, spool connector 2620 and one-way needle roller bearing 2616 to resist pivoting of the actuator 2540, thereby resisting movement of the actuator arms 2544 from the rest position to the actuated position and providing resistance to movement of the cam 2640 via the actuator arms 2544 of the cam 2640.

The tension in the cable 2520 tightens the panels 2516 against the residuum, and therefore the amount of tension in the cable 2520 corresponds to the tightness of the retention mechanism comprising the panels 2516. If the tension in the cable 2520 is below a threshold, when the end effector 2515 engages the surface, the winch assembly 2530 moves through the configuration shown in FIGS. 31A and 31B to the configuration shown in FIGS. 32A and 32B, which rotates the spool 2546 to increase tension in the cable 2520. However, when the tension in the cable 2520 is at or above the threshold, when the end effector 2515 engages the surface the winch assembly 2530 moves to the configuration in FIGS. 30A and 30B, which fails to rotate the spool 2546 and therefore will not increase the tension in the cable 2520. The threshold for the tension in the cable 2520 corresponds to the resistance of the wave spring 2650, which may be adjusted by tightening or loosening the tension adjustment nut 2652 so that the threshold is adjustable. Moreover, the resistance of the wave spring 2650 and the cushioning element 2612 need not be identical.

Reference is first made to FIGS. 30A and 30B, which illustrate the scenario where the amount of force required to further compress the wave spring 2650 is less than the resistance to movement of the cam 2640 resulting from the tension in the cable 2520 transmitted back to the actuator arms 2544. More force is required to overcome the tension in the cable 2520 and move the actuator arms 2544 from the rest position to the actuated position than is required to further compress the wave spring 2650. Therefore, the wave spring 2650 yields, so that movement of the movable platform 2602 is not transmitted through the wave spring 2650 to move the cam 2640 and pivot the actuator 2540; the wave spring 2650 acts like a spring rather than a rigid body. The wave spring 2650 is compressed between the tension adjustment nut 2652 and the cam 2640 as the movable platform 2602 and the movable shaft 2606 moves, with the cam 2640 remaining axially fixed inside of the actuator enclosure 2506, effectively pinned by the actuator arms 2544. The resistance of the wave spring 2650 cannot overcome the tension in the cable 2520, so the wave spring 2650 yields (in this case, is compressed). Accordingly, instead of the actuator arms 2544 moving toward the actuated position, the actuator arms 2544 are maintained in position by the tension in the cable 2520 and the wave spring 2650 is compressed between the cam 2640 and the tension adjustment nut 2652. Thus, when the tightness of the retention mechanism comprising the panels 2516 has reached the threshold, on each further step the wave spring 2650 yields to absorb the motion of the movable platform 2602, rather than transmitting the motion of the movable platform 2602 to the actuator 2540. When the wave spring 2650 yields instead of transmitting motion, the actuator 2540 will fail to cycle on each further step, thereby inhibiting further tightening of the retention mechanism beyond the threshold.

Reference is now made to FIGS. 31A and 31B, which illustrate the scenario where the amount of force required to further compress the wave spring 2650 exceeds the resistance to movement of the cam 2640 resulting from the tension in the cable 2520 (i.e. tension in the cable 2520 is below the threshold). In this scenario, the wave spring 2650 does not yield, but acts as a rigid body that transmits the motion of the movable platform 2602 to the cam 2640 so that the cam 2640 moves axially within the actuator enclosure 2506 toward the lamination adaptor 2631. Accordingly, movement of the movable platform 2602 into the proximal position will drive the cam 2640 linearly, forcing the actuator arms 2544 to move along the cam slots 2648, which rotates the actuator body 2542. Rotation of the actuator body 2542 rotates the spool connector 2620, which rotates the spool lock member 2626, which (when the locking projections 2628 are received in the locking cavities 2630) rotates the spool 2546 to further wind the cable 2520 onto the spool 2546 to incrementally increase the tension in the cable 2520. Thus, where the tension in the cable 2520 is below the threshold, where there is movement of the movable platform 2602 toward the proximal position, the wave spring 2650 transmits this movement via the cam 2640 to the actuator arm 2544 to pivot the actuator 2540 and thereby index the spool 2546. Accordingly, when the tightness of the retention mechanism comprising the panels 2516 and cable 2520 is below a desired threshold, each step transmits motion across the wave spring 2650 to the actuator 2540 to cycle the actuator 2540. Each movement of the actuator arms 2544 into the actuated position rotates the actuator body 2542, which (via the one-way needle roller bearing 2616) rotates the spool connector 2620 and thereby (via the spool lock member 2626) rotates the spool 2546 to wind the cable 2520 onto the spool 2546 to incrementally increase the tension in the cable 2520, which further tightens the retention mechanism.

More particularly, movement of the actuator arm 2544 from a rest position (FIGS. 29A and 29B) through an intermediate position (FIGS. 31A and 31B) to an actuated position (FIGS. 32A and 32B) will rotate (“rock”) the actuator body 2542 (rotation to the left in the Figures). The one-way needle roller bearing 2616 rotationally binds the spool connector 2620 to the actuator body so that rotation of the actuator body 2542 drives rotation of the spool connector 2620 (rotation to the left in the Figures). The one-way clutch bearing 2621 on the spool connector 2620 permits this rotation, the spool connector 2620 can rotate leftward within the one-way clutch bearing 2621 until the actuator arm 2544 reaches the actuated position (see FIGS. 32A and 32B). Movement of the actuator arm 2544 from the rest position (FIGS. 29A and 29B) through the intermediate position (FIGS. 31A and 31B) to the actuated position (FIGS. 32A and 32B) will incrementally rotate the spool connector 2620, which incrementally rotates the spool lock member 2626, which incrementally rotates the spool 2546 coupled thereto in the winding direction of the spool 2546. This incrementally winds the cable 2520 (see FIG. 25) further onto the spool 2546 and incrementally increases the tension in the cable 2520. The one-way clutch bearing 2621 then resists rotation in the unwinding direction of the spool 2546 so that tension on the cable 2520 is maintained.

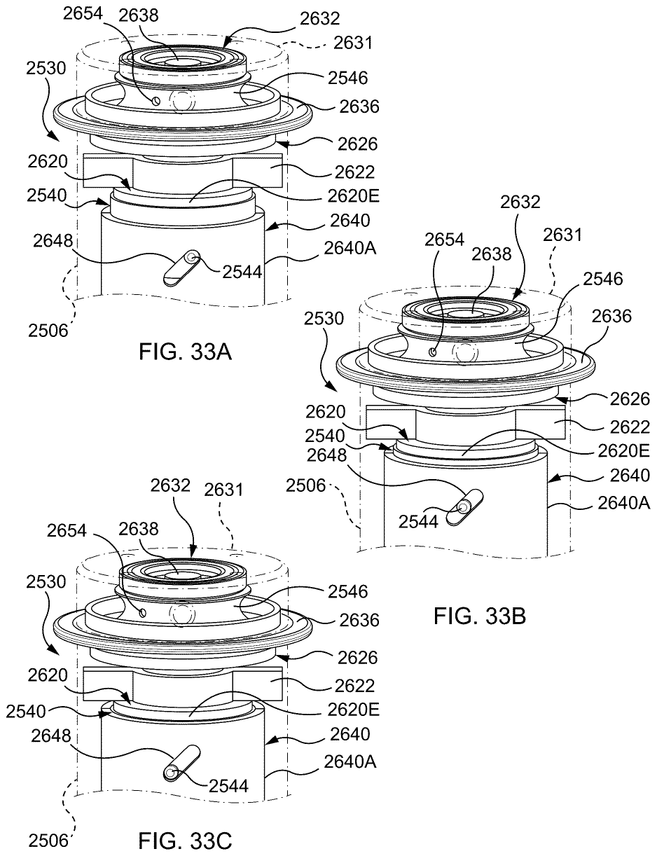

From the actuated position shown in FIGS. 32A and 32B, the actuator arm 2544 can reciprocate back to the rest position shown in FIGS. 29A and 29B, thereby rotating the actuator body 2542 back in the opposite rotational direction (rotation to the right in the Figures). The one-way needle roller bearing 2616 permits the axle 2548 of the actuator 2540 to slip within the spool connector 2620 so that the actuator 2540 can rotate rightward until the actuator arm 2544 returns to the rest position shown in FIGS. 29A and 29B. Simultaneously, the one-way clutch bearing 2621 inhibits the spool connector 2620 from rotating back about the one-way needle roller bearing 2616 on the axle 2548 of the actuator 2540. Thus, each cycle of the actuator 2540 from the rest position (FIGS. 29A and 29B) through the intermediate position (FIGS. 31A and 31B) to the actuated position (FIGS. 32A and 32B) incrementally rotates (indexes) the spool connector 2620. Since rotation of the spool connector 2620 will result in rotation of the spool 2546 via the spool lock member 2626, each cycle of the actuator 2540 from the rest position (FIGS. 29A and 29B) to the actuated position (FIGS. 32A and 32B) and back to the rest position (FIGS. 29A and 29B) incrementally rotates (indexes) the spool 2546. Thus, each cycle of the actuator 2540 indexes the spool 2546 to wind the cable 2520 onto the spool 2546 to incrementally increase the tension in the cable 2520. Accordingly, the actuator 2540 is configured to reciprocally cycle between a rest position and an actuated position and to act through the mechanical linkage comprising the cables 2520, via the winch assembly 2530, to incrementally tighten the retention mechanism comprising the panels 2516 to incrementally tighten the retention mechanism against the residuum on each movement of the actuator 2540 into the actuated position and leave the retention mechanism further incrementally tightened upon each return of the actuator 2540 to the rest position. FIGS. 33A through 33C more clearly show how movement of the spool 2546 corresponds to the position of the actuator arms 2544 in the cam slots 2648 as the cam 2640 moves axially; movement of the spool 2546 is shown by movement of a cable connection aperture 2654 on the spool 2546.

When the user removes the weight from the end effector 2515, the cushioning element 2612 returns the movable platform 2602 from the proximal position (FIGS. 30A and 30B and FIGS. 32A and 32B) to the distal position (FIGS. 29A and 29B).

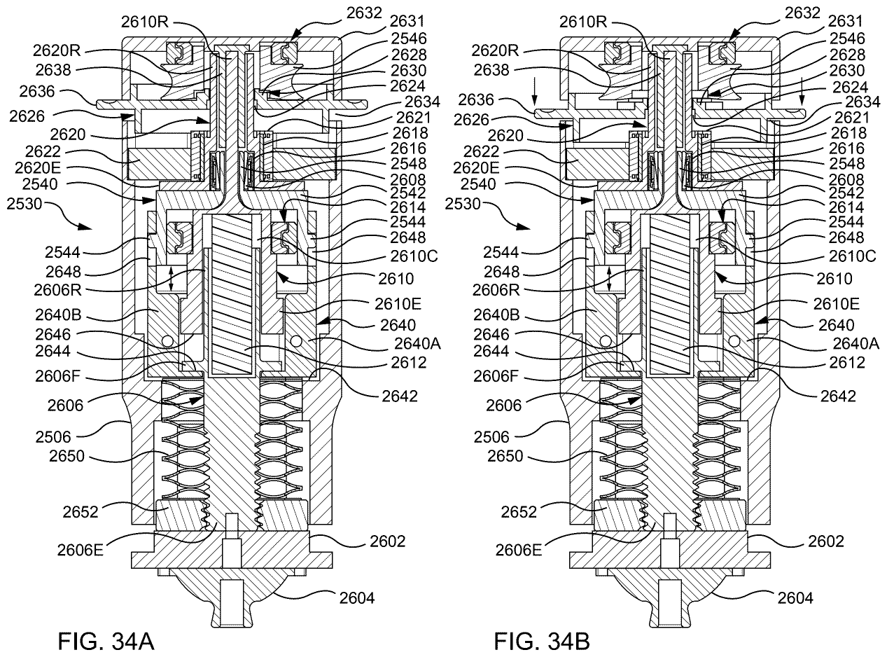

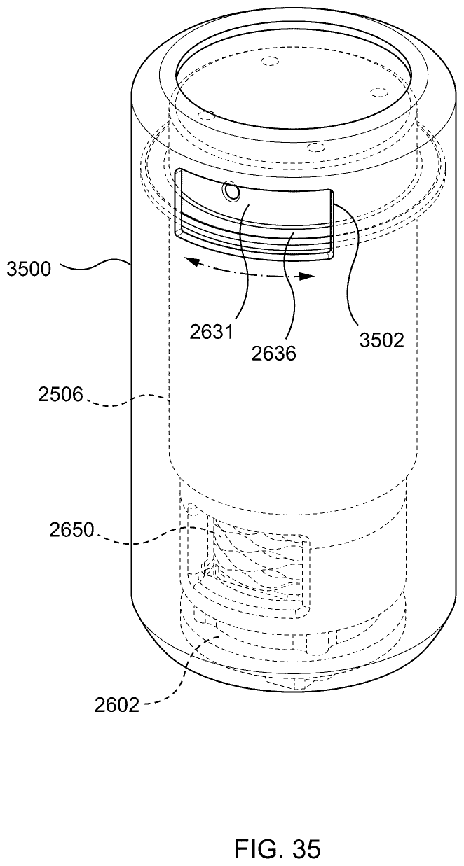

The actuator enclosure 2506 carries a releasable locking mechanism. The releasable locking mechanism is provided by the movable spool lock member 2626. As best seen in FIGS. 34A and 34B, a user can manipulate the lock release flange 2636 of the spool lock member 2626 to selectively engage and disengage the locking projections 2628 relative to the locking cavities 2630 in the spool 2546. Sliding the spool lock member 2626 toward the pyramid adaptor 2604 disengages the locking projections 2628 from the locking cavities 2630 in the spool 2546 and allows the spool 2546 to rotate freely, thereby releasing the locking mechanism. Sliding the spool lock member 2626 away from the pyramid adaptor 2604 moves the locking projections 2628 into the locking cavities 2630 in the spool 2546 and binds the spool 2546 to rotate only with the spool connector 2620, maintaining tightness of the retention mechanism against the residuum after each cycle of the actuator 2540.

In addition, when the spool lock member 2626 is engaged with the spool 2546, the spool lock member 2626 can provide a manual tightening mechanism for tightening the retention mechanism. FIG. 35 shows how an outer shroud 3500 around the actuator enclosure 2506 may be provided with a window 3502 through which a user can access the lock release flange 2636 of the spool lock member 2626 to selectively engage and disengage the locking projections 2628 and also rotate the lock release flange 2636 in the winding direction (leftward in this case) to rotate the spool 2546 to manually tighten the cable 2520 (FIG. 24).

Accordingly, as has been shown with respect to FIGS. 29A through 32B, reciprocal movement of the movable platform 2602 into the proximal position and back to the distal position cycles the actuator 2540 only where the resistance to compression of the wave spring 2650 exceeds the resistance to movement of the actuator 2540 resulting from the tension in the cable 2520 so that the wave spring 2650 transmits the movement of the movable platform 2602 to the actuator 2540 instead of yielding to the movement of the movable platform 2602.

Thus, in the illustrated embodiment, the wave spring 2650, actuator 2540, one-way needle roller bearing 2616, and spool connector 2620 provide a mechanical interface to transmit movement of the end effector 2515 of the lower limb prosthesis toward the residuum resulting from a step to a tensioner comprising the spool lock member 2626, and spool 2546, with resistance of the mechanical interface provided by the wave spring 2650 (e.g. a spring).

The winch assembly 2530 shown in FIGS. 25 through 33C may be provided without any receptacle body 2504 or end effector 2515, for example as a kit of parts, with or without the lamination adaptor 2631, for use by a trained prosthetist in constructing a socket 2500.

As noted above, the panels 116, 1616, 2516 shown for the illustrative sockets 100, 1600, 2500 are configured for a transtibial amputee. The same mechanism, including the winch assembly 130, 1630, 2330, 2530, movable platform 146, 1646, 2346, 2602 and resistive elements 1050, 1750, 2450, 2650 may also be used to apply tension to cables for tightening panels configured for a transfemoral amputee and thus the present disclosure encompasses a socket for a lower limb prosthesis for a transfemoral amputee.

The apparatus described above provide some illustrative, non-limiting implementations of a method for securing a residuum in a socket of a lower limb prosthesis. Motion from steps taken with the lower limb prosthesis is transmitted across a resilient resistive element to an actuator to cycle the actuator, with each cycle of the actuator incrementally tightening a retention mechanism against the residuum, until a tightness threshold of the retention mechanism is reached. After the tightness threshold is reached, motion from further steps taken with the lower limb prosthesis is transmitted into the resistive element and the resistive element yields and absorbs the motion so that the actuator fails to cycle on each further step, inhibiting further tightening of the retention mechanism beyond the threshold.

In the illustrative implementations described above, each cycle of the actuator 140, 1640, 2340 incrementally winds a cable 120, 1620, 2320, 2520 around a spool 1006, 1706, 2406, 2546 to increase tension in the cable 120, 1620, 2320, 2520 which is coupled to the retention mechanism comprising the panels 116, 1616, 2516 such that increasing the tension in the cable 120, 1620, 2320, 2520 tightens the retention mechanism by forcing the panels 116, 1616, 2516 inwardly against the residuum, with a spring serving as the resistive element 1050, 1750, 2450, 2650. However, this is merely an illustrative, non-limiting mechanical implementation of the method, and other mechanical implementations are also contemplated.

Certain non-limiting illustrative embodiments have been described by way of example. It will be apparent to persons skilled in the art that a number of variations and modifications can be made without departing from the scope of the claims.

The following listing of reference characters is provided for convenience of reference only, and no limitation is implied:

### First Illustrative Embodiment

100 Self-adjusting socket (generally)
102 Housing of self-adjusting socket
104 Receptacle body of housing
106 Actuator enclosure (winch body) of housing
108 Open end of receptacle body
110 Support end of receptacle body
112 Residuum receptacle
114 Mounting block
116 Panels
118 Openings in receptacle body
120 Cables
122 Cable tunnels in panels
124 Cable guides on receptacle body
126 Port
128 Passageway through support end of receptacle body
130 Winch assembly
132 Locking post
134 Locking lugs
136 Locking aperture
138 Locking recesses
140 Actuators
146 Movable platform
148 Dovetail guide follower
150 Guide channel
152 Elongate inward projection
154 Interior sidewall
1002 Actuator body
1004 Actuator arm
1006 Spool
1008 Winch cavities
1010 Bushing aperture
1012 Bearing aperture
1014 Winch needle bearing
1014R Winch needle bearing rollers
1016 Winch bushing
1018 Actuator needle bearing
1018R Actuator needle bearing rollers
1020 Main winch shaft
1022 Locking ball bearings
1024 Release shaft
1026 Release spring
1028 Eye of release shaft
1030 Eye of spring retainer
1032 Spring retainer
1034 End cap of spring retainer
1036 Locating apertures
1038 Annular wall of main winch shaft
1040 Locking channels
1042 Inner surface of spool
1044 Manual tensioning knob
1046 Cushioning springs
1048 Locating studs
1050 Resistive elements
1052 Cylinder barrel
1054 Setscrew
1056 Piston
1058 Head of cylinder
1060 Piston rod
1062 Rod aperture
1063 T-shaped slot in actuator arm
1064 Piston rod connector
1066 Release handle
1068 Annular recess in release shaft
1070 Beveled edge of annular recess
1072 Main body of release shaft

### Second Illustrative Embodiment

100 Self-adjusting socket (generally)
102 Housing of self-adjusting socket
1604 Receptacle body of housing
1606 Actuator enclosure (winch body) of housing
1608 Open end of receptacle body
16
10 Support end of receptacle body
1612 Residuum receptacle
1615 End effector
16
16 Panels
1618 Openings in receptacle body
1620 Cables
1622 Cable guides in panels
1624 Cable guides on receptacle body
1630 Winch assembly
1640 Actuator
1646 Movable platform
1648 Platform flange
1650 Platform guide channels
1652 Platform guide rods
1654 Platform bushings
1656 Guide rod holes
1674 Lamination plate
1676 Adjustment mechanism retaining ring
1678 Enclosure flange
1680 Distal surface of lamination plate
1682 Proximal surface of adjustment mechanism retaining ring
1684 Locating pin apertures
1686 Locating pins
1688 Locating apertures
1690 Friction sleeve
1692 Platform base
1693 Outwardly necked distal end of actuator enclosure
1694 Platform retainer ring
1696 Inner shoulder on actuator enclosure
1698 Platform retainer flange on platform retainer ring
1702 Actuator body
1704 Actuator arm
1706 Spool
1708 Winch cavity
1710 First bearing aperture
1712 Second bearing aperture
1714 Winch needle bearing
1714R Winch needle bearing rollers
1716 Winch roller bearing
1718 Actuator needle bearing
1718R Actuator needle bearing rollers
1720 Main winch shaft
1722 Crossbar arms of T-shaped end of release shaft
1724 Release shaft
1726 Release spring
1728 T-shaped end
1730 Retainer aperture in actuator enclosure
1732 Spring retainer
1734 Central post of spring retainer
1736 Slots through spool end of main winch shaft
1738 Spool end of main winch shaft
1740 Locking channels
1743 Knob end of main winch shaft
1744 Manual tensioning knob
1745 Cavity in manual tensioning knob
1746 Cushioning element
1748 Cushioning element guide tube
1750 Resistive element
1752 Cylinder barrel
1754 Cylinder setscrew
1755 Guide tube setscrew
1756 Piston
1758 Head of cylinder
1760 Piston arm
1762 Articulation aperture
1764 Piston connector pin
1765 Actuator connector pin
1766 Release button
2202 Spool barrel
2204 Spool flange
2206 Cable-retaining flange
2208 Cable grooves
2210 Cable anchor wells
2214 Arbor hole
2216 Surface of arbor hole
2218 Crenate ring
2220 Gladiate elements
2222 Tapered ends of gladiate elements

### Third Illustrative Embodiment

2306 Actuator enclosure
2320 Cable
23
26 Port
2328 Passageway
2329 Idler pulley
2330 Winch assembly
2340 Actuator (pinion)
2346 Movable platform
2378 Enclosure flange
2390 Friction sleeve
2392 Platform base
2394 Proximal surface of cushioning interface wall
2402 Pinion body
2404 Pinion teeth
2406 Spool
2408 Winch cavity
2418 Pinion needle bearing
2420 Main winch shaft
2424 Release shaft
2446 Cushioning ring
2448 Cushioning interface wall
2450 Resistive element
2452 Cylinder barrel
2456 Piston
2458 Head of cylinder barrel
2460 Rack
2462 Piston slots
2464 Rack teeth

### Fifth Illustrative Embodiment

2500 Socket
2502 Housing
2504 Receptacle body
2506 Actuator enclosure
2508 Open end of receptacle body
2510 Support end of receptacle body
2512 Residuum receptacle
2515 End effector
2516 Panels
2518 Openings in receptacle body
2520 Cable
2522 Tubular cable guides
2524 Covered cable guides
2530 Winch assembly
2540 Actuator
2542 Actuator body
2544 Actuator arms
2546 Spool
2548 Hollow axial axle of actuator
2602 Movable platform
2604 Pyramid adaptor
2606 Movable shaft
2606E Effector end of movable shaft
2606F Flange
2606R Receptacle end
2608 Sleeve bearing
2610 Fixed shaft
2610C Cylindrical axial cavity in effector end of fixed shaft
2610E Effector end of fixed shaft
2610R Receptacle end of fixed shaft
2611 Brace for fixed shaft
2611A Arms of brace
2612 Cushioning element
2613 Brace slots
2614 Two-way roller bearing between actuator and fixed shaft
2616 One-way needle roller bearing (inner one-way bearing)
2618 Bushing
2620 Spool connector
2620E Flanged effector end of spool connector
2620R Receptacle end of spool connector
2621 One-way clutch bearing (outer one-way bearing)
2622 Bearing housing
2624 Central aperture of spool lock member
2626 Spool lock member
2628 Locking projections on spool lock member
2630 Locking cavities in spool
2631 Lamination adaptor
2632 Two-way roller bearing
2634 Opening between lamination adaptor and actuator enclosure
2636 Lock release flange of spool lock member
2638 Hollow cap
2640 Hollow cam
2640A Hollow cam half
2640B Hollow cam half
2641 Apertures in cam halves for the arms of brace
2642 Inner shoulder of actuator enclosure
2644 Inner shoulder of cam
2646 Outer shoulder on effector end of the fixed shaft
2648 Cam slots
2650 Wave spring
2652 Tension adjustment nut
2654 Cable connection aperture
3500 Outer shroud around actuator enclosure
3502 Window in outer shroud

What is claimed is:

1. A socket adjustment mechanism for a lower limb prosthesis, comprising:
an actuator enclosure connectable to a receptacle body having a residuum receptacle;
at least one actuator disposed within the actuator enclosure and connectable to a residuum retention mechanism associated with the residuum through a respective mechanical linkage, the at least one actuator configured to reciprocally cycle between a rest position and an actuated position and to act through the respective mechanical linkage to incrementally tighten the retention mechanism against the residuum on each movement of the at least one actuator into the actuated position and leave the retention mechanism further incrementally tightened upon each return of the actuator to the rest position;
a releasable locking mechanism carried by the actuator enclosure and configured to maintain tightness of the retention mechanism against the residuum after each cycle of the at least one actuator;
the actuator enclosure configured so that each step transmits motion to a respective resilient resistive element coupled to a respective one of the at least one actuator, wherein:
when a tightness of the retention mechanism is below a threshold, each step transmits motion across the respective resistive element to the respective actuator to cycle the respective actuator to further tighten the retention mechanism; and
when the tightness of the retention mechanism has reached the threshold, on each further step the respective resistive element yields to absorb the motion, so that the respective actuator fails to cycle on each further step, inhibiting further tightening of the retention mechanism beyond the threshold.

2. The socket adjustment mechanism of claim 1, wherein the at least one actuator is a single actuator.

3. The socket adjustment mechanism of claim 1, further comprising a manual tightening mechanism for tightening the retention mechanism.

4. The socket adjustment mechanism of claim 1, further comprising:
a housing comprising the residuum receptacle; and
the retention mechanism;
wherein the retention mechanism is carried by the housing and is configured for retaining a residuum within the residuum receptacle;
wherein:
the actuator enclosure is coupled to the residuum receptacle via the housing; and
the at least one actuator is coupled to the retention mechanism through the respective mechanical linkage.

5. The socket adjustment mechanism of claim 4, wherein:
the retention mechanism comprises at least one panel movably carried by the housing;
the at least one panel being movable inwardly and outwardly relative to the residuum receptacle;
the at least one actuator is configured to act through the respective mechanical linkage to incrementally move the at least one panel inwardly to tighten the at least one panel against the residuum on each cycle of the at least one actuator.

6. The socket adjustment mechanism of claim 5, wherein the at least one panel comprises a plurality of panels arranged circumferentially about the residuum receptacle.

7. The socket adjustment mechanism of claim 6, where the panels are disposed in respective openings so as to be inwardly and outwardly displaceable relative to the housing.

8. The socket adjustment mechanism of claim 7, wherein:
the mechanical linkage comprises at least one cable coupled to at least one of the panels;
each respective actuator is configured to incrementally increase tension in a respective one of the at least one cable on each cycle of the respective actuator; and
incrementally increasing the tension on the respective cable moves the respective panels inwardly relative to the residuum receptacle.

9. The socket adjustment mechanism of claim 8, wherein:
the actuator enclosure carries a movable platform;
the platform is reciprocally movable relative to the actuator enclosure between a distal position and a proximal position;
the platform is biased into the distal position;
the at least one actuator is carried by the actuator enclosure between the residuum receptacle and the platform;
the respective resistive element is trapped between the platform and the respective actuator whereby movement of the platform toward the proximal position pushes the resistive element toward the respective actuator;
wherein reciprocal movement of the platform into the proximal position and back to the distal position cycles the respective actuator only where a resistance to compression of the respective resistive element exceeds a resistance to movement from the tension in the respective cable so that the respective resistive element transmits the movement of the platform to the respective actuator instead of yielding to the movement of the platform.

10. The socket adjustment mechanism of claim 9, wherein:
each actuator comprises a rocker coupled to a respective spool;
each cycle of the rocker indexes the spool to wind the respective cable onto the spool to incrementally increase the tension in the respective cable.

11. The socket adjustment mechanism of claim 10, wherein:
each rocker comprises a respective outwardly extending actuator arm that acts as a lever to pivot the rocker; and
where the resistance to compression of the respective resistive element exceeds a resistance to movement from the tension in the cable, the resistive element transmits the movement of the platform into the proximal position to the actuator arm to pivot the rocker and thereby index the spool.

12. The socket adjustment mechanism of claim 11, wherein:
a linearly movable cam is interposed between the resistive element and the rocker;
each actuator arm is a cam follower received in a cam slot in the cam, whereby linear movement of the cam drives rotary movement of the rocker.

13. The socket adjustment mechanism of claim 11, wherein the at least one resistive element is at least one spring.

14. The socket adjustment mechanism of claim 1, wherein the at least one resistive element is at least one spring.

15. A method for securing a residuum in a socket of a lower limb prosthesis, the method comprising:
transmitting motion from steps taken with the lower limb prosthesis across a resilient resistive element to an actuator to cycle the actuator between a rest position and an actuated position, wherein each cycle of the actuator incrementally tightens a retention mechanism against the residuum, until a tightness threshold of the retention mechanism is reached; and
after the tightness threshold is reached, transmitting motion from further steps taken with the lower limb prosthesis into the resistive element wherein the resistive element yields and absorbs the motion so that the actuator fails to cycle on each further step, inhibiting further tightening of the retention mechanism beyond the threshold.

16. The method of claim 15, wherein:
each cycle of the actuator incrementally winds a cable around a spool to increase tension in the cable;
the cable is coupled to the retention mechanism and increasing the tension in the cable tightens the retention mechanism.

17. The method of claim 16, wherein increasing the tension in the cable tightens the retention mechanism by forcing a panel inwardly against the residuum.

18. The method of claim 15, wherein the resistive element is a spring.

19. A method for tightening a panel in a receptacle for a residuum, the method comprising:
applying incremental tension across the panel to move the panel inwardly relative to the receptacle, wherein:
the incremental tension is applied by transmission of movement of an end effector of a lower limb prosthesis toward the residuum through a mechanical interface to a tensioner; and
the movement is transmitted to the tensioner only when a resistance of the mechanical interface exceeds a current tension applied by the tensioner.

20. The method of claim 19, wherein the resistance of the mechanical interface is provided by at least one spring.

21. The method of claim 19, wherein the tensioner comprises a winch.
